(19) United States
(12) Patent Application Publication
(10) Pub. No.: US 2021/0332366 A1
(43) Pub. Date: Oct. 28, 2021

(54) Novel Small Activating RNA

(71) Applicant: Ractigen Therapeutics, Nantong City (CN)

(72) Inventors: Longcheng LI, Nantong City (CN); Moorim KANG, Nantong City (CN); Robert F. PLACE, Nantong City (CN); Jiancheng WU, Nantong City (CN)

(21) Appl. No.: 16/755,299

(22) PCT Filed: Apr. 10, 2019
(86) PCT No.: PCT/CN2019/082149

§ 371 Date: Apr. 10, 2020

(30) Foreign Application Priority Data
Apr. 10, 2018 (CN) 201810317366.X

(51) Int. Cl. C12N 15/113; A61P 35/00

(52) U.S. Cl. CPC C12N 15/1135; C12N 2320/34; C12N 2310/34; A61P 35/00

(57) ABSTRACT

saRNAs are provided in the present invention. The saRNAs are composed of a first oligonucleotide strand containing 17 to 30 nucleotides and a second oligonucleotide strand containing 17 to 30 nucleotides. Sequences of at least 15 nucleotides in length are complementary in the two oligonucleotide strands, and the unpaired terminal nucleotides form overhangs. The first oligonucleotide strand or the second oligonucleotide strand has more than 75% homology or complementarity with any continuous fragment of 16 to 35 nucleotides in length in the promoter of the target gene. The second oligonucleotide strand has an overhang composed of 1 to 4 nucleotides at 3′ end. saRNAs of the present invention can upregulate target gene expression more effectively while reducing off-target effects.

### CROSS-REFERENCE TO RELATED APPLICATION

This application is a Section 371 of International Application No. CT/CN2019/082149, published in the Chinese language on Oct. 17, 2019, under International Publication No. WO 2019/196887 A1, which claims priority to Chinese Application No. 201810317366.X, filed on Apr. 10, 2018, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “065786_1US1_Sequence_Listing_Sustitute” and a creation date of Oct. 28, 2020 and having a size of 115 KB. The sequence listing submitted via EFS-Web is part of the specification and is herein incorporated by reference in its entirety.

### FIELD OF THE INVENTION

The present invention relates to the field of molecular biology, and in particular to up-regulating gene expression with a double-stranded small RNA targeting a gene promoter.

### BACKGROUND OF THE INVENTION

Double-stranded small nucleic acid molecules including chemically synthesized oligoribonucleotides (such as small activating RNA (saRNA)) and naturally occurring oligoribonucleotides (such as micro ribonucleic acid (miRNA)) have been proved to be capable of targeting regulatory sequences (such as promoter sequences) of protein-coding genes in a sequence-specific manner to up-regulate gene expression at the transcriptional and epigenetic level, a phenomenon known as RNA activation (RNAa) (Li, Okino et al. (2006) Proc Natl Acad Sci USA 103:17337-17342; Janowski, Younger et al. (2007) Nat Chem Biol 3:166-173; Place, Li et al. (2008) Proc Natl Acad Sci USA 105:1608-1613; Huang, Place et al. (2012) Nucleic Acids Res 40:1695-1707; Li (2017) Adv Exp Med Biol 983:1-20). Studies have shown that RNAa is an endogenous molecular mechanism evolutionarily conserved from Caenorhabditis elegans to the human being (Huang, Qin et al. (2010) PLoS One 5:e8848; Seth, Shirayama et al. (2013) Dev Cell 27:656-663; Turner, Jiao et al. (2014) Cell Cycle 13:772-781).

Safe strategies to selectively enhance gene and/or protein production remain a challenge in gene therapy. Viral-based systems have inherent drawbacks including adverse effects on host genome integrity and immunological consequences. RNAa, with the advantages of being able to activate endogenous genes without the risk of altering the genome, represents a new strategy to stimulate target gene expression.

Cyclin-dependent kinase (CDK) inhibitor p21WAF1/CIP1 (p21) is a mediator of several anti-growth pathways and considered a potent tumor suppressor gene in cancer cells (Harper, Adami et al. (1993) Cell 75:805-816). In fact, overexpression of p21 by ectopic vectors or stimulation of endogenous transcription inhibits tumor growth both in vitro and in vivo (Harper, Adami et al. (1993) Cell 75:805-816; Eastham, Hall et al. (1995) Cancer Res 55:5151-5155; Wu, Bellas et al. (1998) J Exp Med 187:1671-1679; Harrington, Spitzweg et al. (2001) J Urol 166:1220-1233). As such, selective activation of p21 can possess therapeutic application for regulating cell growth and treatment of disease (e.g. cancer).

### SUMMARY OF THE INVENTION

The present invention provides a type of oligonucleotide molecules and a method that can upregulate gene expression in a cell in a targeted manner. By introducing into cells, the oligonucleotide molecule targeting the promoter sequence of a gene, the method can effectively upregulate the expression of the targeted gene and produce an effective biological effect. The oligonucleotides of the present invention are structurally-optimized double-stranded ribonucleic acid molecules, which is also known as small activating RNA (saRNA).

The present invention provides a saRNA, comprising a first oligonucleotide strand of 17 to 30 nucleotides in length and a second oligonucleotide strand of 17 to 30 nucleotides in length, and a region of at least 15 nucleotides in length in the first oligonucleotide strand that is complementary to the second oligonucleotide strand. The first oligonucleotide strand or the second oligonucleotide strand has more than 75% (such as more than 80%, more than 85%, more than 90%, more than 95%, more than 99%, or 100%) sequence homology or complementarity with any continuous fragment of 15 to 30 nucleotides in length in the promoter of a target gene. One end of a duplex formed by the first oligonucleotide strand and the second oligonucleotide strand is a blunt end, i.e. having no overhang structure, and the other end of the duplex can have an overhang consisting of 1 to 4 nucleotides (such as 1, 2, 3 or 4 nucleotides) formed at the terminus of the first oligonucleotide strand or the second oligonucleotide strand. In a specific embodiment, one end of the duplex formed by the first oligonucleotide strand and the second oligonucleotide strand in the aforementioned saRNA is a blunt end, and the other end can have an overhang of 2 or 3 nucleotides formed at the terminus of the first oligonucleotide strand or the second oligonucleotide strand.

In the aforementioned saRNA, nucleotides of the overhang can be selected from T (thymine), U (uracil), or a “natural” nucleotide, for example, the overhang is selected from dTdTdT, dTdT, UUU, UU, or 2 or 3 continuous natural nucleotides (such as A, T, G and C). The natural nucleotide overhang in the present invention means that nucleotides overhanging at the terminus of the first oligonucleotide strand or the second oligonucleotide strand are homologous to or complementary with nucleotides at the corresponding position in a target sequence.

In the aforementioned saRNA, the 5′ terminus of the first oligonucleotide strand or the second oligonucleotide strand is at the blunt end of the duplex, and there are 1 to 3 nucleotides of the first to the third nucleotide from the 5′ terminus in the first or second oligonucleotide strand mispaired with nucleotides at the corresponding position in the other strand. Preferably, the mispairing involves mispaired cytosines. There are at least two patterns for the cytosine mispairing described herein. In one pattern, the nucleotides on one strand are cytosine nucleotides (C), while the guanine nucleotides (G) to be paired with the cytosine nucleotides by Watson-Crick base-pairing are replaced by non-guanine nucleotides (such as A, T or U). In the other pattern, the nucleotides on one strand are non-guanine nucleotides, such as A, U, or T, and the original nucleotides on the other strand to be paired with A, U, or T by Watson-Crick base-pairing are replaced with cytosine nucleotides (C).

In the saRNA provided in the invention, the length of the duplex formed by the first oligonucleotide strand and the second oligonucleotide strand, excluding the overhang, is 17 to 24 nucleotides, such as 17, 18, 19, 20, 21, 22, 23, or 24 nucleotides; and preferably, the length of the duplex formed by the first oligonucleotide strand and the second oligonucleotide strand, excluding the overhang, is 18 to 20 nucleotides.

The present invention further provides the use of the aforementioned saRNA in the preparation of a formulation, such as a drug, for activating or upregulating the expression of a target gene in a cell. The saRNA can be introduced into the cell directly. The cell can be a mammal cell, and preferably a human cell. The human cell can be present in a human body, or can be cultured in vitro, such as an isolated cell line, a cell strain, or a primary cell.

The human body can suffer from a disease caused by the defect and/or deficiency of expression of a target gene. Administration of an effective amount of an saRNA of the invention can, for example, treat the disease.

The target gene for the saRNA, in one case, is human p21. In other cases, the target gene can be other pathogenic genes resulting in diseases due to a gene expression defect and/or deficiency, such as KLF4, NKX3-1, and VEGFA.

If the target gene is p21 or one of the other genes, the saRNA can activate or upregulate the expression of the target gene by at least 10%, such as more than 10%, more than 20%, more than 30%, more than 40%, more than 50%, more than 60%, more than 70%, more than 80%, more than 90%, or more than 100%.

The present invention also provides a composition containing the aforementioned saRNA and a pharmaceutically acceptable carrier. The pharmaceutically acceptable carrier is a liposome, a macromolecular polymer, or a polypeptide.

The present invention further provides the use of the saRNA or the composition in the preparation of a formulation for activating or upregulating the expression of the target gene. Preferably, the use of the saRNA or the composition in the preparation of a formulation is for treating a tumor or a benign proliferative lesion. Preferably, the tumor is a bladder cancer, a prostate cancer, a hepatocellular carcinoma, or a colorectal cancer.

### BRIEF DESCRIPTION OF THE DRAWINGS

FIG. 1 shows the sequence of the p21 gene promoter (SEQ ID NO:601), from −1000 bp upstream of the transcription start site (TSS) to +3 bp downstream of the TSS. The TSS is denoted by a curved arrow.

FIGS. 2A and 2B show saRNA hotspot regions in the p21 promoter as revealed by screening. Four hundred and thirty-nine (439) saRNAs were designed on the sequence of the p21 promoter shown in FIG. 1 and chemically synthesized. The resultant saRNAs were individually transfected into PC3 human prostate cancer cells. 72 hours after transfection, the mRNA levels of p21 were determined by using the QuantiGene 2.0 assay. FIG. 2A shows the fold change (Y axis) of p21 mRNA levels caused by each of the 439 saRNAs (X axis) relative to the control treatment (Mock). The saRNAs on the X axis are sorted according to their positions relative to the TSS of p21 (from the most upstream RAG-898 to the most downstream RAG1-177). Eight hotspot regions are identified and shown (grayish rectangular boxes). FIG. 2B shows the data of FIG. 2A by sorting the saRNAs by their activity in activating p21 mRNA expression in an ascending order. The dotted lines in FIG. 2A and FIG. 2B represent the position of a two-fold induction.

FIGS. 3A-3H show the activating effects of the saRNAs targeting the hotspot regions 1 to 8 on the p21 promoter (FIG. 3A: hotspot region 1; FIG. 3B: hotspot region 2;

FIG. 3C: hotspot region 3; FIG. 3D: hotspot region 4; FIG. 3E: hotspot region 5; FIG. 3F: hotspot region 6; FIG. 3G: hotspot region 7; FIG. 3H: hotspot region 8).

FIGS. 4A and 4B show the mRNA levels of p21 assessed by RT-qPCR for verifying the result obtained from the QuantiGene 2.0 assay. FIG. 4A shows the mRNA level of p21 determined by RT-qPCR. The 439 saRNAs were divided into four bins according to their activities in inducing p21 mRNA expression from the highest to the lowest, and 5 saRNAs were randomly selected from each bin and individually transfected into PC3 cells at a 10 nM concentration. 72 hours after transfection, mRNA was extracted for reverse transcription to obtain cDNA, and then the mRNA level of p21 was determined by RT-qPCR. FIG. 4B shows the correlation of relative p21 mRNA level induced by the saRNAs determined by QuantiGene 2.0 (X axis) and by RT-qPCR (Y axis).

FIGS. 5A-5C show the effect of saRNAs in inducing p21 mRNA expression and suppressing the proliferation of KU-7-Luc2-GFP cells. KU-7-Luc2-GFP cells were transfected with each of three saRNAs (RAG-431, RAG-553 and RAG-688) at a concentration of 10 nM for 72 hours. Mock samples were transfected in the absence of an oligonucleotide. Non-specific duplex (dsCon) served as a negative control. FIG. 5A shows the mRNA expression levels of p21 determined by RT-qPCR. FIG. 5B shows viability of saRNA treated cells as evaluated by the CCK-8 assay and plotted as percentages relative to the cell viability for control treatment (Mock). FIG. 5C shows representative cell images (100×) at the end of transfection.

FIGS. 6A-6C show the effect of the saRNAs in inducing p21 mRNA expression and suppressing the proliferation of HCT116 cells. HCT116 cells were transfected with each of three saRNAs (RAG-431, RAG-553 and RAG-688) at a concentration of 10 nM for 72 hours. FIG. 6A shows the mRNA expression levels of p21 determined by RT-qPCR. FIG. 6B shows the cell viability evaluated by the CCK-8 assay, and cell viability of saRNA-treated cells is plotted as percentages relative to the cell viability of the control treatment (Mock). FIG. 6C shows representative phase contrast cell images (100×) at the end of transfection.

FIGS. 7A-7C show the effect of the saRNAs in inducing p21 mRNA expression and suppressing the proliferation of HepG2 cells. HepG2 cells were transfected with each of three saRNAs (RAG-431, RAG-553 and RAG-688) at a concentration of 10 nM for 72 hours. FIG. 7A shows the mRNA expression levels of p21 determined by RT-qPCR. FIG. 7B shows the cell viability evaluated by the CCK-8 assay, and cell viability of saRNA-treated cells is plotted as percentages relative to the cell viability of the control treatment (Mock). FIG. 7C shows representative phase contrast cell images (100×) at the end of transfection.

FIG. 8 is a schematic diagram illustrating a p21 promoter region and a target sequence for saRNAs therein. A 40 bp region spanning nucleotides −332 to −292 relative to the TSS in the p21 promoter was targeted by a series of saRNA duplexes for gene activation. The sequences shown correspond to the sites targeted by their cognate duplexes. Note the defined promoter region contains 17 overlapping targeted sites (SEQ ID NOs:584-600) that shift by a single nucleotide each time skipping a short stretch of a polyadenosine repeat (underlined).

FIG. 9 shows the gene induction activities of saRNAs targeting the p21 promoter. KU-7-luc2-GFP cells were transfected at 10 nM concentrations of the indicated saRNAs for 72 hours. Mock samples were transfected in the absence of an oligonucleotide. Non-specific duplex (dsCon) served as a negative control. Relative expression was determined by RT-qPCR (mean±SD). Expression levels of p21 were normalized to that of GAPDH. Statistical significance was determined by one-way ANOVA and Tukey's multiple comparisons test (***p<0.001 versus mock).

FIG. 10 is a schematic diagram showing sequences and structures of saRNA variants (from Rag1-0 to Rag1-20) (SEQ ID NOs:21-24,37-68). Mispaired bases are represented by colons. Boldfaces represent mutated nucleotides in saRNA variants compared to the original Rag1-0.

FIGS. 11A-11C show the activity of the Rag1 variants (Rag1-0 to Rag1-20) in inducing p21 expression in human cancer cell lines. KU-7-Luc2-GFP (FIG. 11A), PC3 (FIG. 11B), and Bel-7402 (FIG. 11C) cells were transfected with 10 nM of the indicated Rag1 duplex variants for 72 hours. Mock samples were transfected in the absence of an oligonucleotide. Non-specific duplex (dsCon) served as a negative control. Relative p21 mRNA expression was determined by RT-qPCR (mean±SD). Expression levels of p21 were normalized to that of GAPDH. Statistical significance was determined by one-way ANOVA and Tukey's multiple comparisons test (*P<0.5; **P<0.01; ***p<0.001 versus Mock).

FIGS. 12A-12C show the suppressing effect of the saRNAs (Rag1-0 to Rag1-20) on the proliferation of human cancer cell lines. Cell viabilities were quantitatively assayed by the CCK-8 method. Statistical significance was determined by one-way ANOVA and Tukey's multiple comparisons test (*P<0.5; **P<0.01; ***p<0.001 versus Mock).

FIG. 13 is a schematic diagram for the sequences and structures of duplex saRNAs Rag1-21 to Rag1-44 (SEQ ID NOs:38, 40, 69-88). Mispaired bases in duplexes are represented by colons, and boldfaces represent altered nucleotides or structure in the variants.

FIGS. 14A-14E show the activities of the saRNAs (Rag1-21 to Rag1-28) in inducing p21 expression in human cancer cell lines. KU-7-Luc2-GFP (FIG. 14A), UM-UC-3 (FIG. 14B), T24 (FIG. 14C), J82 (FIG. 14D) and Bel-7402 (FIG. 14E) cells were transfected with 10 nM of the indicated saRNA variants for 72 hours. Mock samples were transfected in the absence of an oligonucleotide. Non-specific duplex (dsCon) served as a negative control. Relative p21 mRNA expression was determined by RT-qPCR (mean±SD). Expression levels of p21 were normalized to that of GAPDH. Statistical significance was determined by one-way ANOVA and Tukey's multiple comparisons test (*P<0.5; **P<0.01; ***p<0.001 versus Mock).

FIGS. 15A-15E show the suppressing effect of the saRNAs (Rag1-21 to Rag1-28) on the proliferation of human cancer cell lines. KU-7-Luc2-GFP (FIG. 15A), UM-UC-3 (FIG. 15B), T24 (FIG. 15C), J82 (FIG. 15D) and Bel-7402 (FIG. 15E) cells were transfected with 10 nM of the indicated saRNA variants for 72 hours. Mock samples were transfected in the absence of an oligonucleotide. Non-specific duplex (dsCon) served as a negative control. Cell viabilities were quantitatively assayed by the CCK-8 method. Statistical significance was determined by one-way ANOVA and Tukey's multiple comparisons test (*P<0.5; **P<0.01; ***p<0.001 versus Mock).

FIGS. 16A-16E show the activity of saRNAs (Rag1-29 to Rag1-44) in inducing p21 expression in human cancer cell lines. KU-7-Luc2-GFP (FIG. 16A), UM-UC-3 (FIG. 16B), T24 (FIG. 16C), J82 (FIG. 16D), and PC3 (FIG. 16E) cells were transfected with 10 nM of the indicated saRNA variants for 72 hours. Mock samples were transfected in the absence of an oligonucleotide. Non-specific duplex (dsCon) served as a negative control. Relative p21 mRNA expression was determined by RT-qPCR (mean±SD). Expression levels of p21 were normalized to that of GAPDH. Statistical significance was determined by one-way ANOVA and Tukey's multiple comparisons test (*P<0.5; **P<0.01; ***p<0.001 versus Mock).

FIGS. 17A-17E show the suppressing effect of the saRNAs (Rag1-29 to Rag1-44) on the proliferation of human cancer cell lines. KU-7-Luc2-GFP (FIG. 17A), UM-UC-3 (FIG. 17B), T24 (FIG. 17C), J82 (FIG. 17D), and PC3 (FIG. 17E) cells were transfected with 10 nM of the indicated saRNA variants for 72 hours. Mock samples were transfected in the absence of an oligonucleotide. Non-specific duplex (dsCon) served as a negative control. Cell viability was quantitatively assayed by the CCK-8 method. Statistical significance was determined by one-way ANOVA and Tukey's multiple comparisons test (*P<0.5; **P<0.01; ***p<0.001 versus Mock).

FIG. 18 is a schematic diagram for p21 saRNA duplex sequences (SEQ ID NOs: 211, 212, 315, 316, 375, 376, 385, 386, 557-563). Mispaired bases are represented by colons, and boldfaces represent altered nucleotides or structure in saRNA variants.

FIGS. 19A-19D show gene induction activity of saRNAs targeting the p21 promoter. KU-7-Luc2-GFP cells were transfected with 10 nM of the indicated saRNA variants for 72 hours. Mock samples were transfected in the absence of an oligonucleotide. Non-specific duplex (dsCon) served as a negative control. Relative p21 mRNA expression was determined by RT-qPCR (mean±SD). Expression levels of p21 were normalized to that of GAPDH.

FIG. 20 is a schematic diagram of saRNA duplex sequences targeting the promoter of KLF4, NKX3-1, and VEGFA genes (SEQ ID NOs: 564-577). Mispaired bases are represented by colons, and boldfaces represent altered nucleotides or structures in saRNA variants.

FIGS. 21A-21C show the gene induction activities of saRNAs targeting the KLF4 promoter. PC3 (FIG. 21A), KU-7-Luc2-GFP (FIG. 21B), and Bel-7402 (FIG. 21C) cells were transfected with the indicated saRNAs individually at the indicated concentration for 72 hours. Transfected samples containing no oligonucleotide were adopted as blank controls (Mock), and non-specific duplex (dsCon) transfected samples were adopted as negative controls. Relative KLF4 mRNA expression was determined by RT-qPCR (mean±SD). Expression levels of KLF4 were normalized to that of GAPDH.

FIGS. 22A and 22B show the gene induction activities of saRNAs targeting the NKX3-1 promoter. PC3 (FIG. 22A) and Bel-7402 (FIG. 22B) cells were transfected with the indicated saRNAs individually at the indicated concentration for 72 hours. Transfected samples containing no oligonucleotide were adopted as blank controls (Mock), and non-specific duplex (dsCon) transfected samples were adopted as negative controls. Relative NKX3-1 mRNA expression was determined by RT-qPCR (mean±SD). Expression levels of NKX3-1 were normalized to that of GAPDH.

FIGS. 23A-23C show gene induction activity of saRNAs targeting the VEGFA promoter. HeLa (FIG. 23A), COS-1 (FIG. 23B), and ARPE-19 (FIG. 23C) cells were transfected with the indicated saRNAs individually at the indicated concentration for 72 hours. Transfected samples containing no oligonucleotide were adopted as blank controls (Mock), and non-specific duplex (dsCon) transfected samples were adopted as negative controls. Relative VEGFA mRNA expression was determined by RT-qPCR (mean±SD). Expression levels of VEGFA were normalized to that of GAPDH.

FIG. 24 shows the suppression of in vivo tumor growth by p21 saRNA. The indicated saRNA was intratumorally injected at a dose of 1 mg/kg, and the volume of tumor was recorded. The arrows indicate the time points of injections.

### DETAILED DESCRIPTION OF THE INVENTION

The present invention is further described hereinafter by specific description.

Unless otherwise defined, all the technological and scientific terms used therein have the same meanings as those generally understood by those of ordinary skill in the art covering the present invention.

In the present application, singular forms, such as “a” and “this”, include plural objects, unless otherwise specified clearly in the context.

### Definition

The term “complementary” as used herein refers to the capability of forming base pairs between two oligonucleotide strands. The base pairs are generally formed by hydrogen bonds between nucleotide units in the antiparallel oligonucleotide strands. The bases of the complementary oligonucleotide strands can be paired in the Watson-Crick manner (such as A to T, A to U, and C to G) or in any other manners allowing the formation of a duplex (such as Hoogsteen or reverse Hoogsteen base pair). “100% pairing” refers to 100% complementarity, that is, all the nucleotide units of the two strands are bound by hydrogen bonds.

“Complete complementarity” or “100% complementarity” means that each nucleotide unit from the first oligonucleotide strand can form a hydrogen bond with the second oligonucleotide strand in the double-stranded region of the double-stranded oligonucleotide molecule, with no base pair being mispaired. “Incomplete complementarity” means that not all the nucleotide units of the two strands are bound with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. “Substantial complementarity” refers to more than about 79%, about 80%, about 85%, about 90%, or about 95% complementarity.

The term “oligonucleotide” as used herein refers to polymers of nucleotides, and includes, but is not limited to, single-stranded or double-stranded molecules of DNA, RNA, or DNA/RNA hybrid, oligonucleotide strands containing regularly and irregularly alternating deoxyribonucleotide portions and ribonucleotide portions, as well as modified and naturally or unnaturally existed frameworks for such oligonucleotides. The oligonucleotide for activating target gene transcription described in the present invention is a saRNA.

The term “oligoribonucleotide” as used herein refers to an oligonucleotide containing two or more modified or unmodified ribonucleotides and/or analogues thereof.

The terms “oligonucleotide strand” and “oligonucleotide sequence” as used herein can be used interchangeably, referring to a generic term for short nucleotide sequences having less than 30 bases (including deoxyribonucleic acid (DNA) or ribonucleic acid (RNA)). In the present invention, the length of an oligonucleotide strand can be any length from 17 to 30 nucleotides.

The term “gene” as used herein refers to all nucleotide sequences required to code a polypeptide chain or to transcribe a functional RNA. “Gene” can be an endogenous or fully or partially recombinant gene for a host cell (for example, because an exogenous oligonucleotide and a coding sequence for coding a promoter are introduced into a host cell, or a heterogeneous promoter adjacent to an endogenous coding sequence is introduced into a host cell). For example, the term “gene” includes a nucleic acid sequence composed of exons and introns. Protein-coding sequences are, for example, sequences contained within exons in an open reading frame between an initiation codon and a termination codon, and as used herein, “gene” can comprise a gene regulatory sequence, such as a promoter, an enhancer, and all other sequences known in the art for controlling the transcription, expression or activity of another gene, no matter whether the gene contains a coding sequence or a non-coding sequence. In one case, for example, “gene” can be used to describe a functional nucleic acid containing a regulatory sequence such as a promoter or an enhancer. The expression of a recombinant gene can be controlled by one or more types of heterogeneous regulatory sequences.

The term “target gene” as used herein can refer to nucleic acid sequences, transgenes, viral or bacterial sequences, chromosomes or extrachromosomal genes that are naturally present in organisms, and/or can be transiently or stably transfected or incorporated into cells and/or chromatins thereof. The target gene can be a protein-coding gene or a non-protein-coding gene (such as a microRNA gene and a long non-coding RNA gene). The target gene generally contains a promoter sequence, and the positive regulation for the target gene can be achieved by designing a saRNA having sequence homology with the promoter sequence, characterized as the upregulation of expression of the target gene. “Sequence of a target gene promoter” refers to a non-coding sequence of the target gene, and the reference of the sequence of a target gene promoter in the phrase “complementary with the sequence of a target gene promoter” of the present invention means a coding strand of the sequence, also known as a non-template strand, i.e. a nucleic acid sequence having the same sequence as the coding sequence of the gene. “Target sequence” refers to a sequence fragment in the target gene promoter, which is homologous or complementary with a sense oligonucleotide strand or an antisense oligonucleotide strand of a saRNA.

As used herein, the term “first oligonucleotide strand” can be a sense strand or an antisense strand. The sense strand of a saRNA refers to an oligonucleotide strand having homology with the coding strand of the promoter DNA sequence of the target gene in the saRNA duplex. The antisense strand refers to an oligonucleotide strand complementary with the sense strand in the saRNA duplex.

As used herein, the term “second oligonucleotide strand” can also be a sense strand or an antisense strand. If the first oligonucleotide strand is a sense strand, the second oligonucleotide strand is an antisense strand; and if the first oligonucleotide strand is an antisense strand, the second oligonucleotide strand is a sense strand.

The term “coding strand” as used herein refers to a DNA strand in the target gene which is not used for transcription of the gene, and the nucleotide sequence of this strand is the same as that of a RNA produced from transcription (in the RNA, T in DNA is replaced by U). The coding strand of the double-stranded DNA sequence of the target gene promoter described in the present invention refers to a promoter sequence on the same DNA strand as the DNA coding strand of the target gene.

The term “template strand” as used herein refers to the other strand complementary with the coding strand in the double-stranded DNA of the target gene, i.e. the strand that, as a template, can be transcribed into RNA, and this strand is complementary with the transcribed RNA (A to U and G to C). In the process of transcription, RNA polymerase is bound with the template strand, moves along the 3′→5′ direction of the template strand, and catalyzes the synthesis of the RNA in the 5′→3′ direction. The template strand of the double-stranded DNA sequence of the target gene promoter described in the present invention refers to a promoter sequence on the same DNA strand as the DNA template strand of the target gene.

The term “promoter” as used herein refers to a nucleic acid sequence, which encodes no proteins and plays a regulatory role for the transcription of a protein-coding or RNA-coding nucleic acid sequence by associating with them spatially. Generally, a eukaryotic promoter contains 100 to 5,000 base pairs, although this length range is not intended to limit the term of “promoter” as used herein. Although the promoter sequence is generally located at the 5′ terminus of a protein-coding or RNA-coding sequence, it also exists in exon and intron sequences.

The term “transcription start site” as used herein refers to a nucleotide marking the transcription start on the template strand of a gene. The transcription start site can appear on the template strand of the promoter region. A gene can have more than one transcription start sites.

The term “sequence identity” or “sequence homology” as used herein means that one oligonucleotide strand (sense or antisense) of a saRNA has at least 80% similarity with a region on the coding strand or template strand of the promoter sequence of a target gene.

The term “overhang” as used herein refers to non-base-paired nucleotides at the terminus (5′ or 3′) of an oligonucleotide strand, which is formed by one strand extending out of the other strand in a duplex oligonucleotide. A single-stranded region extending out of the 3′ terminus and/or 5′ terminus of a duplex is referred to as an overhang.

The terms “natural overhang” and “natural nucleotide overhang” as used herein can be used interchangeably, and refer to an overhang, at the 5′ or 3′ terminus of the sense or antisense strand of an saRNA, that is derived from or homologous to its target sequence.

As used herein, the terms “gene activation” or “activating gene expression” can be used interchangeably, and means an increase or upregulation in transcription, translation, expression or activity of a certain nucleic acid as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “small activating RNA” and “saRNA” can be used interchangeably and refer to a ribonucleic acid molecule that can upregulate target gene expression. It can be a double-stranded nucleic acid molecule composed of a first nucleic acid strand containing a ribonucleotide sequence with sequence homology with the non-coding nucleic acid sequence (such as a promoter and an enhancer) of a target gene and a second nucleic acid strand containing a nucleotide sequence complementary with the first strand. The saRNA can also be comprised of a synthesized or vector-expressed single-stranded RNA molecule that can form a hairpin structure by two complementary regions within the molecule, wherein the first region contains a ribonucleotide sequence having sequence homology with the target sequence of a promoter of a gene, and a ribonucleotide sequence contained in the second region is complementary with the first region. The length of the duplex region of the saRNA molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45 or about 50 base pairs. In addition, the terms “small activating RNA” and “saRNA” also contain nucleic acids other than the ribonucleotide, including, but not limited to, modified nucleotides or analogues.

As used herein, the term “hotspot” refers to a promoter region of a gene where targets for functional saRNAs are enriched. A hotspot is defined as a sequence region containing targets for at least 10 saRNAs, wherein at least 60% of the saRNAs can induce a 1.5-fold or more change in the mRNA expression of the gene.

As used herein, the term “synthesis” refers to the method for synthesis of an oligonucleotide, including any method allowing RNA synthesis, such as chemical synthesis, in vitro transcription, and/or vector-based expression.

As used herein, the term “p21” refers to the p21^{WAF1/CIP1} gene, also known as the CDKN1A gene. As a cyclin-dependent kinase (CDK) inhibitor, p21 is an important tumor suppressor gene. Overexpression of p21 or activation of endogenous p21 transcription has been shown to inhibit the growth of cultured tumor cells and tumors in vivo.

Materials and Methods

Cell Culture and Transfection

Cell lines RT4, KU-7, T24, and HT-1197 were cultured in the modified McCoy's 5A medium (Gibco); cell lines J82, TCCSUP, and UM-UC-3 were cultured in the MEM medium (Gibco); and cell lines 5637, PC3, and Bel-7402 were cultured in the RPMI-1640 medium (Gibco). All the media contained 10% bovine calf serum (Sigma-Aldrich) and 1% penicillin/streptomycin (Gibco). The cells were cultured at 5% CO₂ and 37° C. saRNAs were transfected into cells using RNAiMax (Invitrogen, Carlsbad, Calif.) according to the instructions provided by the manufacturer at the concentration of 10 nM (unless otherwise specified). All the saRNA sequences are listed in Table 1.

TABLE 1
Strand sequences and duplex compositions

| Title | Sequence No. | Sequence (5′-3′) | Length |
|---|---|---|---|
| dsCon | SEQ ID NO 1 | ACUACUGAGUGACAGUAGA[dT][dT] | 21 nt |
| | SEQ ID NO 2 | UCUACUGUCACUCAGUAGU[dT][dT] | 21 nt |
| P21-332 | SEQ ID NO 3 | CUGGAGAGUGCCAACUCAU[dT][dT] | 21 nt |
| | SEQ ID NO 4 | AUGAGUUGGCACUCUCCAG[dT][dT] | 21 nt |
| P21-331 | SEQ ID NO 5 | UGGAGAGUGCCAACUCAUU[dT][dT] | 21 nt |
| | SEQ ID NO 6 | AAUGAGUUGGCACUCUCCA[dT][dT] | 21 nt |
| P21-330 | SEQ ID NO 7 | GGAGAGUGCCAACUCAUUC[dT][dT] | 21 nt |
| | SEQ ID NO 8 | GAAUGAGUUGGCACUCUCC[dT][dT] | 21 nt |
| P21-329 | SEQ ID NO 9 | GAGAGUGCCAACUCAUUCU[dT][dT] | 21 nt |
| | SEQ ID NO 10 | AGAAUGAGUUGGCACUCUC[dT][dT] | 21 nt |
| P21-328 | SEQ ID NO 11 | AGAGUGCCAACUCAUUCUC[dT][dT] | 21 nt |
| | SEQ ID NO 12 | GAGAAUGAGUUGGCACUCU[dT][dT] | 21 nt |
| P21-327 | SEQ ID NO 13 | GAGUGCCAACUCAUUCUCC[dT][dT] | 21 nt |
| | SEQ ID NO 14 | GGAGAAUGAGUUGGCACUC[dT][dT] | 21 nt |
| P21-326 | SEQ ID NO 15 | AGUGCCAACUCAUUCUCCA[dT][dT] | 21 nt |
| | SEQ ID NO 16 | UGGAGAAUGAGUUGGCACU[dT][dT] | 21 nt |
| P21-325 | SEQ ID NO 17 | GUGCCAACUCAUUCUCCAA[dT][dT] | 21 nt |
| | SEQ ID NO 18 | UUGGAGAAUGAGUUGGCAC[dT][dT] | 21 nt |
| P21-324 | SEQ ID NO 19 | UGCCAACUCAUUCUCCAAG[dT][dT] | 21 nt |
| | SEQ ID NO 20 | CUUGGAGAAUGAGUUGGCA[dT][dT] | 21 nt |
| P21-323 | SEQ ID NO 21 | GCCAACUCAUUCUCCAAGU[dT][dT] | 21 nt |
| | SEQ ID NO 22 | ACUUGGAGAAUGAGUUGGC[dT][dT] | 21 nt |
| P21-322 | SEQ ID NO 23 | CCAACUCAUUCUCCAAGUA[dT][dT] | 21 nt |
| | SEQ ID NO 24 | UACUUGGAGAAUGAGUUGG[dT][dT] | 21 nt |
| P21-321 | SEQ ID NO 25 | CAACUCAUUCUCCAAGUAA[dT][dT] | 21 nt |
| | SEQ ID NO 26 | UUACUUGGAGAAUGAGUUG[dT][dT] | 21 nt |
| P21-301 | SEQ ID NO 27 | AAAAGCCAGAUUUGUGGCU[dT][dT] | 21 nt |
| | SEQ ID NO 28 | AGCCACAAAUCUGGCUUUU[dT][dT] | 21 nt |
| P21-300 | SEQ ID NO 29 | AAAGCCAGAUUUGUGGCUC[dT][dT] | 21 nt |
| | SEQ ID NO 30 | GAGCCACAAAUCUGGCUUU[dT][dT] | 21 nt |
| P21-299 | SEQ ID NO 31 | AAGCCAGAUUUGUGGCUCA[dT][dT] | 21 nt |
| | SEQ ID NO 32 | UGAGCCACAAAUCUGGCUU[dT][dT] | 21 nt |
| P21-298 | SEQ ID NO 33 | AGCCAGAUUUGUGGCUCAC[dT][dT] | 21 nt |
| | SEQ ID NO 34 | GUGAGCCACAAAUCUGGCU[dT][dT] | 21 nt |
| P21-297 | SEQ ID NO 35 | GCCAGAUUUGUGGCUCACU[dT][dT] | 21 nt |
| | SEQ ID NO 36 | AGUGAGCCACAAAUCUGGC[dT][dT] | 21 nt |
| Rag1-0 | SEQ ID NO 23 | CCAACUCAUUCUCCAAGUA[dT][dT] | 21 nt |
| | SEQ ID NO 24 | UACUUGGAGAAUGAGUUGG[dT][dT] | 21 nt |
| Rag1-1 | SEQ ID NO 37 | CCAACUCAUUCUCCAAGUAAA | 21 nt |
| | SEQ ID NO 38 | UACUUGGAGAAUGAGUUGGCA | 21 nt |
| Rag1-2 | SEQ ID NO 39 | CCAACUCAUUCUCCAAGUAUU | 21 nt |
| | SEQ ID NO 40 | UACUUGGAGAAUGAGUUGGUU | 21 nt |
| Rag1-3 | SEQ ID NO 41 | CCAACUCAUUCUCCAAGUAA[dT][dT] | 22 nt |
| | SEQ ID NO 42 | UUACUUGGAGAAUGAGUUGG[dT][dT] | 22 nt |
| Rag1-4 | SEQ ID NO 43 | CCAACUCAUUCUCCAAGUAAA[dT][dT] | 23 nt |
| | SEQ ID NO 44 | UUUACUUGGAGAAUGAGUUGG[dT][dT] | 23 nt |
| Rag1-5 | SEQ ID NO 45 | CCAACUCAUUCUCCAAGUAAAA[dT][dT] | 24 nt |
| | SEQ ID NO 46 | UUUUACUUGGAGAAUGAGUUGG[dT][dT] | 24 nt |
| Rag1-6 | SEQ ID NO 47 | CCAACUCAUUCUCCAAGUAAAAA[dT][dT] | 25 nt |
| | SEQ ID NO 48 | UUUUUACUUGGAGAAUGAGUUGG[dT][dT] | 25 nt |
| Rag1-7 | SEQ ID NO 49 | CCAACUCAUUCUCCAAGUAAAAAA[dT][dT] | 26 nt |
| | SEQ ID NO 50 | UUUUUUACUUGGAGAAUGAGUUGG[dT][dT] | 26 nt |
| Rag1-8 | SEQ ID NO 21 | GCCAACUCAUUCUCCAAGU[dT][dT] | 21 nt |
| | SEQ ID NO 22 | ACUUGGAGAAUGAGUUGGC[dT][dT] | 21 nt |
| Rag1-9 | SEQ ID NO 51 | GCCAACUCAUUCUCCAAGUAA | 21 nt |
| | SEQ ID NO 52 | ACUUGGAGAAUGAGUUGGCAC | 21 nt |
| Rag1-10 | SEQ ID NO 53 | GCCAACUCAUUCUCCAAGUA[dT][dT] | 22 nt |
| | SEQ ID NO 54 | UACUUGGAGAAUGAGUUGGC[dT][dT] | 22 nt |
| Rag1-11 | SEQ ID NO 55 | GCCAACUCAUUCUCCAAGUAA[dT][dT] | 23 nt |
| | SEQ ID NO 56 | UUACUUGGAGAAUGAGUUGGC[dT][dT] | 23 nt |
| Rag1-12 | SEQ ID NO 57 | GCCAACUCAUUCUCCAAGUAAA[dT][dT] | 24 nt |
| | SEQ ID NO 58 | UUUACUUGGAGAAUGAGUUGGC[dT][dT] | 24 nt |
| Rag1-13 | SEQ ID NO 59 | GCCAACUCAUUCUCCAAGUAAAA[dT][dT] | 25 nt |
| | SEQ ID NO 60 | UUUUACUUGGAGAAUGAGUUGGC[dT][dT] | 25 nt |
| Rag1-14 | SEQ ID NO 61 | GCCAACUCAUUCUCCAAGUAAAAA[dT][dT] | 26 nt |
| | SEQ ID NO 62 | UUUUUACUUGGAGAAUGAGUUGGC[dT][dT] | 26 nt |
| Rag1-15 | SEQ ID NO 63 | CCAACUCAUUCUCCAAGUU[dT][dT] | 21 nt |
| | SEQ ID NO 24 | UACUUGGAGAAUGAGUUGG[dT][dT] | 21 nt |
| Rag1-16 | SEQ ID NO 64 | CCAACUCAUUCUCCAAGUC[dT][dT] | 21 nt |
| | SEQ ID NO 24 | UACUUGGAGAAUGAGUUGG[dT][dT] | 21 nt |
| Rag1-17 | SEQ ID NO 65 | CCAACUCAUUCUCCAAGAU[dT][dT] | 21 nt |
| | SEQ ID NO 24 | UACUUGGAGAAUGAGUUGG[dT][dT] | 21 nt |
| Rag1-18 | SEQ ID NO 66 | CCAACUCAUUCUCCAAGUA | 19 nt |
| | SEQ ID NO 24 | UACUUGGAGAAUGAGUUGG[dT][dT] | 21 nt |
| Rag1-19 | SEQ ID NO 67 | CAACUCAUUCUCCAAGUA | 18 nt |
| | SEQ ID NO 24 | UACUUGGAGAAUGAGUUGG[dT][dT] | 21 nt |
| Rag1-20 | SEQ ID NO 68 | CAACUCAUUCUCCAAGUA[dT][dT] | 20 nt |
| | SEQ ID NO 24 | UACUUGGAGAAUGAGUUGG[dT][dT] | 21 nt |
| Rag1-21 | SEQ ID NO 69 | GCCAACUCAUUCUCCAAGUAUU | 22 nt |
| | SEQ ID NO 70 | UACUUGGAGAAUGAGUUGGCUU | 22 nt |
| Rag1-22 | SEQ ID NO 71 | GCCAACUCAUUCUCCAAGUAAA | 22 nt |
| | SEQ ID NO 72 | UACUUGGAGAAUGAGUUGGCAC | 22 nt |
| Rag1-23 | SEQ ID NO 73 | GCCAACUCAUUCUCCAAGUCUU | 22 nt |
| | SEQ ID NO 70 | UACUUGGAGAAUGAGUUGGCUU | 22 nt |
| Rag1-24 | SEQ ID NO 74 | CAACUCAUUCUCCAAGUCUU | 20 nt |
| | SEQ ID NO 70 | UACUUGGAGAAUGAGUUGGCUU | 22 nt |
| Rag1-25 | SEQ ID NO 75 | CCAACUCAUUCUCCAAGUCUU | 21 nt |
| | SEQ ID NO 40 | UACUUGGAGAAUGAGUUGGUU | 21 nt |
| Rag1-26 | SEQ ID NO 76 | CCAACUCAUUCUCCAAGUCAA | 21 nt |
| | SEQ ID NO 38 | UACUUGGAGAAUGAGUUGGCA | 21 nt |
| Rag1-27 | SEQ ID NO 74 | CAACUCAUUCUCCAAGUCUU | 20 nt |
| | SEQ ID NO 40 | UACUUGGAGAAUGAGUUGGUU | 21 nt |
| Rag1-28 | SEQ ID NO 77 | CAACUCAUUCUCCAAGUCAA | 20 nt |
| | SEQ ID NO 38 | UACUUGGAGAAUGAGUUGGCA | 21 nt |
| Rag1-29 | SEQ ID NO 78 | GCCAACUCAUUCUCCAAGUCAA | 22 nt |
| | SEQ ID NO 79 | UACUUGGAGAAUGAGUUGGC | 20 nt |
| Rag1-30 | SEQ ID NO 74 | CAACUCAUUCUCCAAGUCUU | 20 nt |
| | SEQ ID NO 38 | UACUUGGAGAAUGAGUUGGCA | 21 nt |
| Rag1-31 | SEQ ID NO 80 | CAACUCAUUCUCCAAGUC | 18 nt |
| | SEQ ID NO 38 | UACUUGGAGAAUGAGUUGGCA | 21 nt |
| Rag1-32 | SEQ ID NO 78 | GCCAACUCAUUCUCCAAGUCAA | 22 nt |
| | SEQ ID NO 72 | UACUUGGAGAAUGAGUUGGCAC | 22 nt |
| Rag1-33 | SEQ ID NO 81 | GCCAACUCAUUCUCCAAGUGAA | 22 nt |
| | SEQ ID NO 72 | UACUUGGAGAAUGAGUUGGCAC | 22 nt |
| Rag1-34 | SEQ ID NO 76 | CCAACUCAUUCUCCAAGUCAA | 21 nt |
| | SEQ ID NO 72 | UACUUGGAGAAUGAGUUGGCAC | 22 nt |
| Rag1-35 | SEQ ID NO 82 | GCCAACUCAUUCUCCAAGUC | 20 nt |
| | SEQ ID NO 72 | UACUUGGAGAAUGAGUUGGCAC | 22 nt |
| Rag1-36 | SEQ ID NO 83 | CCAACUCAUUCUCCAAGUC | 19 nt |
| | SEQ ID NO 72 | UACUUGGAGAAUGAGUUGGCAC | 22 nt |
| Rag1-37 | SEQ ID NO 84 | CCAACUCAUUCUCCAAGU | 18 nt |
| | SEQ ID NO 72 | UACUUGGAGAAUGAGUUGGCAC | 22 nt |
| Rag1-38 | SEQ ID NO 74 | CAACUCAUUCUCCAAGUCUU | 20 nt |
| | SEQ ID NO 85 | UUUACUUGGAGAAUGAGUUGG | 21 nt |
| Rag1-39 | SEQ ID NO 86 | CAACUCAUUCUCCAAGUGAA | 20 nt |
| | SEQ ID NO 38 | UACUUGGAGAAUGAGUUGGCA | 21 nt |
| Rag1-40 | SEQ ID NO 83 | CCAACUCAUUCUCCAAGUC | 19 nt |
| | SEQ ID NO 38 | UACUUGGAGAAUGAGUUGGCA | 21 nt |
| Rag1-41 | SEQ ID NO 87 | CCAACUCAUUCUCCAAGUG | 19 nt |
| | SEQ ID NO 38 | UACUUGGAGAAUGAGUUGGCA | 21 nt |
| Ragl-42 | SEQ ID NO 84 | CCAACUCAUUCUCCAAGU | 18 nt |
| | SEQ ID NO 38 | UACUUGGAGAAUGAGUUGGCA | 21 nt |
| Rag1-43 | SEQ ID NO 80 | CAACUCAUUCUCCAAGUC | 18 nt |
| | SEQ ID NO 38 | UACUUGGAGAAUGAGUUGGCA | 21 nt |
| Rag1-44 | SEQ ID NO 88 | CAACUCAUUCUCCAAGU | 17 nt |
| | SEQ ID NO 38 | UACUUGGAGAAUGAGUUGGCA | 21 nt |
| Rag-431-0 | SEQ ID NO 211 | CCUCCUGAUCUUUUCAGCU[dT][dT] | 21 nt |
| | SEQ ID NO 212 | AGCUGAAAAGAUCAGGAGG[dT][dT] | 21 nt |
| Rag-431-1 | SEQ ID NO 557 | CCUCCUGAUCUUUUCAGCC | 19 nt |
| | SEQ ID NO 558 | AGCUGAAAAGAUCAGGAGGAU | 21 nt |
| Rag-431-3 | SEQ ID NO 557 | CCUCCUGAUCUUUUCAGCC | 19 nt |
| | SEQ ID NO 212 | AGCUGAAAAGAUCAGGAGG[dT][dT] | 21 nt |
| Rag-553-0 | SEQ ID NO 315 | UCUGGGAGAGGUGACCUAG[dT][dT] | 21 nt |
| | SEQ ID NO 316 | CUAGGUCACCUCUCCCAGA[dT][dT] | 21 nt |
| Rag-553-1 | SEQ ID NO 559 | UCUGGGAGAGGUGACCUAA | 19 nt |
| | SEQ ID NO 560 | CUAGGUCACCUCUCCCAGAAG | 21 nt |
| Rag-553-3 | SEQ ID NO 559 | UCUGGGAGAGGUGACCUAA | 19 nt |
| | SEQ ID NO 316 | CUAGGUCACCUCUCCCAGA[dT][dT] | 21 nt |
| Rag-688-0 | SEQ ID NO: 375 | AAGCAUGUGACAAUCAACA[dT][dT] | 21 nt |
| | SEQ ID NO: 376 | UGUUGAUUGUCACAUGCUU[dT][dT] | 21 nt |
| Rag-688-1 | SEQ ID NO 560 | AAGCAUGUGACAAUCAACC | 19 nt |
| | SEQ ID NO 561 | UGUUGAUUGUCACAUGCUUCC | 21 nt |
| Rag-688-3 | SEQ ID NO 560 | AAGCAUGUGACAAUCAACC | 19 nt |
| | SEQ ID NO: 376 | UGUUGAUUGUCACAUGCUU[dT][dT] | 21 nt |
| Rag-693-0 | SEQ ID NO: 385 | CCCGGAAGCAUGUGACAAU[dT][dT] | 21 nt |
| | SEQ ID NO: 386 | AUUGUCACAUGCUUCCGGG[dT][dT] | 21 nt |
| Rag-693-1 | SEQ ID NO 562 | CCCGGAAGCAUGUGACAAC | 19 nt |
| | SEQ ID NO 563 | AUUGUCACAUGCUUCCGGGAA | 21 nt |
| Rag-693-3 | SEQ ID NO 562 | CCCGGAAGCAUGUGACAAC | 19 nt |
| | SEQ ID NO: 386 | AUUGUCACAUGCUUCCGGG[dT][dT] | 21 nt |
| KLF4-0 | SEQ ID NO 564 | GAACCCAGGGAGCCGACAA[dT][dT] | 21 nt |
| | SEQ ID NO 565 | UUGUCGGCUCCCUGGGUUC[dT][dT] | 21 nt |
| KLF4-1 | SEQ ID NO 566 | GAACCCAGGGAGCCGACAC | 19 nt |
| | SEQ ID NO 567 | UUGUCGGCUCCCUGGGUUCUU | 21 nt |
| NKX3-1-0 | SEQ ID NO 568 | GACGGUCCUGAAGAGCUAA[dT][dT] | 21 nt |
| | SEQ ID NO 569 | UUAGCUCUUCAGGACCGUC[dT][dT] | 21 nt |
| NKX3-1-1 | SEQ ID NO 570 | GACGGUCCUGAAGAGCUAC | 19 nt |
| | SEQ ID NO 571 | UUAGCUCUUCAGGACCGUCAG | 21 nt |
| Nkx3-1-2 | SEQ ID NO 572 | GACGGUCCUGAAGAGCUAG | 19 nt |
| | SEQ ID NO 571 | UUAGCUCUUCAGGACCGUCAG | 21 nt |
| NKX3-1-3 | SEQ ID NO 573 | GACGGUCCUGAAGAGCUAU | 19 nt |
| | SEQ ID NO 571 | UUAGCUCUUCAGGACCGUCAG | 21 nt |
| VEGF-0 | SEQ ID NO 574 | GCAACUCCAGUCCCAAAUA[dT][dT] | 21 nt |
| | SEQ ID NO 575 | UAUUUGGGACUGGAGUUGC[dT][dT] | 20 nt |
| VEGF-1 | SEQ ID NO 576 | GCAACUCCAGUCCCAAAUC | 19 nt |
| | SEQ ID NO 577 | UAUUUGGGACUGGAGUUGCUU | 21 nt |

RNA Isolation and Reverse Transcription-Polymerase Chain Reaction (RT-PCR)

Cells were seeded into 6-well plates with 2-3×10⁵ cells/well and were reverse transfected with oligonucleotide duplexes. At the end of the transfection, total cellular RNA was isolated using an RNeasy Plus Mini kit (Qiagen; Hilden, Germany) according to its manual. The resultant RNA (1 μg) was reverse transcribed into cDNA by using a PrimeScript RT kit containing gDNA Eraser (Takara, Shlga, Japan). The resultant cDNA was amplified in an ABI 7500 Fast Real-time PCR System (Applied Biosystems; Foster City, Calif.) using SYBR Premix Ex Taq II (Takara, Shlga, Japan) reagents and target gene specific primers. The reaction conditions were: 95° C. for 3 seconds, 60° C. for 30 seconds, and 40 cycles. Amplification of GAPDH served as an internal control. All primer sequences are listed in Table 2.

TABLE 2
Primer sequences for RT-qPCR assay

| Primer Title | Sequence No. | Sequence (5′-3′) |
|---|---|---|
| GAPDH F | SEQ ID NO 89 | ATCACCATCTTCCAGGAGCGA |
| GAPDH R | SEQ ID NO 90 | TTCTCCATGGTGGTGAAGACG |
| CDKN1A F | SEQ ID NO 91 | GGAAGACCATGTGGACCTGT |
| CDKN1A R | SEQ ID NO 92 | GGATTAGGGCTTCCTCTTGG |
| KLF4 F | SEQ ID NO 578 | TTCCCATCTCAAGGCACACC |
| KLF4 R | SEQ ID NO 579 | GCGAATTTCCATCCACAGCC |
| NKX3-1 F | SEQ ID NO 580 | AGGCTTCCCCAAACCCCTAA |
| NKX3-1 R | SEQ ID NO 581 | TCCGTGAGCTTGAGGTTCTT |
| VEGF F | SEQ ID NO 582 | CTTTCTGCTGTCTTGGGTGC |
| VEGF R | SEQ ID NO 583 | TTCGTGATGATTCTGCCCTCC |

Assay of Cell Proliferation

Cells were plated into a 96-well plate with 2-4×10³ cells/well, cultured overnight, and transfected with oligonucleotide duplexes. Three days after transfection, the CCK-8 reagent (Dojindo; Rockville, Md.) was used to assess cell proliferation according to its manual. Briefly, 10 μL of CCK8 reagent was added into each well on the plate which was then incubated at 37° C. for 1 hour. Absorbance for each well on the plate was measured at 450 nm by a microplate reader.

QuantiGene 2.0 Assay

Cells were plated into a 96-well plate and transfected with oligonucleotide duplexes. 72 hours after transfection, the mRNA levels of target genes were quantitatively assayed with a QuantiGene 2.0 kit (AffyMetrix; Santa Clara, Calif.). QuantiGene 2.0 assay is based on hybridization technology, wherein mRNA levels were directly quantified with gene-specific probes. The experimental procedure is briefly described as follows: a lysis solution was added to lyse the transfected cells, and the cell lysates were added into a capture well plate coated with probe for CDKN1A (p21) and HPRT1 which serves as a housekeeping gene for hybridizing overnight at 55° C. In order to enhance the hybridization signal, hybridizations with 2.0 PreAMP Probe, 2.0 AMP Probe, and 2.0 Label Probe were conducted sequentially in 100 μL of a corresponding buffer solution (provided by Quantigene 2.0 kit). All the hybridizations were conducted with shaking at 50-55° C. for 1 hour. After the last wash step, 2.0 Substrate was added to the solution and incubated at room temperature for 5 minutes. Optical signals were detected with an Infinite 200 PRO plate reader (Tecan, Switzerland).

Statistical Analysis

Results were represented as mean±standard deviation. One-way analysis of variance was carried out with GraphPad Prism software (GraphPad Software), and then a Tukey's t test was conducted for pairwise comparisons. A statistical significance was set as *p<0.05, **p<0.01 and ***p<0.001.

### EXAMPLES

The present invention is further illustrated by the following examples. These examples are provided merely for illustration purposes and shall not be interpreted to limit the scope or content of the present invention in any way.

For the convenience of description, the structure of small activating saRNAs (saRNA for short hereinafter) are described by the following formulas.

Description for a Single-Stranded Oligonucleotide:

[5OHₙ]+Nₙ+[3OHₙ] (formula 1)

5OHₙ and 3OHₙ represents an overhang at the 5′ and 3′ terminus respectively, which can be a natural nucleotide overhang (natural overhang, NO), a deoxythymine nucleotide (T) overhang or a uracil nucleotide (U) overhang, wherein n represents the number of overhanging nucleotides; and Nₙ represents a region capable of forming a double-stranded structure with another oligonucleotide strand, wherein N can be S (sense strand) or AS (antisense strand), and n represents the number of nucleotides (excluding the overhang) for forming a double-stranded structure with another complementary strand. For example, 5NO₁+AS₁₉ 3 T₂ represents an antisense strand that can form a double-stranded region of 19 nucleotides in length with another complementary strand, wherein there is an overhang having one natural nucleotide at the 5′ terminus and an overhang having two deoxythymine nucleotides at the 3′ terminus, and the total length is 22 nucleotides (i.e., 1+19+2). Square brackets represent an option.

Description for a Double-Stranded Oligonucleotide:

[B₅]+[B₃]+[MM_{(N′nZ: X,Y)}]+[OH_{N5′}] (formula 2)

B₅ and B₃ represent a 5′ and 3′ blunt end respectively relative to the duplex or to the 5′ terminus of the sense strand; MM represents mismatches between the two strands; Further, in the expression of (N′n Z: X, Y), n represents the position of a mispaired nucleotide in the duplex counted either from N′ terminus of the duplex, wherein N′ can be 5′ or 3′ terminus, X represents an unmutated nucleotide identical or complementary with a target sequence, Y represents a mutated nucleotide, Z represents the strand where the mutated nucleotide is located and can be S (for sense) or AS (for antisense). For example, “3′ 1S:A, C” means that “A” (adenine nucleotide) of the sense strand at the first position counted from the 3′ terminus of the duplex is mutated into “C” (cytosine nucleotide). OH_{N5′} represents an unconventional 5′ overhang, and N represents S (sense) or AS (antisense) strand. For example, OH_{AS5′} means that a 5′ overhang exists in the AS strand. A duplex containing symmetric 3′ overhangs is regarded as a conventional structure and will not be specifically described with formula. Square brackets represent an option.

### Example 1: Screening of Functional saRNAs Targeting the Promoter Region of p21 Gene

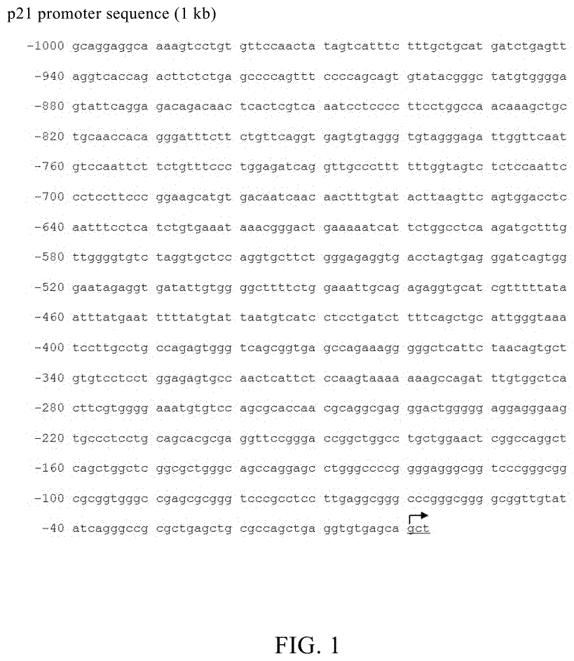

The coding strand (FIG. 1) for the 1 kb promoter sequence of p21 (SEQ ID NO:601) was retrieved from the UCSC Genome database to screen for functional saRNAs capable of activating p21 gene expression. A total of 982 target sequences were obtained by selecting a target with a size of 19 bp starting from the −1 kb position upstream of the TSS and moving toward the TSS one base pair (bp) at a time. The target sequences were filtered to remove those that have a GC content higher than 65% or lower than 35% and those contain 5 or more consecutive nucleotides. After filtration of the target sequences, 439 target sequences remained and were used as candidates for screening. A RNA sequence of 19 nucleotides in size with 100% sequence homology with each of the candidate target sequence was synthesized chemically, and a dTdT dinucleotide was added to its 3′ terminus to obtain a sense strand with a structural formula as S₁₉+[3T₂]. Meanwhile, a RNA sequence of 19 nucleotides in size with 100% complementarity with the same target sequence was synthesized, and a dTdT dinucleotide was added to its 3′ terminus to obtain an antisense strand with a structural formula as AS₁₉+[3T₂]. The sense and the antisense RNA strands were mixed in the same mole number and annealed to obtain a double-stranded saRNA.

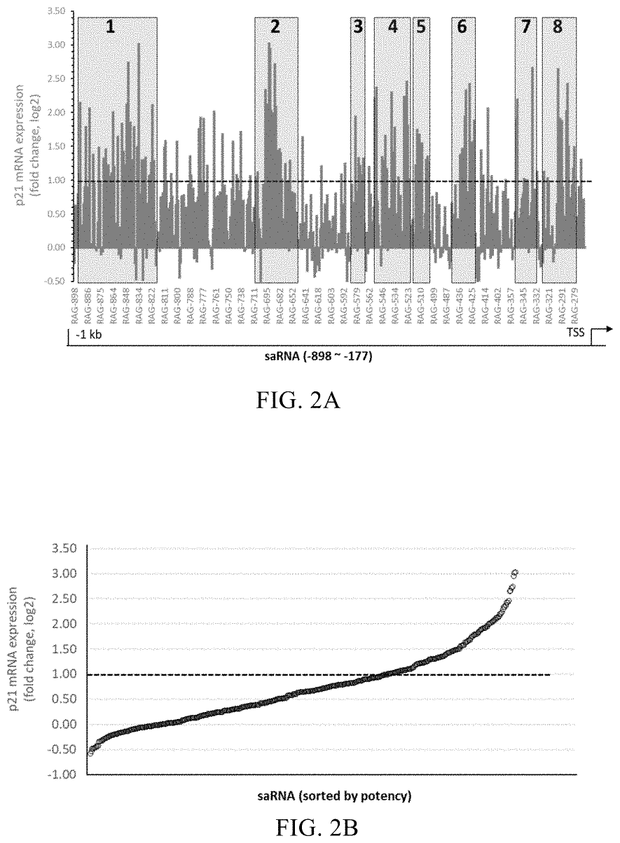

The aforementioned double-stranded saRNA was transfected into Ku-7-luc2-GFP cells at the final concentration of 10 nM, and 72 hours after transfection, the mRNA level of p21 was detected with QuantiGene 2.0 kit. The fold change of p21 mRNA level induced by each saRNA relative to the blank control was calculated and plotted in FIG. 2. In this study, the fold changes of p21mRNA induced by all saRNAs ranged from 0.66 (suppression) to 8.12 (induction) (FIG. 2B). 361 (82.2%) saRNAs induced a 1.01-fold to 8.12-fold change of p21 mRNA expression, 74 (16.9%) saRNAs exhibited a suppressing effect (0.99-fold to 0.66-fold change), and 4 (0.9%) saRNAs did not change p21 mRNA level (1.0-fold).

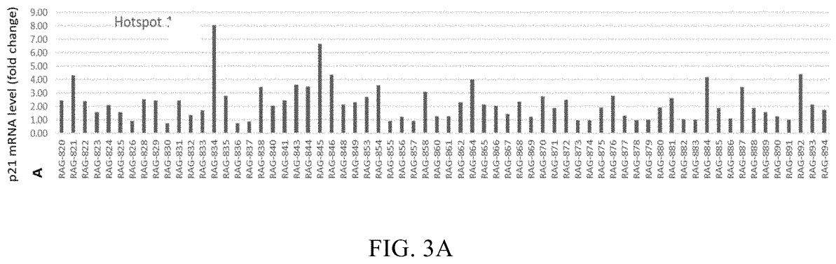

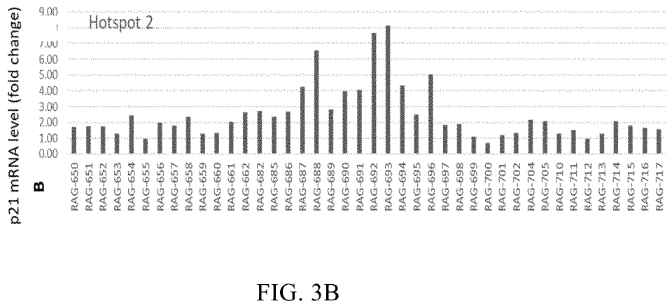

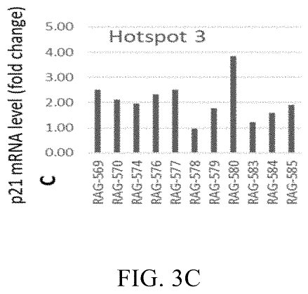

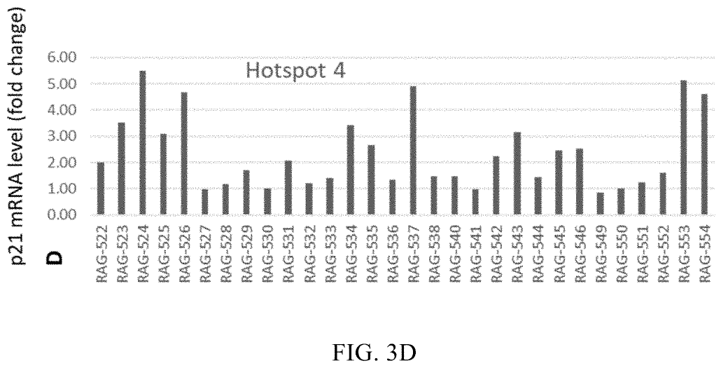

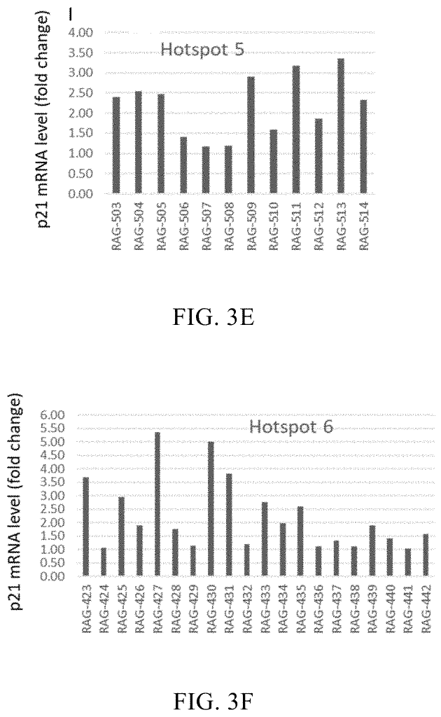

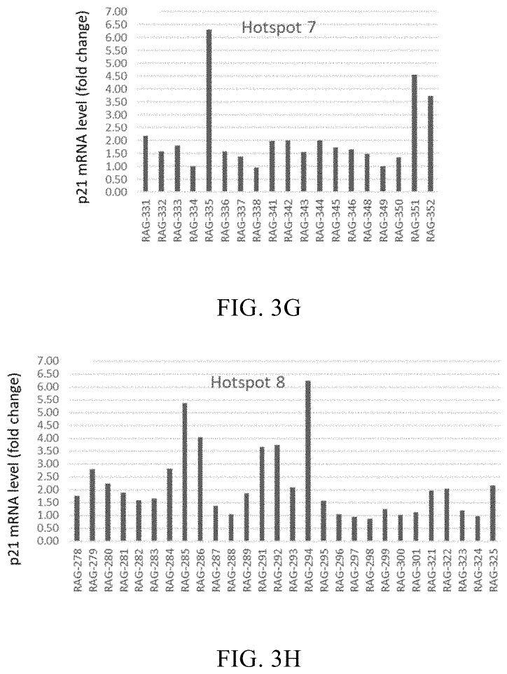

Among the 439 screened saRNAs, 132 saRNAs (30.1%) could induce at least a 2-fold change, and 229 saRNAs (52.4%) could induce at least a 1.5-fold change in p21 mRNA level. The targets for these functional saRNAs were scattered in the entire p21 promoter region. However, 8 discrete regions showed enrichment for saRNA targets and these regions are considered as saRNA “hotspots”. The hotspot is defined as a region containing targets at least for 10 corresponding saRNAs, wherein at least 60% of them can induce a 1.5-fold or more change in the mRNA expression of p21 (FIG. 2A and FIG. 3). The target sequences for hotspots 1 to hotspot 8 and their corresponding saRNA sequences are listed in Table 3 and Table 4 respectively.

TABLE 3
Target sequences of hotspot regions of p21 promoter

| | | | | Length |
|---|---|---|---|---|
| Hotspot | Sequence No. | DNA Sequence (5′-3′) | Range | (bp) |
| Hotspot 1 | SEQ ID NO: 93 | ggctatgtggggagtattcaggagacagacaactcact | −893~−801 | 92 |
| | | cgtcaaatcctccccttcctggccaacaaagctgctgca | | |
| | | accacagggatttct | | |
| Hotspot 2 | SEQ ID NO: 94 | ggtagtctctccaattccctccttcccggaagcatgtgac | −717~−632 | 86 |
| | | aatcaacaactttgtatacttaagttcagtggacctcaattt | | |
| | | cctc | | |
| Hotspot 3 | SEQ ID NO: 95 | ctttgttggggtgtctaggtgctccaggtgcttct | −585~−551 | 35 |
| Hotspot 4 | SEQ ID NO: 96 | ttctgggagaggtgacctagtgagggatcagtgggaat | −554~−504 | 51 |
| | | agaggtgatattg | | |
| Hotspot 5 | SEQ ID NO: 97 | aggtgatattgtggggcttttctggaaatt | −514~−485 | 30 |
| Hotspot 6 | SEQ ID NO: 98 | attaatgtcatcctcctgatcttttcagctgcattggg | −442~−405 | 38 |
| Hotspot 7 | SEQ ID NO: 99 | tctaacagtgctgtgtcctcctggagagtgccaactcatt | −352~−313 | 40 |
| Hotspot 8 | SEQ ID NO: 100 | gtgccaactcattctccaagtaaaaaaagccagatttgtg | −325~−260 | 66 |
| | | gctcacttcgtggggaaatgtgtcca | | |

TABLE 4
functional saRNAs located in hotspot regions of p21 promoter

| Title | Sequence No. | Sequence (5′-3′) | Length |
|---|---|---|---|
| Hotspot 8 | | | |
| RAG-278 | SEQ ID NO: 101 | UCGUGGGGAAAUGUGUCCA[dT][dT] | 21 nt |
| | SEQ ID NO: 102 | UGGACACAUUUCCCCACGA[dT][dT] | 21 nt |
| RAG-279 | SEQ ID NO: 103 | UUCGUGGGGAAAUGUGUCC[dT][dT] | 21 nt |
| | SEQ ID NO: 104 | GGACACAUUUCCCCACGAA[dT][dT] | 21 nt |
| RAG-280 | SEQ ID NO: 105 | CUUCGUGGGGAAAUGUGUC[dT][dT] | 21 nt |
| | SEQ ID NO: 106 | GACACAUUUCCCCACGAAG[dT][dT] | 21 nt |
| RAG-281 | SEQ ID NO: 107 | ACUUCGUGGGGAAAUGUGU[dT][dT] | 21 nt |
| | SEQ ID NO: 108 | ACACAUUUCCCCACGAAGU[dT][dT] | 21 nt |
| RAG-282 | SEQ ID NO: 109 | CACUUCGUGGGGAAAUGUG[dT][dT] | 21 nt |
| | SEQ ID NO: 110 | CACAUUUCCCCACGAAGUG[dT][dT] | 21 nt |
| RAG-283 | SEQ ID NO: 111 | UCACUUCGUGGGGAAAUGU[dT][dT] | 21 nt |
| | SEQ ID NO: 112 | ACAUUUCCCCACGAAGUGA[dT][dT] | 21 nt |
| RAG-284 | SEQ ID NO: 113 | CUCACUUCGUGGGGAAAUG[dT][dT] | 21 nt |
| | SEQ ID NO: 114 | CAUUUCCCCACGAAGUGAG[dT][dT] | 21 nt |
| RAG-285 | SEQ ID NO: 115 | GCUCACUUCGUGGGGAAAU[dT][dT] | 21 nt |
| | SEQ ID NO: 116 | AUUUCCCCACGAAGUGAGC[dT][dT] | 21 nt |
| RAG-286 | SEQ ID NO: 117 | GGCUCACUUCGUGGGGAAA[dT][dT] | 21 nt |
| | SEQ ID NO: 118 | UUUCCCCACGAAGUGAGCC[dT][dT] | 21 nt |
| RAG-287 | SEQ ID NO: 119 | UGGCUCACUUCGUGGGGAA[dT][dT] | 21 nt |
| | SEQ ID NO: 120 | UUCCCCACGAAGUGAGCCA[dT][dT] | 21 nt |
| RAG-288 | SEQ ID NO: 121 | GUGGCUCACUUCGUGGGGA[dT][dT] | 21 nt |
| | SEQ ID NO: 122 | UCCCCACGAAGUGAGCCAC[dT][dT] | 21 nt |
| RAG-289 | SEQ ID NO: 123 | UGUGGCUCACUUCGUGGGG[dT][dT] | 21 nt |
| | SEQ ID NO: 124 | CCCCACGAAGUGAGCCACA[dT][dT] | 21 nt |
| RAG-291 | SEQ ID NO: 125 | UUUGUGGCUCACUUCGUGG[dT][dT] | 21 nt |
| | SEQ ID NO: 126 | CCACGAAGUGAGCCACAAA[dT][dT] | 21 nt |
| RAG-292 | SEQ ID NO: 127 | AUUUGUGGCUCACUUCGUG[dT][dT] | 21 nt |
| | SEQ ID NO: 128 | CACGAAGUGAGCCACAAAU[dT][dT] | 21 nt |
| RAG-293 | SEQ ID NO: 129 | GAUUUGUGGCUCACUUCGU[dT][dT] | 21 nt |
| | SEQ ID NO: 130 | ACGAAGUGAGCCACAAAUC[dT][dT] | 21 nt |
| RAG-294 | SEQ ID NO: 131 | AGAUUUGUGGCUCACUUCG[dT][dT] | 21 nt |
| | SEQ ID NO: 132 | CGAAGUGAGCCACAAAUCU[dT][dT] | 21 nt |
| RAG-295 | SEQ ID NO: 133 | CAGAUUUGUGGCUCACUUC[dT][dT] | 21 nt |
| | SEQ ID NO: 134 | GAAGUGAGCCACAAAUCUG[dT][dT] | 21 nt |
| RAG-296 | SEQ ID NO: 135 | CCAGAUUUGUGGCUCACUU[dT][dT] | 21 nt |
| | SEQ ID NO: 136 | AAGUGAGCCACAAAUCUGG[dT][dT] | 21 nt |
| RAG-297 | SEQ ID NO: 137 | GCCAGAUUUGUGGCUCACU[dT][dT] | 21 nt |
| | SEQ ID NO: 138 | AGUGAGCCACAAAUCUGGC[dT][dT] | 21 nt |
| RAG-298 | SEQ ID NO: 139 | AGCCAGAUUUGUGGCUCAC[dT][dT] | 21 nt |
| | SEQ ID NO: 140 | GUGAGCCACAAAUCUGGCU[dT][dT] | 21 nt |
| RAG-299 | SEQ ID NO: 141 | AAGCCAGAUUUGUGGCUCA[dT][dT] | 21 nt |
| | SEQ ID NO: 142 | UGAGCCACAAAUCUGGCUU[dT][dT] | 21 nt |
| RAG-300 | SEQ ID NO: 143 | AAAGCCAGAUUUGUGGCUC[dT][dT] | 21 nt |
| | SEQ ID NO: 144 | GAGCCACAAAUCUGGCUUU[dT][dT] | 21 nt |
| RAG-301 | SEQ ID NO: 145 | AAAAGCCAGAUUUGUGGCU[dT][dT] | 21 nt |
| | SEQ ID NO: 146 | AGCCACAAAUCUGGCUUUU[dT][dT] | 21 nt |
| RAG-321 | SEQ ID NO: 147 | CAACUCAUUCUCCAAGUAA[dT][dT] | 21 nt |
| | SEQ ID NO: 148 | UUACUUGGAGAAUGAGUUG[dT][dT] | 21 nt |
| RAG-322 | SEQ ID NO: 149 | CCAACUCAUUCUCCAAGUA[dT][dT] | 21 nt |
| | SEQ ID NO: 150 | UACUUGGAGAAUGAGUUGG[dT][dT] | 21 nt |
| RAG-323 | SEQ ID NO: 151 | GCCAACUCAUUCUCCAAGU[dT][dT] | 21 nt |
| | SEQ ID NO: 152 | ACUUGGAGAAUGAGUUGGC[dT][dT] | 21 nt |
| RAG-324 | SEQ ID NO: 153 | UGCCAACUCAUUCUCCAAG[dT][dT] | 21 nt |
| | SEQ ID NO: 154 | CUUGGAGAAUGAGUUGGCA[dT][dT] | 21 nt |
| RAG-325 | SEQ ID NO: 155 | GUGCCAACUCAUUCUCCAA[dT][dT] | 21 nt |
| | SEQ ID NO: 156 | UUGGAGAAUGAGUUGGCAC[dT][dT] | 21 nt |
| Hotspot 7 | | | |
| RAG-331 | SEQ ID NO: 157 | UGGAGAGUGCCAACUCAUU[dT][dT] | 21 nt |
| | SEQ ID NO: 158 | AAUGAGUUGGCACUCUCCA[dT][dT] | 21 nt |
| RAG-332 | SEQ ID NO: 159 | CUGGAGAGUGCCAACUCAU[dT][dT] | 21 nt |
| | SEQ ID NO: 160 | AUGAGUUGGCACUCUCCAG[dT][dT] | 21 nt |
| RAG-333 | SEQ ID NO: 161 | CCUGGAGAGUGCCAACUCA[dT][dT] | 21 nt |
| | SEQ ID NO: 162 | UGAGUUGGCACUCUCCAGG[dT][dT] | 21 nt |
| RAG-334 | SEQ ID NO: 163 | UCCUGGAGAGUGCCAACUC[dT][dT] | 21 nt |
| | SEQ ID NO: 164 | GAGUUGGCACUCUCCAGGA[dT][dT] | 21 nt |
| RAG-335 | SEQ ID NO: 165 | CUCCUGGAGAGUGCCAACU[dT][dT] | 21 nt |
| | SEQ ID NO: 166 | AGUUGGCACUCUCCAGGAG[dT][dT] | 21 nt |
| RAG-336 | SEQ ID NO: 167 | CCUCCUGGAGAGUGCCAAC[dT][dT] | 21 nt |
| | SEQ ID NO: 168 | GUUGGCACUCUCCAGGAGG[dT][dT] | 21 nt |
| RAG-337 | SEQ ID NO: 169 | UCCUCCUGGAGAGUGCCAA[dT][dT] | 21 nt |
| | SEQ ID NO: 170 | UUGGCACUCUCCAGGAGGA[dT][dT] | 21 nt |
| RAG-338 | SEQ ID NO: 171 | GUCCUCCUGGAGAGUGCCA[dT][dT] | 21 nt |
| | SEQ ID NO: 172 | UGGCACUCUCCAGGAGGAC[dT][dT] | 21 nt |
| RAG-341 | SEQ ID NO: 173 | UGUGUCCUCCUGGAGAGUG[dT][dT] | 21 nt |
| | SEQ ID NO: 174 | CACUCUCCAGGAGGACACA[dT][dT] | 21 nt |
| RAG-342 | SEQ ID NO: 175 | CUGUGUCCUCCUGGAGAGU[dT][dT] | 21 nt |
| | SEQ ID NO: 176 | ACUCUCCAGGAGGACACAG[dT][dT] | 21 nt |
| RAG-343 | SEQ ID NO: 177 | GCUGUGUCCUCCUGGAGAG[dT][dT] | 21 nt |
| | SEQ ID NO: 178 | CUCUCCAGGAGGACACAGC[dT][dT] | 21 nt |
| RAG-344 | SEQ ID NO: 179 | UGCUGUGUCCUCCUGGAGA[dT][dT] | 21 nt |
| | SEQ ID NO: 180 | UCUCCAGGAGGACACAGCA[dT][dT] | 21 nt |
| RAG-345 | SEQ ID NO: 181 | GUGCUGUGUCCUCCUGGAG[dT][dT] | 21 nt |
| | SEQ ID NO: 182 | CUCCAGGAGGACACAGCAC[dT][dT] | 21 nt |
| RAG-346 | SEQ ID NO: 183 | AGUGCUGUGUCCUCCUGGA[dT][dT] | 21 nt |
| | SEQ ID NO: 184 | UCCAGGAGGACACAGCACU[dT][dT] | 21 nt |
| RAG-348 | SEQ ID NO: 185 | ACAGUGCUGUGUCCUCCUG[dT][dT] | 21 nt |
| | SEQ ID NO: 186 | CAGGAGGACACAGCACUGU[dT][dT] | 21 nt |
| RAG-349 | SEQ ID NO: 187 | AACAGUGCUGUGUCCUCCU[dT][dT] | 21 nt |
| | SEQ ID NO: 188 | AGGAGGACACAGCACUGUU[dT][dT] | 21 nt |
| RAG-350 | SEQ ID NO: 189 | UAACAGUGCUGUGUCCUCC[dT][dT] | 21 nt |
| | SEQ ID NO: 190 | GGAGGACACAGCACUGUUA[dT][dT] | 21 nt |
| RAG-351 | SEQ ID NO: 191 | CUAACAGUGCUGUGUCCUC[dT][dT] | 21 nt |
| | SEQ ID NO: 192 | GAGGACACAGCACUGUUAG[dT][dT] | 21 nt |
| RAG-352 | SEQ ID NO: 193 | UCUAACAGUGCUGUGUCCU[dT][dT] | 21 nt |
| | SEQ ID NO: 194 | AGGACACAGCACUGUUAGA[dT][dT] | 21 nt |
| Hotspot 6 | | | |
| RAG-423 | SEQ ID NO: 195 | UCUUUUCAGCUGCAUUGGG[dT][dT] | 21 nt |
| | SEQ ID NO: 196 | CCCAAUGCAGCUGAAAAGA[dT][dT] | 21 nt |
| RAG-424 | SEQ ID NO: 197 | AUCUUUUCAGCUGCAUUGG[dT][dT] | 21 nt |
| | SEQ ID NO: 198 | CCAAUGCAGCUGAAAAGAU[dT][dT] | 21 nt |
| RAG-425 | SEQ ID NO: 199 | GAUCUUUUCAGCUGCAUUG[dT][dT] | 21 nt |
| | SEQ ID NO: 200 | CAAUGCAGCUGAAAAGAUC[dT][dT] | 21 nt |
| RAG-426 | SEQ ID NO: 201 | UGAUCUUUUCAGCUGCAUU[dT][dT] | 21 nt |
| | SEQ ID NO: 202 | AAUGCAGCUGAAAAGAUCA[dT][dT] | 21 nt |
| RAG-427 | SEQ ID NO: 203 | CUGAUCUUUUCAGCUGCAU[dT][dT] | 21 nt |
| | SEQ ID NO: 204 | AUGCAGCUGAAAAGAUCAG[dT][dT] | 21 nt |
| RAG-428 | SEQ ID NO: 205 | CCUGAUCUUUUCAGCUGCA[dT][dT] | 21 nt |
| | SEQ ID NO: 206 | UGCAGCUGAAAAGAUCAGG[dT][dT] | 21 nt |
| RAG-429 | SEQ ID NO: 207 | UCCUGAUCUUUUCAGCUGC[dT][dT] | 21 nt |
| | SEQ ID NO: 208 | GCAGCUGAAAAGAUCAGGA[dT][dT] | 21 nt |
| RAG-430 | SEQ ID NO: 209 | CUCCUGAUCUUUUCAGCUG[dT][dT] | 21 nt |
| | SEQ ID NO: 210 | CAGCUGAAAAGAUCAGGAG[dT][dT] | 21 nt |
| RAG-431 | SEQ ID NO: 211 | CCUCCUGAUCUUUUCAGCU[dT][dT] | 21 nt |
| | SEQ ID NO: 212 | AGCUGAAAAGAUCAGGAGG[dT][dT] | 21 nt |
| RAG-432 | SEQ ID NO: 213 | UCCUCCUGAUCUUUUCAGC[dT][dT] | 21 nt |
| | SEQ ID NO: 214 | GCUGAAAAGAUCAGGAGGA[dT][dT] | 21 nt |
| RAG-433 | SEQ ID NO: 215 | AUCCUCCUGAUCUUUUCAG[dT][dT] | 21 nt |
| | SEQ ID NO: 216 | CUGAAAAGAUCAGGAGGAU[dT][dT] | 21 nt |
| RAG-434 | SEQ ID NO: 217 | CAUCCUCCUGAUCUUUUCA[dT][dT] | 21 nt |
| | SEQ ID NO: 218 | UGAAAAGAUCAGGAGGAUG[dT][dT] | 21 nt |
| RAG-435 | SEQ ID NO: 219 | UCAUCCUCCUGAUCUUUUC[dT][dT] | 21 nt |
| | SEQ ID NO: 220 | GAAAAGAUCAGGAGGAUGA[dT][dT] | 21 nt |
| RAG-436 | SEQ ID NO: 221 | GUCAUCCUCCUGAUCUUUU[dT][dT] | 21 nt |
| | SEQ ID NO: 222 | AAAAGAUCAGGAGGAUGAC[dT][dT] | 21 nt |
| RAG-437 | SEQ ID NO: 223 | UGUCAUCCUCCUGAUCUUU[dT][dT] | 21 nt |
| | SEQ ID NO: 224 | AAAGAUCAGGAGGAUGACA[dT][dT] | 21 nt |
| RAG-438 | SEQ ID NO: 225 | AUGUCAUCCUCCUGAUCUU[dT][dT] | 21 nt |
| | SEQ ID NO: 226 | AAGAUCAGGAGGAUGACAU[dT][dT] | 21 nt |
| RAG-439 | SEQ ID NO: 227 | AAUGUCAUCCUCCUGAUCU[dT][dT] | 21 nt |
| | SEQ ID NO: 228 | AGAUCAGGAGGAUGACAUU[dT][dT] | 21 nt |
| RAG-440 | SEQ ID NO: 229 | UAAUGUCAUCCUCCUGAUC[dT][dT] | 21 nt |
| | SEQ ID NO: 230 | GAUCAGGAGGAUGACAUUA[dT][dT] | 21 nt |
| RAG-441 | SEQ ID NO: 231 | UUAAUGUCAUCCUCCUGAU[dT][dT] | 21 nt |
| | SEQ ID NO: 232 | AUCAGGAGGAUGACAUUAA[dT][dT] | 21 nt |
| RAG-442 | SEQ ID NO: 233 | AUUAAUGUCAUCCUCCUGA[dT][dT] | 21 nt |
| | SEQ ID NO: 234 | UCAGGAGGAUGACAUUAAU[dT][dT] | 21 nt |
| Hotspot 5 | | | |
| RAG-503 | SEQ ID NO: 235 | UGGGGCUUUUCUGGAAAUU[dT][dT] | 21 nt |
| | SEQ ID NO: 236 | AAUUUCCAGAAAAGCCCCA[dT][dT] | 21 nt |
| RAG-504 | SEQ ID NO: 237 | GUGGGGCUUUUCUGGAAAU[dT][dT] | 21 nt |
| | SEQ ID NO: 238 | AUUUCCAGAAAAGCCCCAC[dT][dT] | 21 nt |
| RAG-505 | SEQ ID NO: 239 | UGUGGGGCUUUUCUGGAAA[dT][dT] | 21 nt |
| | SEQ ID NO: 240 | UUUCCAGAAAAGCCCCACA[dT][dT] | 21 nt |
| RAG-506 | SEQ ID NO: 241 | UUGUGGGGCUUUUCUGGAA[dT][dT] | 21 nt |
| | SEQ ID NO: 242 | UUCCAGAAAAGCCCCACAA[dT][dT] | 21 nt |
| RAG-507 | SEQ ID NO: 243 | AUUGUGGGGCUUUUCUGGA[dT][dT] | 21 nt |
| | SEQ ID NO: 244 | UCCAGAAAAGCCCCACAAU[dT][dT] | 21 nt |
| RAG-508 | SEQ ID NO: 245 | UAUUGUGGGGCUUUUCUGG[dT][dT] | 21 nt |
| | SEQ ID NO: 246 | CCAGAAAAGCCCCACAAUA[dT][dT] | 21 nt |
| RAG-509 | SEQ ID NO: 247 | AUAUUGUGGGGCUUUUCUG[dT][dT] | 21 nt |
| | SEQ ID NO: 248 | CAGAAAAGCCCCACAAUAU[dT][dT] | 21 nt |
| RAG-510 | SEQ ID NO: 249 | GAUAUUGUGGGGCUUUUCU[dT][dT] | 21 nt |
| | SEQ ID NO: 250 | AGAAAAGCCCCACAAUAUC[dT][dT] | 21 nt |
| RAG-511 | SEQ ID NO: 251 | UGAUAUUGUGGGGCUUUUC[dT][dT] | 21 nt |
| | SEQ ID NO: 252 | GAAAAGCCCCACAAUAUCA[dT][dT] | 21 nt |
| RAG-512 | SEQ ID NO: 253 | GUGAUAUUGUGGGGCUUUU[dT][dT] | 21 nt |
| | SEQ ID NO: 254 | AAAAGCCCCACAAUAUCAC[dT][dT] | 21 nt |
| RAG-513 | SEQ ID NO: 255 | GGUGAUAUUGUGGGGCUUU[dT][dT] | 21 nt |
| | SEQ ID NO: 256 | AAAGCCCCACAAUAUCACC[dT][dT] | 21 nt |
| RAG-514 | SEQ ID NO: 257 | AGGUGAUAUUGUGGGGCUU[dT][dT] | 21 nt |
| | SEQ ID NO: 258 | AAGCCCCACAAUAUCACCU[dT][dT] | 21 nt |
| Hotspot 4 | | | |
| RAG-522 | SEQ ID NO: 259 | GGGAAUAGAGGUGAUAUUG[dT][dT] | 21 nt |
| | SEQ ID NO: 260 | CAAUAUCACCUCUAUUCCC[dT][dT] | 21 nt |
| RAG-523 | SEQ ID NO: 261 | UGGGAAUAGAGGUGAUAUU[dT][dT] | 21 nt |
| | SEQ ID NO: 262 | AAUAUCACCUCUAUUCCCA[dT][dT] | 21 nt |
| RAG-524 | SEQ ID NO: 263 | GUGGGAAUAGAGGUGAUAU[dT][dT] | 21 nt |
| | SEQ ID NO: 264 | AUAUCACCUCUAUUCCCAC[dT][dT] | 21 nt |
| RAG-525 | SEQ ID NO: 265 | AGUGGGAAUAGAGGUGAUA[dT][dT] | 21 nt |
| | SEQ ID NO: 266 | UAUCACCUCUAUUCCCACU[dT][dT] | 21 nt |
| RAG-526 | SEQ ID NO: 267 | CAGUGGGAAUAGAGGUGAU[dT][dT] | 21 nt |
| | SEQ ID NO: 268 | AUCACCUCUAUUCCCACUG[dT][dT] | 21 nt |
| RAG-527 | SEQ ID NO: 269 | UCAGUGGGAAUAGAGGUGA[dT][dT] | 21 nt |
| | SEQ ID NO: 270 | UCACCUCUAUUCCCACUGA[dT][dT] | 21 nt |
| RAG-528 | SEQ ID NO: 271 | AUCAGUGGGAAUAGAGGUG[dT][dT] | 21 nt |
| | SEQ ID NO: 272 | CACCUCUAUUCCCACUGAU[dT][dT] | 21 nt |
| RAG-529 | SEQ ID NO: 273 | GAUCAGUGGGAAUAGAGGU[dT][dT] | 21 nt |
| | SEQ ID NO: 274 | ACCUCUAUUCCCACUGAUC[dT][dT] | 21 nt |
| RAG-530 | SEQ ID NO: 275 | GGAUCAGUGGGAAUAGAGG[dT][dT] | 21 nt |
| | SEQ ID NO: 276 | CCUCUAUUCCCACUGAUCC[dT][dT] | 21 nt |
| RAG-531 | SEQ ID NO: 277 | GGGAUCAGUGGGAAUAGAG[dT][dT] | 21 nt |
| | SEQ ID NO: 278 | CUCUAUUCCCACUGAUCCC[dT][dT] | 21 nt |
| RAG-532 | SEQ ID NO: 279 | AGGGAUCAGUGGGAAUAGA[dT][dT] | 21 nt |
| | SEQ ID NO: 280 | UCUAUUCCCACUGAUCCCU[dT][dT] | 21 nt |
| RAG-533 | SEQ ID NO: 281 | GAGGGAUCAGUGGGAAUAG[dT][dT] | 21 nt |
| | SEQ ID NO: 282 | CUAUUCCCACUGAUCCCUC[dT][dT] | 21 nt |
| RAG-534 | SEQ ID NO: 283 | UGAGGGAUCAGUGGGAAUA[dT][dT] | 21 nt |
| | SEQ ID NO: 284 | UAUUCCCACUGAUCCCUCA[dT][dT] | 21 nt |
| RAG-535 | SEQ ID NO: 285 | GUGAGGGAUCAGUGGGAAU[dT][dT] | 21 nt |
| | SEQ ID NO: 286 | AUUCCCACUGAUCCCUCAC[dT][dT] | 21 nt |
| RAG-536 | SEQ ID NO: 287 | AGUGAGGGAUCAGUGGGAA[dT][dT] | 21 nt |
| | SEQ ID NO: 288 | UUCCCACUGAUCCCUCACU[dT][dT] | 21 nt |
| RAG-537 | SEQ ID NO: 289 | UAGUGAGGGAUCAGUGGGA[dT][dT] | 21 nt |
| | SEQ ID NO: 290 | UCCCACUGAUCCCUCACUA[dT][dT] | 21 nt |
| RAG-538 | SEQ ID NO: 291 | CUAGUGAGGGAUCAGUGGG[dT][dT] | 21 nt |
| | SEQ ID NO: 292 | CCCACUGAUCCCUCACUAG[dT][dT] | 21 nt |
| RAG-540 | SEQ ID NO: 293 | ACCUAGUGAGGGAUCAGUG[dT][dT] | 21 nt |
| | SEQ ID NO: 294 | CACUGAUCCCUCACUAGGU[dT][dT] | 21 nt |
| RAG-541 | SEQ ID NO: 295 | GACCUAGUGAGGGAUCAGU[dT][dT] | 21 nt |
| | SEQ ID NO: 296 | ACUGAUCCCUCACUAGGUC[dT][dT] | 21 nt |
| RAG-542 | SEQ ID NO: 297 | UGACCUAGUGAGGGAUCAG[dT][dT] | 21 nt |
| | SEQ ID NO: 298 | CUGAUCCCUCACUAGGUCA[dT][dT] | 21 nt |
| RAG-543 | SEQ ID NO: 299 | GUGACCUAGUGAGGGAUCA[dT][dT] | 21 nt |
| | SEQ ID NO: 300 | UGAUCCCUCACUAGGUCAC[dT][dT] | 21 nt |
| RAG-544 | SEQ ID NO: 301 | GGUGACCUAGUGAGGGAUC[dT][dT] | 21 nt |
| | SEQ ID NO: 302 | GAUCCCUCACUAGGUCACC[dT][dT] | 21 nt |
| RAG-545 | SEQ ID NO: 303 | AGGUGACCUAGUGAGGGAU[dT][dT] | 21 nt |
| | SEQ ID NO: 304 | AUCCCUCACUAGGUCACCU[dT][dT] | 21 nt |
| RAG-546 | SEQ ID NO: 305 | GAGGUGACCUAGUGAGGGA[dT][dT] | 21 nt |
| | SEQ ID NO: 306 | UCCCUCACUAGGUCACCUC[dT][dT] | 21 nt |
| RAG-549 | SEQ ID NO: 307 | GGAGAGGUGACCUAGUGAG[dT][dT] | 21 nt |
| | SEQ ID NO: 308 | CUCACUAGGUCACCUCUCC[dT][dT] | 21 nt |
| RAG-550 | SEQ ID NO: 309 | GGGAGAGGUGACCUAGUGA[dT][dT] | 21 nt |
| | SEQ ID NO: 310 | UCACUAGGUCACCUCUCCC[dT][dT] | 21 nt |
| RAG-551 | SEQ ID NO: 311 | UGGGAGAGGUGACCUAGUG[dT][dT] | 21 nt |
| | SEQ ID NO: 312 | CACUAGGUCACCUCUCCCA[dT][dT] | 21 nt |
| RAG-552 | SEQ ID NO: 313 | CUGGGAGAGGUGACCUAGU[dT][dT] | 21 nt |
| | SEQ ID NO: 314 | ACUAGGUCACCUCUCCCAG[dT][dT] | 21 nt |
| RAG-553 | SEQ ID NO: 315 | UCUGGGAGAGGUGACCUAG[dT][dT] | 21 nt |
| | SEQ ID NO: 316 | CUAGGUCACCUCUCCCAGA[dT][dT] | 21 nt |
| RAG-554 | SEQ ID NO: 317 | UUCUGGGAGAGGUGACCUA[dT][dT] | 21 nt |
| | SEQ ID NO: 318 | UAGGUCACCUCUCCCAGAA[dT][dT] | 21 nt |
| Hotspot 3 | | | |
| RAG-569 | SEQ ID NO: 319 | AGGUGCUCCAGGUGCUUCU[dT][dT] | 21 nt |
| | SEQ ID NO: 320 | AGAAGCACCUGGAGCACCU[dT][dT] | 21 nt |
| RAG-570 | SEQ ID NO: 321 | UAGGUGCUCCAGGUGCUUC[dT][dT] | 21 nt |
| | SEQ ID NO: 322 | GAAGCACCUGGAGCACCUA[dT][dT] | 21 nt |
| RAG-574 | SEQ ID NO: 323 | UGUCUAGGUGCUCCAGGUG[dT][dT] | 21 nt |
| | SEQ ID NO: 324 | CACCUGGAGCACCUAGACA[dT][dT] | 21 nt |
| RAG-576 | SEQ ID NO: 325 | GGUGUCUAGGUGCUCCAGG[dT][dT] | 21 nt |
| | SEQ ID NO: 326 | CCUGGAGCACCUAGACACC[dT][dT] | 21 nt |
| RAG-577 | SEQ ID NO: 327 | GGGUGUCUAGGUGCUCCAG[dT][dT] | 21 nt |
| | SEQ ID NO: 328 | CUGGAGCACCUAGACACCC[dT][dT] | 21 nt |
| RAG-578 | SEQ ID NO: 329 | GGGGUGUCUAGGUGCUCCA[dT][dT] | 21 nt |
| | SEQ ID NO: 330 | UGGAGCACCUAGACACCCC[dT][dT] | 21 nt |
| RAG-579 | SEQ ID NO: 331 | UGGGGUGUCUAGGUGCUCC[dT][dT] | 21 nt |
| | SEQ ID NO: 332 | GGAGCACCUAGACACCCCA[dT][dT] | 21 nt |
| RAG-580 | SEQ ID NO: 333 | UUGGGGUGUCUAGGUGCUC[dT][dT] | 21 nt |
| | SEQ ID NO: 334 | GAGCACCUAGACACCCCAA[dT][dT] | 21 nt |
| RAG-583 | SEQ ID NO: 335 | UUGUUGGGGUGUCUAGGUG[dT][dT] | 21 nt |
| | SEQ ID NO: 336 | CACCUAGACACCCCAACAA[dT][dT] | 21 nt |
| RAG-584 | SEQ ID NO: 337 | UUUGUUGGGGUGUCUAGGU[dT][dT] | 21 nt |
| | SEQ ID NO: 338 | ACCUAGACACCCCAACAAA[dT][dT] | 21 nt |
| RAG-585 | SEQ ID NO: 339 | CUUUGUUGGGGUGUCUAGG[dT][dT] | 21 nt |
| | SEQ ID NO: 340 | CCUAGACACCCCAACAAAG[dT][dT] | 21 nt |
| Hotspot 2 | | | |
| RAG-650 | SEQ ID NO: 341 | AGUGGACCUCAAUUUCCUC[dT][dT] | 21 nt |
| | SEQ ID NO: 342 | GAGGAAAUUGAGGUCCACU[dT][dT] | 21 nt |
| RAG-651 | SEQ ID NO: 343 | CAGUGGACCUCAAUUUCCU[dT][dT] | 21 nt |
| | SEQ ID NO: 344 | AGGAAAUUGAGGUCCACUG[dT][dT] | 21 nt |
| RAG-652 | SEQ ID NO: 345 | UCAGUGGACCUCAAUUUCC[dT][dT] | 21 nt |
| | SEQ ID NO: 346 | GGAAAUUGAGGUCCACUGA[dT][dT] | 21 nt |
| RAG-653 | SEQ ID NO: 347 | UUCAGUGGACCUCAAUUUC[dT][dT] | 21 nt |
| | SEQ ID NO: 348 | GAAAUUGAGGUCCACUGAA[dT][dT] | 21 nt |
| RAG-654 | SEQ ID NO: 349 | GUUCAGUGGACCUCAAUUU[dT][dT] | 21 nt |
| | SEQ ID NO: 350 | AAAUUGAGGUCCACUGAAC[dT][dT] | 21 nt |
| RAG-655 | SEQ ID NO: 351 | AGUUCAGUGGACCUCAAUU[dT][dT] | 21 nt |
| | SEQ ID NO: 352 | AAUUGAGGUCCACUGAACU[dT][dT] | 21 nt |
| RAG-656 | SEQ ID NO: 353 | AAGUUCAGUGGACCUCAAU[dT][dT] | 21 nt |
| | SEQ ID NO: 354 | AUUGAGGUCCACUGAACUU[dT][dT] | 21 nt |
| RAG-657 | SEQ ID NO: 355 | UAAGUUCAGUGGACCUCAA[dT][dT] | 21 nt |
| | SEQ ID NO: 356 | UUGAGGUCCACUGAACUUA[dT][dT] | 21 nt |
| RAG-658 | SEQ ID NO: 357 | UUAAGUUCAGUGGACCUCA[dT][dT] | 21 nt |
| | SEQ ID NO: 358 | UGAGGUCCACUGAACUUAA[dT][dT] | 21 nt |
| RAG-659 | SEQ ID NO: 359 | CUUAAGUUCAGUGGACCUC[dT][dT] | 21 nt |
| | SEQ ID NO: 360 | GAGGUCCACUGAACUUAAG[dT][dT] | 21 nt |
| RAG-660 | SEQ ID NO: 361 | ACUUAAGUUCAGUGGACCU[dT][dT] | 21 nt |
| | SEQ ID NO: 362 | AGGUCCACUGAACUUAAGU[dT][dT] | 21 nt |
| RAG-661 | SEQ ID NO: 363 | UACUUAAGUUCAGUGGACC[dT][dT] | 21 nt |
| | SEQ ID NO: 364 | GGUCCACUGAACUUAAGUA[dT][dT] | 21 nt |
| RAG-662 | SEQ ID NO: 365 | AUACUUAAGUUCAGUGGAC[dT][dT] | 21 nt |
| | SEQ ID NO: 366 | GUCCACUGAACUUAAGUAU[dT][dT] | 21 nt |
| RAG-682 | SEQ ID NO: 367 | GUGACAAUCAACAACUUUG[dT][dT] | 21 nt |
| | SEQ ID NO: 368 | CAAAGUUGUUGAUUGUCAC[dT][dT] | 21 nt |
| RAG-685 | SEQ ID NO: 369 | CAUGUGACAAUCAACAACU[dT][dT] | 21 nt |
| | SEQ ID NO: 370 | AGUUGUUGAUUGUCACAUG[dT][dT] | 21 nt |
| RAG-686 | SEQ ID NO: 371 | GCAUGUGACAAUCAACAAC[dT][dT] | 21 nt |
| | SEQ ID NO: 372 | GUUGUUGAUUGUCACAUGC[dT][dT] | 21 nt |
| RAG-687 | SEQ ID NO: 373 | AGCAUGUGACAAUCAACAA[dT][dT] | 21 nt |
| | SEQ ID NO: 374 | UUGUUGAUUGUCACAUGCU[dT][dT] | 21 nt |
| RAG-688 | SEQ ID NO: 375 | AAGCAUGUGACAAUCAACA[dT][dT] | 21 nt |
| | SEQ ID NO: 376 | UGUUGAUUGUCACAUGCUU[dT][dT] | 21 nt |
| RAG-689 | SEQ ID NO: 377 | GAAGCAUGUGACAAUCAAC[dT][dT] | 21 nt |
| | SEQ ID NO: 378 | GUUGAUUGUCACAUGCUUC[dT][dT] | 21 nt |
| RAG-690 | SEQ ID NO: 379 | GGAAGCAUGUGACAAUCAA[dT][dT] | 21 nt |
| | SEQ ID NO: 380 | UUGAUUGUCACAUGCUUCC[dT][dT] | 21 nt |
| RAG-691 | SEQ ID NO: 381 | CGGAAGCAUGUGACAAUCA[dT][dT] | 21 nt |
| | SEQ ID NO: 382 | UGAUUGUCACAUGCUUCCG[dT][dT] | 21 nt |
| RAG-692 | SEQ ID NO: 383 | CCGGAAGCAUGUGACAAUC[dT][dT] | 21 nt |
| | SEQ ID NO: 384 | GAUUGUCACAUGCUUCCGG[dT][dT] | 21 nt |
| RAG-693 | SEQ ID NO: 385 | CCCGGAAGCAUGUGACAAU[dT][dT] | 21 nt |
| | SEQ ID NO: 386 | AUUGUCACAUGCUUCCGGG[dT][dT] | 21 nt |
| RAG-694 | SEQ ID NO: 387 | UCCCGGAAGCAUGUGACAA[dT][dT] | 21 nt |
| | SEQ ID NO: 388 | UUGUCACAUGCUUCCGGGA[dT][dT] | 21 nt |
| RAG-695 | SEQ ID NO: 389 | UUCCCGGAAGCAUGUGACA[dT][dT] | 21 nt |
| | SEQ ID NO: 390 | UGUCACAUGCUUCCGGGAA[dT][dT] | 21 nt |
| RAG-696 | SEQ ID NO: 391 | CUUCCCGGAAGCAUGUGAC[dT][dT] | 21 nt |
| | SEQ ID NO: 392 | GUCACAUGCUUCCGGGAAG[dT][dT] | 21 nt |
| RAG-697 | SEQ ID NO: 393 | CCUUCCCGGAAGCAUGUGA[dT][dT] | 21 nt |
| | SEQ ID NO: 394 | UCACAUGCUUCCGGGAAGG[dT][dT] | 21 nt |
| RAG-698 | SEQ ID NO: 395 | UCCUUCCCGGAAGCAUGUG[dT][dT] | 21 nt |
| | SEQ ID NO: 396 | CACAUGCUUCCGGGAAGGA[dT][dT] | 21 nt |
| RAG-699 | SEQ ID NO: 397 | CUCCUUCCCGGAAGCAUGU[dT][dT] | 21 nt |
| | SEQ ID NO: 398 | ACAUGCUUCCGGGAAGGAG[dT][dT] | 21 nt |
| RAG-700 | SEQ ID NO: 399 | CCUCCUUCCCGGAAGCAUG[dT][dT] | 21 nt |
| | SEQ ID NO: 400 | CAUGCUUCCGGGAAGGAGG[dT][dT] | 21 nt |
| RAG-701 | SEQ ID NO: 401 | CCCUCCUUCCCGGAAGCAU[dT][dT] | 21 nt |
| | SEQ ID NO: 402 | AUGCUUCCGGGAAGGAGGG[dT][dT] | 21 nt |
| RAG-702 | SEQ ID NO: 403 | UCCCUCCUUCCCGGAAGCA[dT][dT] | 21 nt |
| | SEQ ID NO: 404 | UGCUUCCGGGAAGGAGGGA[dT][dT] | 21 nt |
| RAG-704 | SEQ ID NO: 405 | AUUCCCUCCUUCCCGGAAG[dT][dT] | 21 nt |
| | SEQ ID NO: 406 | CUUCCGGGAAGGAGGGAAU[dT][dT] | 21 nt |
| RAG-705 | SEQ ID NO: 407 | AAUUCCCUCCUUCCCGGAA[dT][dT] | 21 nt |
| | SEQ ID NO: 408 | UUCCGGGAAGGAGGGAAUU[dT][dT] | 21 nt |
| RAG-710 | SEQ ID NO: 409 | UCUCCAAUUCCCUCCUUCC[dT][dT] | 21 nt |
| | SEQ ID NO: 410 | GGAAGGAGGGAAUUGGAGA[dT][dT] | 21 nt |
| RAG-711 | SEQ ID NO: 411 | CUCUCCAAUUCCCUCCUUC[dT][dT] | 21 nt |
| | SEQ ID NO: 412 | GAAGGAGGGAAUUGGAGAG[dT][dT] | 21 nt |
| RAG-712 | SEQ ID NO: 413 | UCUCUCCAAUUCCCUCCUU[dT][dT] | 21 nt |
| | SEQ ID NO: 414 | AAGGAGGGAAUUGGAGAGA[dT][dT] | 21 nt |
| RAG-713 | SEQ ID NO: 415 | GUCUCUCCAAUUCCCUCCU[dT][dT] | 21 nt |
| | SEQ ID NO: 416 | AGGAGGGAAUUGGAGAGAC[dT][dT] | 21 nt |
| RAG-714 | SEQ ID NO: 417 | AGUCUCUCCAAUUCCCUCC[dT][dT] | 21 nt |
| | SEQ ID NO: 418 | GGAGGGAAUUGGAGAGACU[dT][dT] | 21 nt |
| RAG-715 | SEQ ID NO: 419 | UAGUCUCUCCAAUUCCCUC[dT][dT] | 21 nt |
| | SEQ ID NO: 420 | GAGGGAAUUGGAGAGACUA[dT][dT] | 21 nt |
| RAG-716 | SEQ ID NO: 421 | GUAGUCUCUCCAAUUCCCU[dT][dT] | 21 nt |
| | SEQ ID NO: 422 | AGGGAAUUGGAGAGACUAC[dT][dT] | 21 nt |
| RAG-717 | SEQ ID NO: 423 | GGUAGUCUCUCCAAUUCCC[dT][dT] | 21 nt |
| | SEQ ID NO: 424 | GGGAAUUGGAGAGACUACC[dT][dT] | 21 nt |
| Hotspot 1 | | | |
| RAG-820 | SEQ ID NO: 425 | UGCAACCACAGGGAUUUCU[dT][dT] | 21 nt |
| | SEQ ID NO: 426 | AGAAAUCCCUGUGGUUGCA[dT][dT] | 21 nt |
| RAG-821 | SEQ ID NO: 427 | CUGCAACCACAGGGAUUUC[dT][dT] | 21 nt |
| | SEQ ID NO: 428 | GAAAUCCCUGUGGUUGCAG[dT][dT] | 21 nt |
| RAG-822 | SEQ ID NO: 429 | GCUGCAACCACAGGGAUUU[dT][dT] | 21 nt |
| | SEQ ID NO: 430 | AAAUCCCUGUGGUUGCAGC[dT][dT] | 21 nt |
| RAG-823 | SEQ ID NO: 431 | UGCUGCAACCACAGGGAUU[dT][dT] | 21 nt |
| | SEQ ID NO: 432 | AAUCCCUGUGGUUGCAGCA[dT][dT] | 21 nt |
| RAG-824 | SEQ ID NO: 433 | CUGCUGCAACCACAGGGAU[dT][dT] | 21 nt |
| | SEQ ID NO: 434 | AUCCCUGUGGUUGCAGCAG[dT][dT] | 21 nt |
| RAG-825 | SEQ ID NO: 435 | GCUGCUGCAACCACAGGGA[dT][dT] | 21 nt |
| | SEQ ID NO: 436 | UCCCUGUGGUUGCAGCAGC[dT][dT] | 21 nt |
| RAG-826 | SEQ ID NO: 437 | AGCUGCUGCAACCACAGGG[dT][dT] | 21 nt |
| | SEQ ID NO: 438 | CCCUGUGGUUGCAGCAGCU[dT][dT] | 21 nt |
| RAG-828 | SEQ ID NO: 439 | AAAGCUGCUGCAACCACAG[dT][dT] | 21 nt |
| | SEQ ID NO: 440 | CUGUGGUUGCAGCAGCUUU[dT][dT] | 21 nt |
| RAG-829 | SEQ ID NO: 441 | CAAAGCUGCUGCAACCACA[dT][dT] | 21 nt |
| | SEQ ID NO: 442 | UGUGGUUGCAGCAGCUUUG[dT][dT] | 21 nt |
| RAG-830 | SEQ ID NO: 443 | ACAAAGCUGCUGCAACCAC[dT][dT] | 21 nt |
| | SEQ ID NO: 444 | GUGGUUGCAGCAGCUUUGU[dT][dT] | 21 nt |
| RAG-831 | SEQ ID NO: 445 | AACAAAGCUGCUGCAACCA[dT][dT] | 21 nt |
| | SEQ ID NO: 446 | UGGUUGCAGCAGCUUUGUU[dT][dT] | 21 nt |
| RAG-832 | SEQ ID NO: 447 | CAACAAAGCUGCUGCAACC[dT][dT] | 21 nt |
| | SEQ ID NO: 448 | GGUUGCAGCAGCUUUGUUG[dT][dT] | 21 nt |
| RAG-833 | SEQ ID NO: 449 | CCAACAAAGCUGCUGCAAC[dT][dT] | 21 nt |
| | SEQ ID NO: 450 | GUUGCAGCAGCUUUGUUGG[dT][dT] | 21 nt |
| RAG-834 | SEQ ID NO: 451 | GCCAACAAAGCUGCUGCAA[dT][dT] | 21 nt |
| | SEQ ID NO: 452 | UUGCAGCAGCUUUGUUGGC[dT][dT] | 21 nt |
| RAG-835 | SEQ ID NO: 453 | GGCCAACAAAGCUGCUGCA[dT][dT] | 21 nt |
| | SEQ ID NO: 454 | UGCAGCAGCUUUGUUGGCC[dT][dT] | 21 nt |
| RAG-836 | SEQ ID NO: 455 | UGGCCAACAAAGCUGCUGC[dT][dT] | 21 nt |
| | SEQ ID NO: 456 | GCAGCAGCUUUGUUGGCCA[dT][dT] | 21 nt |
| RAG-837 | SEQ ID NO: 457 | CUGGCCAACAAAGCUGCUG[dT][dT] | 21 nt |
| | SEQ ID NO: 458 | CAGCAGCUUUGUUGGCCAG[dT][dT] | 21 nt |
| RAG-838 | SEQ ID NO: 459 | CCUGGCCAACAAAGCUGCU[dT][dT] | 21 nt |
| | SEQ ID NO: 460 | AGCAGCUUUGUUGGCCAGG[dT][dT] | 21 nt |
| RAG-840 | SEQ ID NO: 461 | UUCCUGGCCAACAAAGCUG[dT][dT] | 21 nt |
| | SEQ ID NO: 462 | CAGCUUUGUUGGCCAGGAA[dT][dT] | 21 nt |
| RAG-841 | SEQ ID NO: 463 | CUUCCUGGCCAACAAAGCU[dT][dT] | 21 nt |
| | SEQ ID NO: 464 | AGCUUUGUUGGCCAGGAAG[dT][dT] | 21 nt |
| RAG-843 | SEQ ID NO: 465 | CCCUUCCUGGCCAACAAAG[dT][dT] | 21 nt |
| | SEQ ID NO: 466 | CUUUGUUGGCCAGGAAGGG[dT][dT] | 21 nt |
| RAG-844 | SEQ ID NO: 467 | CCCCUUCCUGGCCAACAAA[dT][dT] | 21 nt |
| | SEQ ID NO: 468 | UUUGUUGGCCAGGAAGGGG[dT][dT] | 21 nt |
| RAG-845 | SEQ ID NO: 469 | UCCCCUUCCUGGCCAACAA[dT][dT] | 21 nt |
| | SEQ ID NO: 470 | UUGUUGGCCAGGAAGGGGA[dT][dT] | 21 nt |
| RAG-846 | SEQ ID NO: 471 | CUCCCCUUCCUGGCCAACA[dT][dT] | 21 nt |
| | SEQ ID NO: 472 | UGUUGGCCAGGAAGGGGAG[dT][dT] | 21 nt |
| RAG-848 | SEQ ID NO: 473 | UCCUCCCCUUCCUGGCCAA[dT][dT] | 21 nt |
| | SEQ ID NO: 474 | UUGGCCAGGAAGGGGAGGA[dT][dT] | 21 nt |
| RAG-849 | SEQ ID NO: 475 | AUCCUCCCCUUCCUGGCCA[dT][dT] | 21 nt |
| | SEQ ID NO: 476 | UGGCCAGGAAGGGGAGGAU[dT][dT] | 21 nt |
| RAG-853 | SEQ ID NO: 477 | UCAAAUCCUCCCCUUCCUG[dT][dT] | 21 nt |
| | SEQ ID NO: 478 | CAGGAAGGGGAGGAUUUGA[dT][dT] | 21 nt |
| RAG-854 | SEQ ID NO: 479 | GUCAAAUCCUCCCCUUCCU[dT][dT] | 21 nt |
| | SEQ ID NO: 480 | AGGAAGGGGAGGAUUUGAC[dT][dT] | 21 nt |
| RAG-855 | SEQ ID NO: 481 | CGUCAAAUCCUCCCCUUCC[dT][dT] | 21 nt |
| | SEQ ID NO: 482 | GGAAGGGGAGGAUUUGACG[dT][dT] | 21 nt |
| RAG-856 | SEQ ID NO: 483 | UCGUCAAAUCCUCCCCUUC[dT][dT] | 21 nt |
| | SEQ ID NO: 484 | GAAGGGGAGGAUUUGACGA[dT][dT] | 21 nt |
| RAG-857 | SEQ ID NO: 485 | CUCGUCAAAUCCUCCCCUU[dT][dT] | 21 nt |
| | SEQ ID NO: 486 | AAGGGGAGGAUUUGACGAG[dT][dT] | 21 nt |
| RAG-858 | SEQ ID NO: 487 | ACUCGUCAAAUCCUCCCCU[dT][dT] | 21 nt |
| | SEQ ID NO: 488 | AGGGGAGGAUUUGACGAGU[dT][dT] | 21 nt |
| RAG-860 | SEQ ID NO: 489 | UCACUCGUCAAAUCCUCCC[dT][dT] | 21 nt |
| | SEQ ID NO: 490 | GGGAGGAUUUGACGAGUGA[dT][dT] | 21 nt |
| RAG-861 | SEQ ID NO: 491 | CUCACUCGUCAAAUCCUCC[dT][dT] | 21 nt |
| | SEQ ID NO: 492 | GGAGGAUUUGACGAGUGAG[dT][dT] | 21 nt |
| RAG-862 | SEQ ID NO: 493 | ACUCACUCGUCAAAUCCUC[dT][dT] | 21 nt |
| | SEQ ID NO: 494 | GAGGAUUUGACGAGUGAGU[dT][dT] | 21 nt |
| RAG-864 | SEQ ID NO: 495 | CAACUCACUCGUCAAAUCC[dT][dT] | 21 nt |
| | SEQ ID NO: 496 | GGAUUUGACGAGUGAGUUG[dT][dT] | 21 nt |
| RAG-865 | SEQ ID NO: 497 | ACAACUCACUCGUCAAAUC[dT][dT] | 21 nt |
| | SEQ ID NO: 498 | GAUUUGACGAGUGAGUUGU[dT][dT] | 21 nt |
| RAG-866 | SEQ ID NO: 499 | GACAACUCACUCGUCAAAU[dT][dT] | 21 nt |
| | SEQ ID NO: 500 | AUUUGACGAGUGAGUUGUC[dT][dT] | 21 nt |
| RAG-867 | SEQ ID NO: 501 | AGACAACUCACUCGUCAAA[dT][dT] | 21 nt |
| | SEQ ID NO: 502 | UUUGACGAGUGAGUUGUCU[dT][dT] | 21 nt |
| RAG-868 | SEQ ID NO: 503 | CAGACAACUCACUCGUCAA[dT][dT] | 21 nt |
| | SEQ ID NO: 504 | UUGACGAGUGAGUUGUCUG[dT][dT] | 21 nt |
| RAG-869 | SEQ ID NO: 505 | ACAGACAACUCACUCGUCA[dT][dT] | 21 nt |
| | SEQ ID NO: 506 | UGACGAGUGAGUUGUCUGU[dT][dT] | 21 nt |
| RAG-870 | SEQ ID NO: 507 | GACAGACAACUCACUCGUC[dT][dT] | 21 nt |
| | SEQ ID NO: 508 | GACGAGUGAGUUGUCUGUC[dT][dT] | 21 nt |
| RAG-871 | SEQ ID NO: 509 | AGACAGACAACUCACUCGU[dT][dT] | 21 nt |
| | SEQ ID NO: 510 | ACGAGUGAGUUGUCUGUCU[dT][dT] | 21 nt |
| RAG-872 | SEQ ID NO: 511 | GAGACAGACAACUCACUCG[dT][dT] | 21 nt |
| | SEQ ID NO: 512 | CGAGUGAGUUGUCUGUCUC[dT][dT] | 21 nt |
| RAG-873 | SEQ ID NO: 513 | GGAGACAGACAACUCACUC[dT][dT] | 21 nt |
| | SEQ ID NO: 514 | GAGUGAGUUGUCUGUCUCC[dT][dT] | 21 nt |
| RAG-874 | SEQ ID NO: 515 | AGGAGACAGACAACUCACU[dT][dT] | 21 nt |
| | SEQ ID NO: 516 | AGUGAGUUGUCUGUCUCCU[dT][dT] | 21 nt |
| RAG-875 | SEQ ID NO: 517 | CAGGAGACAGACAACUCAC[dT][dT] | 21 nt |
| | SEQ ID NO: 518 | GUGAGUUGUCUGUCUCCUG[dT][dT] | 21 nt |
| RAG-876 | SEQ ID NO: 519 | UCAGGAGACAGACAACUCA[dT][dT] | 21 nt |
| | SEQ ID NO: 520 | UGAGUUGUCUGUCUCCUGA[dT][dT] | 21 nt |
| RAG-877 | SEQ ID NO: 521 | UUCAGGAGACAGACAACUC[dT][dT] | 21 nt |
| | SEQ ID NO: 522 | GAGUUGUCUGUCUCCUGAA[dT][dT] | 21 nt |
| RAG-878 | SEQ ID NO: 523 | AUUCAGGAGACAGACAACU[dT][dT] | 21 nt |
| | SEQ ID NO: 524 | AGUUGUCUGUCUCCUGAAU[dT][dT] | 21 nt |
| RAG-879 | SEQ ID NO: 525 | UAUUCAGGAGACAGACAAC[dT][dT] | 21 nt |
| | SEQ ID NO: 526 | GUUGUCUGUCUCCUGAAUA[dT][dT] | 21 nt |
| RAG-880 | SEQ ID NO: 527 | GUAUUCAGGAGACAGACAA[dT][dT] | 21 nt |
| | SEQ ID NO: 528 | UUGUCUGUCUCCUGAAUAC[dT][dT] | 21 nt |
| RAG-881 | SEQ ID NO: 529 | AGUAUUCAGGAGACAGACA[dT][dT] | 21 nt |
| | SEQ ID NO: 530 | UGUCUGUCUCCUGAAUACU[dT][dT] | 21 nt |
| RAG-882 | SEQ ID NO: 531 | GAGUAUUCAGGAGACAGAC[dT][dT] | 21 nt |
| | SEQ ID NO: 532 | GUCUGUCUCCUGAAUACUC[dT][dT] | 21 nt |
| RAG-883 | SEQ ID NO: 533 | GGAGUAUUCAGGAGACAGA[dT][dT] | 21 nt |
| | SEQ ID NO: 534 | UCUGUCUCCUGAAUACUCC[dT][dT] | 21 nt |
| RAG-884 | SEQ ID NO: 535 | GGGAGUAUUCAGGAGACAG[dT][dT] | 21 nt |
| | SEQ ID NO: 536 | CUGUCUCCUGAAUACUCCC[dT][dT] | 21 nt |
| RAG-885 | SEQ ID NO: 537 | GGGGAGUAUUCAGGAGACA[dT][dT] | 21 nt |
| | SEQ ID NO: 538 | UGUCUCCUGAAUACUCCCC[dT][dT] | 21 nt |
| RAG-886 | SEQ ID NO: 539 | UGGGGAGUAUUCAGGAGAC[dT][dT] | 21 nt |
| | SEQ ID NO: 540 | GUCUCCUGAAUACUCCCCA[dT][dT] | 21 nt |
| RAG-887 | SEQ ID NO: 541 | GUGGGGAGUAUUCAGGAGA[dT][dT] | 21 nt |
| | SEQ ID NO: 542 | UCUCCUGAAUACUCCCCAC[dT][dT] | 21 nt |
| RAG-888 | SEQ ID NO: 543 | UGUGGGGAGUAUUCAGGAG[dT][dT] | 21 nt |
| | SEQ ID NO: 544 | CUCCUGAAUACUCCCCACA[dT][dT] | 21 nt |
| RAG-889 | SEQ ID NO: 545 | AUGUGGGGAGUAUUCAGGA[dT][dT] | 21 nt |
| | SEQ ID NO: 546 | UCCUGAAUACUCCCCACAU[dT][dT] | 21 nt |
| RAG-890 | SEQ ID NO: 547 | UAUGUGGGGAGUAUUCAGG[dT][dT] | 21 nt |
| | SEQ ID NO: 548 | CCUGAAUACUCCCCACAUA[dT][dT] | 21 nt |
| RAG-891 | SEQ ID NO: 549 | CUAUGUGGGGAGUAUUCAG[dT][dT] | 21 nt |
| | SEQ ID NO: 550 | CUGAAUACUCCCCACAUAG[dT][dT] | 21 nt |
| RAG-892 | SEQ ID NO: 551 | GCUAUGUGGGGAGUAUUCA[dT][dT] | 21 nt |
| | SEQ ID NO: 552 | UGAAUACUCCCCACAUAGC[dT][dT] | 21 nt |
| RAG-893 | SEQ ID NO: 553 | GGCUAUGUGGGGAGUAUUC[dT][dT] | 21 nt |
| | SEQ ID NO: 554 | GAAUACUCCCCACAUAGCC[dT][dT] | 21 nt |
| RAG-894 | SEQ ID NO: 555 | GGGCUAUGUGGGGAGUAUU[dT][dT] | 21 nt |
| | SEQ ID NO: 556 | AAUACUCCCCACAUAGCCC[dT][dT] | 21 nt |

These hotspots include: hotspot 1 having a corresponding target sequence from −893 bp to −801 bp in the p21 promoter sequence, shown as SEQ ID NO: 93, wherein 44 functional saRNAs (Table 4, FIG. 3A) were discovered in this region, comprising RAG-834, RAG-845, RAG-892, RAG-846, RAG-821, RAG-884, RAG-864, RAG-843, RAG-854, RAG-844, RAG-887, RAG-838, RAG-858, RAG-835, RAG-876, RAG-870, RAG-853, RAG-881, RAG-828, RAG-872, RAG-841, RAG-831, RAG-829, RAG-820, RAG-822, RAG-868, RAG-849, RAG-862, RAG-865, RAG-893, RAG-848, RAG-824, RAG-866, RAG-840, RAG-875, RAG-880, RAG-871, RAG-888, RAG-885, RAG-894, RAG-833, RAG-825, RAG-889, and RAG-823;

hotspot 2 (Table 4, FIG. 3B) having a corresponding target sequence from −717 bp to −632 bp in the p21 promoter sequence, shown as SEQ ID NO: 94, wherein 31 functional saRNAs were discovered in this region, comprising RAG-693, RAG-692, RAG-688, RAG-696, RAG-694, RAG-687, RAG-691, RAG-690, RAG-689, RAG-682, RAG-686, RAG-662, RAG-695, RAG-654, RAG-658, RAG-685, RAG-704, RAG-714, RAG-705, RAG-661, RAG-656, RAG-698, RAG-697, RAG-657, RAG-715, RAG-652, RAG-651, RAG-650, RAG-716, RAG-717, and RAG-711;
hotspot 3 (table 4, FIG. 3C) having a corresponding target sequence from −585 bp to −551 bp in the p21 promoter sequence, shown as SEQ ID NO: 95, wherein 9 functional saRNAs were discovered in this region, comprising RAG-580, RAG-577, RAG-569, RAG-576, RAG-570, RAG-574, RAG-585, RAG-579, and RAG-584;
hotspot 4 (Table 4, FIG. 3D) having a corresponding target sequence from −554 bp to −505 bp in the p21 promoter sequence, shown as SEQ ID NO: 96, wherein 17 functional saRNAs were discovered in this region, comprising RAG-524, RAG-553, RAG-537, RAG-526, RAG-554, RAG-523, RAG-534, RAG-543, RAG-525, RAG-535, RAG-546, RAG-545, RAG-542, RAG-531, RAG-522, RAG-529, and RAG-552;
hotspot 5 (Table 4, FIG. 3E) having a corresponding target sequence from −514 bp to −485 bp in the p21 promoter sequence, shown as SEQ ID NO: 97, wherein 9 functional saRNAs were discovered in this region, comprising RAG-503, RAG-504, RAG-505, RAG-506, RAG-507, RAG-508, RAG-509, RAG-510, RAG-511, RAG-512, RAG-513, and RAG-514;
hotspot 6 (table 4, FIG. 3F) having a corresponding target sequence from −442 bp to −405 bp in the p21 promoter sequence, shown as SEQ ID NO: 98, wherein 12 functional saRNAs were discovered in this region, comprising RAG-427, RAG-430, RAG-431, RAG-423, RAG-425, RAG-433, RAG-435, RAG-434, RAG-439, RAG-426, RAG-428, and RAG-442;
hotspot 7 (Table 4, FIG. 3G) having a corresponding target sequence from −352 bp to −313 bp in the p21 promoter sequence, shown as SEQ ID NO: 99, wherein 13 functional saRNAs were discovered in this region, comprising RAG-335, RAG-351, RAG-352, RAG-331, RAG-344, RAG-342, RAG-341, RAG-333, RAG-345, RAG-346, RAG-336, RAG-332, and RAG-343;
and hotspot 8 (Table 4, FIG. 3H) having a corresponding target sequence from −325 bp to −260 bp in the p21 promoter sequence, shown as SEQ ID NO: 100, wherein 18 functional saRNAs were discovered in this region, comprising RAG-294, RAG-285, RAG-286, RAG-292, RAG-291, RAG-284, RAG-279, RAG-280, RAG-325, RAG-293, RAG-322, RAG-321, RAG-281, RAG-289, RAG-278, RAG-283, RAG-282, and RAG-295.

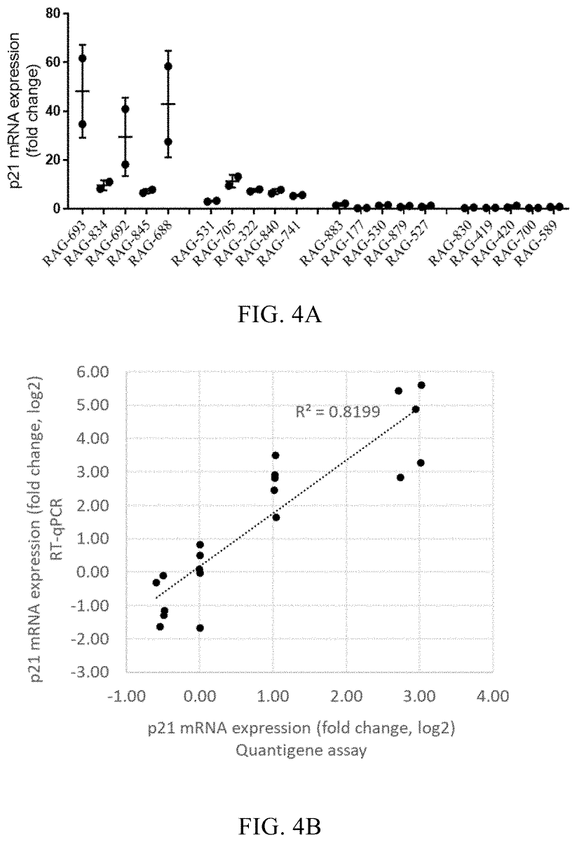

In order to verify the QuantiGene 2.0 assay results, the 439 saRNAs were divided into four bins according to their activity in inducing p21 mRNA expression, and 5 saRNAs were randomly selected from each bin and transfected into Ku-7-luc2-GFP cells at a concentration of 10 nM. 72 hours after transfection, total cellular RNA was extracted from the transfected cells and reverse transcribed into cDNA which was amplified by RT-qPCR to determine p21 mRNA level. p21 mRNA expression levels for cells transfected with each of the saRNA determined by the two methods showed a significant correlation (R²=0.82) (FIG. 4). All selected functional saRNAs obtained through the QuantiGene 2.0 method were verified as real functional saRNAs by the RT-qPCR method, and some of them exhibited even a stronger p21 mRNA induction ability by RT-qPCR (Table 5).

TABLE 5
Verification for QuantiGene 2.0 method

Relative p21 mRNA level

| saRNA group | saRNA | Quantigene 2.0 | RT-qPCR |
|---|---|---|---|
| Bin 1 | RAG-693 | 8.12 | 48.20 |
| | RAG-834 | 8.07 | 9.64 |
| | RAG-692 | 7.69 | 29.53 |
| | RAG-845 | 6.67 | 7.15 |
| | RAG-688 | 6.55 | 42.91 |
| Bin 2 | RAG-531 | 2.06 | 3.11 |
| | RAG-705 | 2.05 | 11.33 |
| | RAG-322 | 2.04 | 7.53 |
| | RAG-840 | 2.03 | 7.01 |
| | RAG-741 | 2.02 | 5.45 |
| Bin 3 | RAG-883 | 1.00 | 1.76 |
| | RAG-177 | 1.00 | 0.31 |
| | RAG-530 | 1.00 | 1.41 |
| | RAG-879 | 1.00 | 0.98 |
| | RAG-527 | 0.99 | 1.06 |
| Bin 4 | RAG-830 | 0.72 | 0.45 |
| | RAG-419 | 0.71 | 0.41 |
| | RAG-420 | 0.71 | 0.93 |
| | RAG-700 | 0.69 | 0.32 |
| | RAG-589 | 0.66 | 0.80 |

Taken together, the above data indicates that saRNAs can be designed to target selected regions in the p21 promoter to induce p21 expression with some regions being more sensitive and containing higher percentages of functional saRNA targets.

### Example 2: saRNAs Induce p21 mRNA Expression and Inhibit Cancer Cell Proliferation

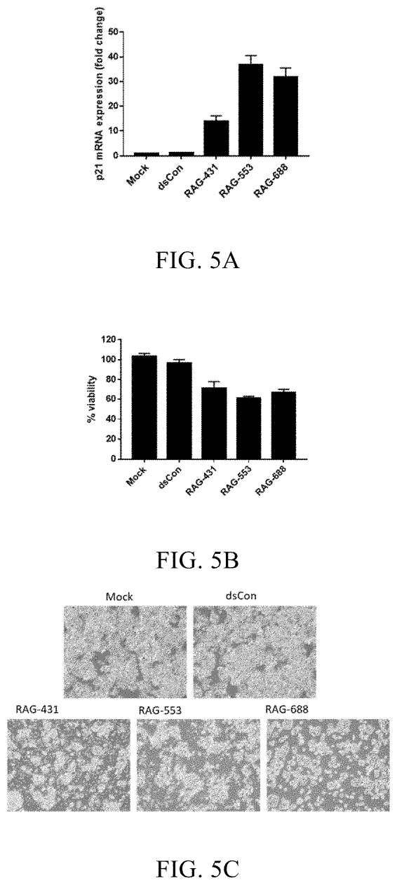

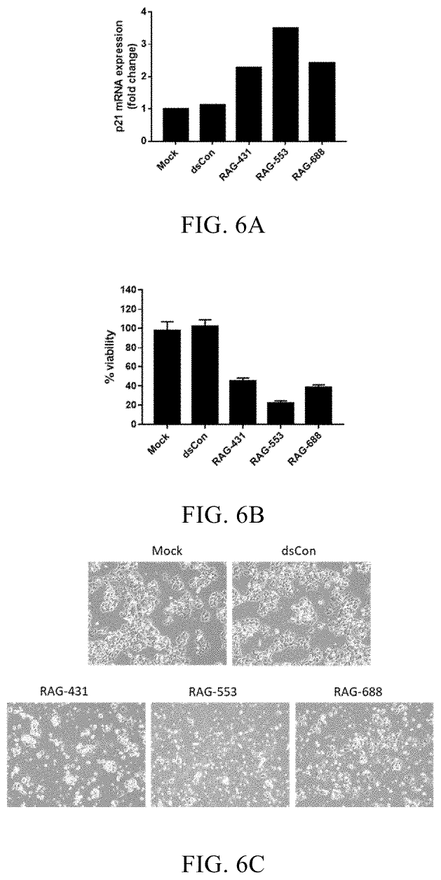

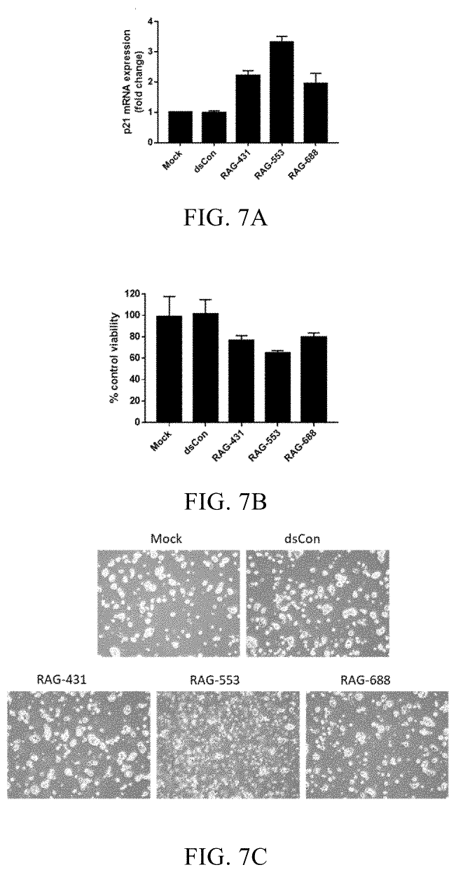

In order to further evaluate the effect of p21 saRNAs in inducing p21 mRNA expression and suppressing cancer cell proliferation, the saRNAs (RAG1-431, RAG1-553 and RAG1-688) screened by QuantiGene 2.0 were transfected into cancer cell lines including Ku-7-luc2-GFP (bladder cancer), HCT116 (colon cancer) and HepG2 (hepatocellular carcinoma). The result showed that in the aforementioned cell lines, all saRNAs could induce at least a two-fold change in the p21 mRNA expression levels and suppress cell proliferation, indicating functional activation of p21 protein. Specifically, RAG-431, RAG-553, and RAG-688 were individually transfected into Ku-7-luc2-GFP cells, caused a 14.0, a 36.9, and a 31.9-fold change in the mRNA expression of p21, and exhibited a 71.7%, 60.7% and 67.4% cell survival rate respectively relative to blank control (Mock) (FIG. 5). RAG-431, RAG-553 and RAG-688 were transfected into the HCT116 cells, resulted in a 2.3, a 3.5, and a 2.4-fold change in the mRNA expression of p21, and exhibited a survival rate of 45.3%, 22.5% and 38.5% respectively relative to blank control (Mock) (FIG. 6). RAG-431, RAG-553, and RAG-688 were transfected into the HepG2 cells, resulted in a 2.2, 3.3, and a 2.0-fold change in the mRNA expression of p21, and exhibited a survival rate of 76.7%, 64.9%, and 79.9% respectively relative to blank control (Mock) (FIG. 7).

### Example 3: Further Screening and Optimizing saRNAs in a Hotspot Region

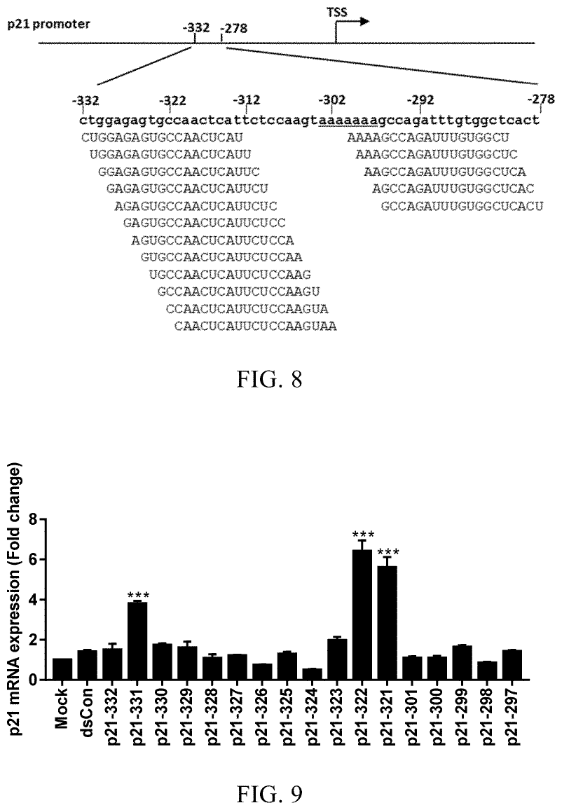

In order to screen and validate p21 activating saRNAs, a more focused screen was performed against hotspot 7 which is a 40 bp region from −332 to −292 relative to the TSS of p21. This region contains 17 overlapping 19-bp saRNA target sites after a short polyadenosine repetitive sequence was excluded (FIG. 8). Seventeen saRNAs with standard structure (19 nt symmetric duplex region with 3′ dTdT overhangs) corresponding to each of the 17 targets were synthesized and named, according to their target positions relative to the TSS, as P21-297, P21-298, P21-299, P21-325, and so on. All the duplex sequences are listed in Table 1. Each of the duplexes was transfected into Ku-7-luc2-GFP (bladder urothelial carcinoma) cells individually, and 72 hours after transfection, p21 mRNA expression levels were detected. As shown in FIG. 9, multiple duplexes (i.e. P21-321, P21-322, and P21-331) are strong activators for p21, resulting in about 4- to 6-fold changes in p21 expression level, while the other duplexes did not significantly upregulate p21 expression. These data showed that the gene activation effect of a saRNA highly depends on the target location and/or their sequence thereof and a small shift of the target position (for example, one bp shift) can make a functional saRNA unfunctional.

### Example 4: Duplex saRNAs with 3′ Natural Overhang or 2-Nucleotide Uracil Overhang

In addition, the potency of a specific saRNA can be further improved by optimizing the design of the saRNA structure. Supporting data are as follows:

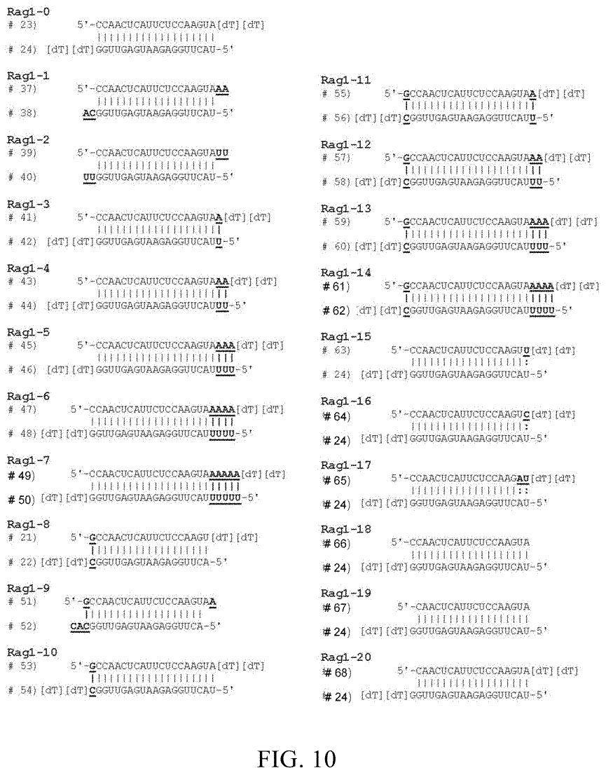

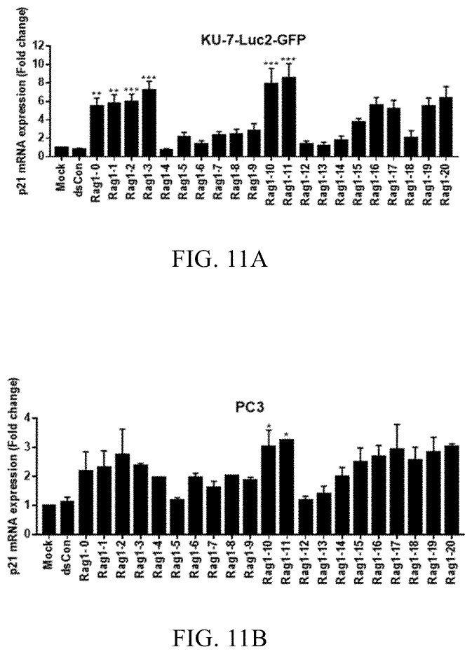

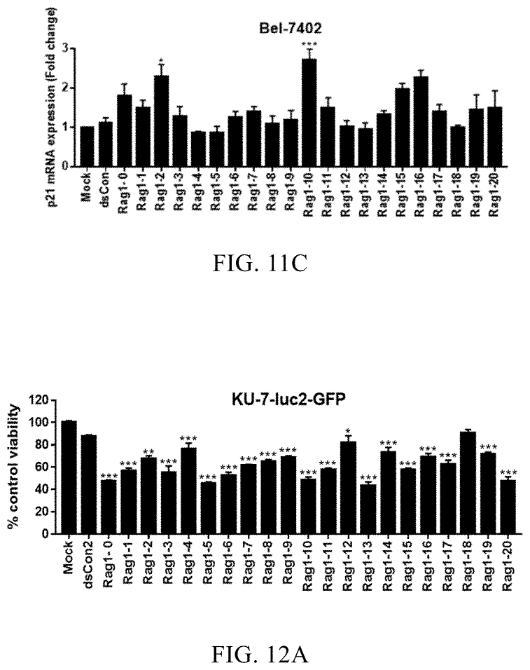

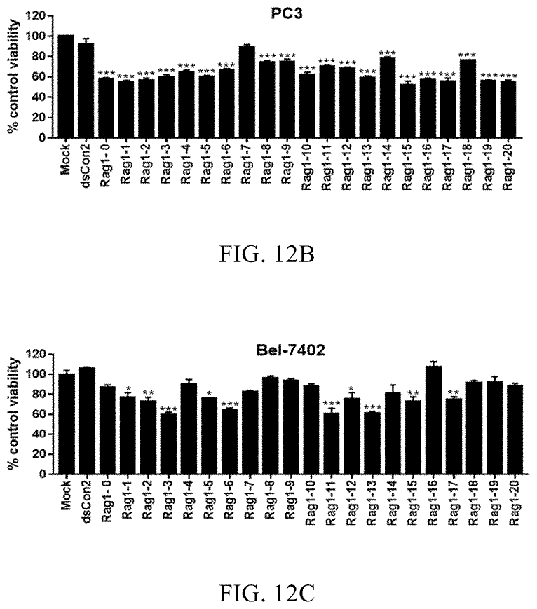

P21-322 (referred to as Rag1-0 hereinafter) was selected for further development with novel sequences and structures to improve its drug-like properties. First, duplex saRNA with different overhangs were tested including replacing dTdT overhang on each strand by a natural nucleotide overhang (i.e. homologous to the corresponding nucleotides on the target) or a UU overhang, resulting in two new saRNA variants (named as Rag1-1 and Rag1-2 respectively) (FIG. 10). Each of the two duplex saRNAs was transfected into three different types of cell lines, including Ku-7-luc2-GFP, PC3 (prostate cancer), and Bel-7402 (hepatocellular carcinoma) cells. 72 hours after transfection, p21 mRNA expression levels were evaluated. As shown in FIGS. 11A-11C, in Ku-7-luc2-GFP cells, the p21 induction effect of Rag1-1 and Rag1-2 was slightly higher than that of Rag1-0; and in PC3 and Bel-7402 cells, the p21 induction effect of Rag1-2 is stronger than that of Rag1-0. The cell viability data as measured by the CCK-8 assay showed that the suppressing effects of both Rag1-1 and Rag1-2 against Bel7402 cell proliferation were enhanced compared with that of Rag1-0 (FIG. 12). These data indicated that saRNA duplexes with different overhang structures, i.e. natural nucleotide overhangs and UU overhangs, were not functionally inferior to Rag1-0, and had a better effect in inducing p21 expression and suppressing cell proliferation than Rag1-0 for certain cell types. This also suggested that in certain cancer cell types, the natural nucleotide overhang and the UU overhang structure improved the activity of a saRNA in gene induction and cell growth suppression. Duplex saRNAs with a natural overhang are 100% complementary with the target sequence, and its entire sequence is derived from its target site, which differs from dTdT and UU overhangs. This design provided a better target specificity and complementarity compared with saRNAs containing unnatural or mispaired overhangs.

### Example 5: Effects of Duplex saRNA Sizes on saRNA's Activity in Inducing p21

In order to determine whether duplex saRNA length can influence saRNA activity in inducing p21 expression, 5 novel duplexes were designed by extending the Rag1-0 target, one nucleotide at a time from 19 nt to 24 nt toward the direction of the p21 TSS and named Rag1-3, Rag1-4, Rag1-5, Rag1-6, and Rag1-7, which are of 22, 23, 24, 25 and 26 nucleotides in size after adding dTdT overhangs to the 3′ end of each strand in the duplexes, (FIG. 10). As shown in FIG. 11A-11C, Rag1-3, which was 22 nucleotides in length, exhibited stronger activity in inducing p21 gene expression and inhibiting cell proliferation in Ku-7-luc2-GFP cells compared with Rag1-0 (FIG. 12). However, duplexes longer than 22 nucleotides lost their activity, suggesting that the duplex length of 21 to 22 nucleotides was preferable for gene induction.

Further, on the basis of P21-323 (also named as Rag1-8), a variant (Rag1-9) containing natural nucleotide overhangs and variants of 22, 23, 24, 25, and 26 nucleotides in length with 3′ dTdT overhangs (named as Rag1-10, Rag1-11, Rag1-12, Rag1-13, and Rag1-14, respectively) were designed and synthesized (FIG. 10). As shown in FIG. 11A-11C, Rag1-9, which is 21 nucleotides in length, and bears natural overhangs did not show improved activity, while Rag1-10, which is 22 nucleotides in length, exhibited higher activity compared to all other duplexes in the cell lines tested, and, therefore, was a strong p21 activator. Meanwhile, Rag1-11, which is 23 nucleotides in length, exhibited an activity in Ku-7-luc2-GFP and PC3 cells comparable to Rag1-10, but not in Bel-7402 cells (FIG. 11A-11C). Therefore, extending the length of a saRNA could transform an unfunctional one into a potent gene activator.

### Example 6: Duplex saRNAs with Mispaired Bases

The applicants designed and synthesized variants of Rag1-0, wherein the adenine nucleotide (A) of the sense strand corresponding to the first nucleotide counted from the 5′ terminus of the antisense strand was mutated into a uracil and cytosine, but the antisense strand was kept unchanged, to obtain two new saRNA duplexes Rag1-15 and Rag1-16, respectively, which contained a single mispaired base pair at the last basepair of the duplex. In addition, the nucleotides of the sense strand corresponding to the first and second nucleotide counted from the 5′ terminus of the antisense strand were mutated into an adenosine nucleotide and a uracil nucleotide respectively to obtain a saRNA variant, Rag1-17, which contains two mispaired bases at the 3′ terminus of the duplex (FIG. 10). These saRNAs were transfected into Ku-7-luc2-GFP, PC3, or Bel-7402 cells and the result showed that all three saRNA variants had better or at least equivalent activity in inducing p21 expression compared with Rag1-0. However, saRNA variant Rag1-16 that contained a mispaired cytosine was the strongest activator in all three cell lines (FIG. 11A-11C).

### Example 7: Duplex saRNAs with Asymmetrical Structures

Asymmetrically structured duplexes were also synthesized, including Rag1-18 (having a 3′ blunt-end instead of an overhang at the 3′ terminus of the sense strand), Rag1-20 (having a 3-nucleotide overhang at the 3′ terminus of the antisense strand due to the removal of the first nucleotide counted from the 5′ terminus of the sense strand), and Rag1-19 (containing a combination of the modifications in Rag1-18 and Rag1-20, i.e., an asymmetric duplex structure characteristic of a 5′ end 3-nucleotide overhang and a 3′ blunt end which was formed by removing the first nucleotide at the 5′ terminus and the dTdT overhang at the 3′ terminus of the sense strand) (FIG. 10). As shown in FIG. 11A-11C, Rag1-18 failed to activate p21 in all three cell lines, whereas Rag1-20 was capable of inducing p21 expression in all three cell lines to a similar extent as Rag1-0. Compared to Rag1-18, the combination of the two modifications in Rag1-19 led to the recovery of p21 inducing activity lost in Rag1-18. This data supports that these two structural optimizations does not impair gene induction activity while potentially offering an advantage of reduced off-target effects from the sense strand. An extra advantage is the reduced cost for oligonucleotide synthesis due to shortened nucleotides.

### Example 8: saRNA Optimization by Different Combinations of Structural Features

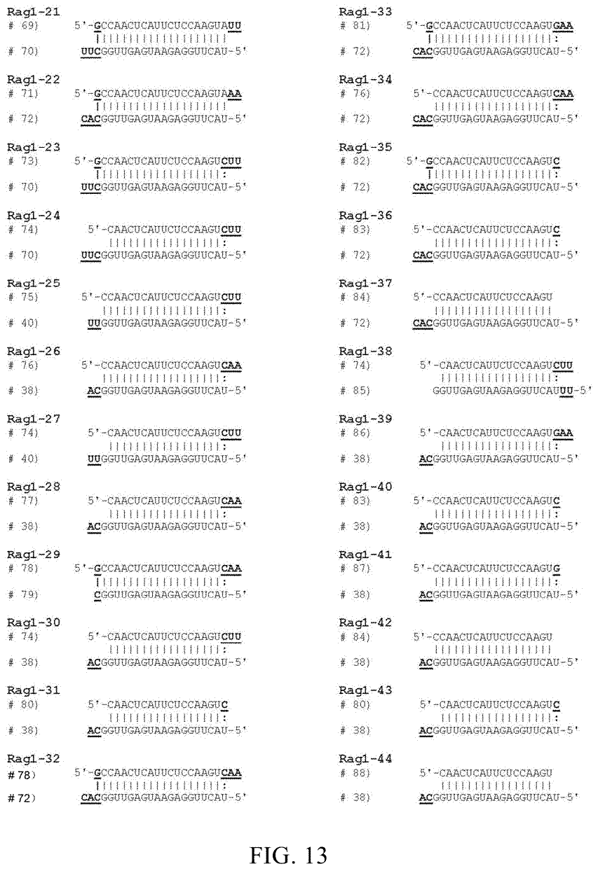

1. As shown in FIG. 13, Rag1-21, Rag1-22, Rag1-23, and Rag1-24 were derivatives of Rag1-10.
(1) Rag1-21 contains UU overhangs and has the following structural formula: sense strand (S₂₀+3U₂) and antisense strand (AS₂₀+3U₂).
(2) Rag1-22 contains 2-nt natural nucleotide overhangs, and its structural combination formula is: sense strand (S_{zo}+3NO₂) and antisense strand (AS₂₀+3NO₂).
(3) Rag1-23 is a variant of Rag1-21 and contains a mismatch at the first base counted from the 3′ terminus of the duplex which was created by mutating the adenine of the sense strand at that position into a cytosine. The structural combination formula is: sense strand (S₂₀+3U₂), antisense strand (AS₂₀+3U₂), and duplex (MM_{(3′1S:A, C)}).
(4) Rag1-24 is a variant of Rag1-23, wherein the first nucleotide at the 5′ terminus of the sense strand was deleted, and the length of the overhang of the antisense strand was thus increased. The structural combination formula is: sense strand (S₁₉+3U₂), antisense strand (AS₂₀+3U₂NO₂), and duplex (MM_{(3′1S:A, C)}).

2. Rag1-25 and Rag1-26 were variants of Rag1-16, wherein overhangs of the sense strand and the antisense strand were replaced by a UU overhang and a natural overhang respectively, wherein the first nucleotide at the 3′ terminus of the sense strand was mutated to a cytosine to create a mismatch.
(1) The structural combination formula of Rag1-25 is: sense strand (S₁₉+3U₂), antisense strand (AS₁₉+3U₂), and duplex (MM_{(3′1S:A, C)}).
(2) The structural combination formula of Rag1-26 is: sense strand (S₁₉+³NO₂), antisense strand (AS₁₉+3NO₂), and duplex (MM_{(3′1S:A, C)})).

3. Rag1-27 and Rag1-28 were variants of Rag1-20, and in both variants, the first nucleotide at the 3′ terminus of the sense strand was mutated to a cytosine to create a mismatch and the first nucleotide at the 5′ terminus was deleted, resulting in a 3-nt overhang in the antisense strand. The sense and antisense strands are 20 and 21 nucleotides in length respectively.
(1) Rag1-27 contains UU overhangs and its structural combination formula is: sense strand (S₁₈+3U₂), antisense strand (AS₁₉+3U₂), and duplex (MM_{(3′1S:A, C)})).
(2) Rag1-28 contains 2-nt natural nucleotide overhangs and its structural combination formula is: sense strand (S₁₈+3NO₂), antisense strand (AS₁₉+3NO₂), and duplex (MM_{(3′1S:A, C)})).

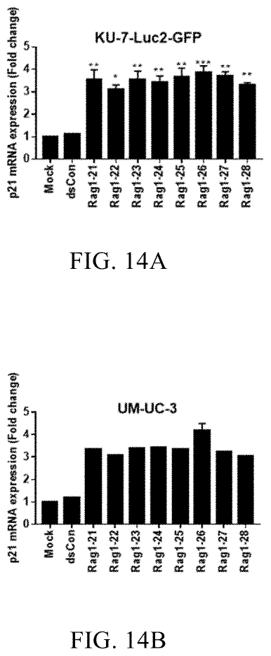

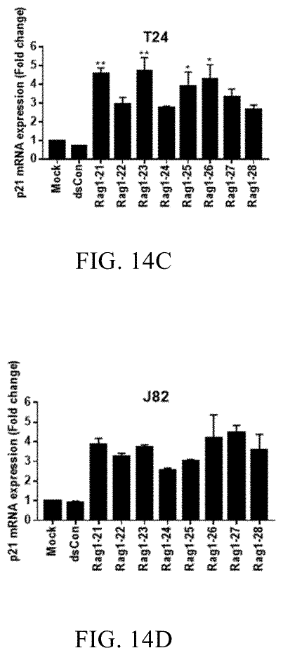

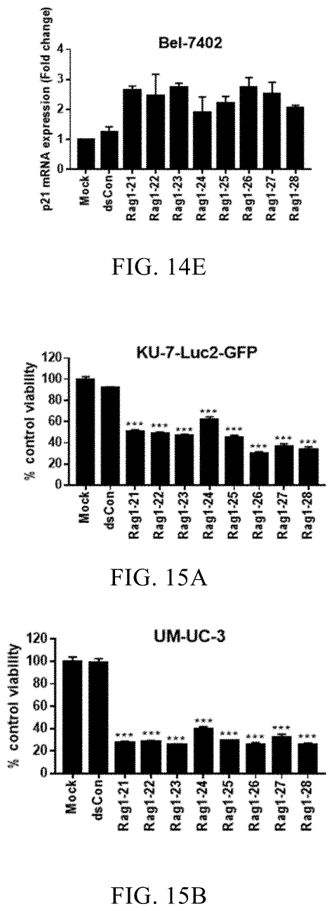

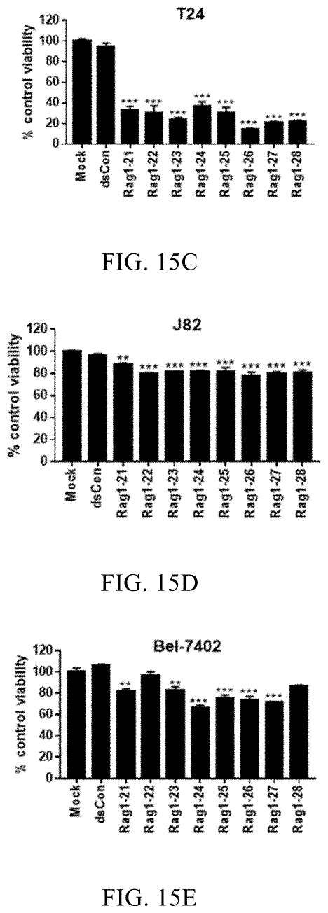

These saRNA variants (Rag1-21 to Rag1-28) were transfected into Ku-7-luc2-GFP, UM-UC-3 (bladder transitional cell carcinoma), T24 (bladder cancer), J82 (bladder transitional cell carcinoma), or Bel-7402 cell lines, and 72 hours after transfection, p21 gene expression was evaluated. As shown in FIG. 14A-14E, all duplexes induced p21 expression to different extents, of which, Rag1-21, Rag1-23, and Rag1-26 performed better than other variants. Meanwhile, the proliferative capabilities of the transfected cells were analyzed by CCK-8 assay, and the results showed that these saRNA variants exhibited varying degrees of growth inhibition in all cells with Rag1-26 performing the best in Ku-7-luc2-GFP, UM-UC-3, and T24 cells (FIG. 15A-15E). These data were indicative of the benefits provided by the cytosine mismatch at the 3′ end of the duplexes and the natural overhang structure.

3. On the basis of Rag1-26, novel variants Rag1-29 to Rag1-44 were designed (Table 1, FIG. 13), and these variants contained the different combinations of the following sequence and structural tweaks:

strand length variations:
i. a sense strand of 17 to 22 nucleotides in length;
ii. an antisense strand of 20 to 22 nucleotides in length;

duplex structure variations:
iii. 5′ blunt end;
iv. 3′ blunt end;
v. mispairing;

overhang feature variations:
vi. a 1- to 3-nucleotide overhang at the 3′ terminus of the antisense strand;
vii. a 1- to 2-nucleotide overhang at the 5′ terminus of the antisense strand;
viii. a 2-nucleotide (natural nucleotide or UU) overhang at the 3′ terminus of the sense strand.

(1) Rag1-29 had an asymmetric structure with a 5′ blunt end which was achieved by removing the overhang of the antisense strand, the purpose of which was to determine whether the overhang of the antisense strand was necessary for p21 induction, and the structural combination formula is: sense strand (S₂₀+3NO₂), antisense strand (AS₂₀), and duplex (B₅+MM_{(3′1S:A, C)}).

(2) Rag1-30 had a sense strand with its first 5′ terminus nucleotide truncated and a UU overhang at the 3′ terminus, and an antisense strand with a 3-nt natural nucleotides overhang at the 3′ terminus. The structural combination formula is: sense strand (S₁₈+3U₂), antisense strand (AS₁₈+3NO₃), and duplex (MM_{(3′1S:A, C)})).

(3) Rag1-31 was similar to Rag1-30 but had an asymmetric structure with a blunt end formed by removing the overhang of the sense strand. The structural combination formula is: sense strand (S₁₈), antisense strand (AS₁₈ 3 NO₃), and duplex (MM_{(3′1S:A, C)}).

(4) Rag1-32 was similar to Rag1-29 but had a UU overhang at the 3′ terminus of the antisense strand, instead of a natural overhang. The structural combination formula is: sense strand (S₂₀+3 NO₂), antisense strand (AS₂₀+3NO₂), and duplex (MM_{(3′1S:A, C)}).

(5) Rag1-33 was similar to Rag1-32, except that the mispaired cytosine nucleotide in the sense strand was replaced by a guanine nucleotide to test the effect of G:U “wobble” base pairing. The structural combination formula is: sense strand (S₂₀+3NO₂), antisense strand (AS₂₀+3NO₂), and duplex (MM_{(3′1S:A, G)}).

(6) Rag1-34 was similar to Rag1-32 but had a nucleotide deletion at the 5′ terminus of the sense strand. The structural combination formula is: sense strand (S₁₉+3NO₂), antisense strand (AS₁₉ 3 NO₃), and duplex (MM_{(3′1S:A, C)}).

(7) Rag1-35 was a blunt-end derivative of Rag1-32, wherein the overhang of the sense strand was removed. The structural combination formula is: sense strand (S₂₀), antisense strand (AS₂₀+3NO₂) and duplex (B3+MM_{(3′1S:A, C)}).

(8) Rag1-36 was similar to Rag1-35, and had an asymmetric structure with a blunt end, formed by deleting a nucleotide at the 5′ terminus of the sense strand. The structural combination formula is: sense strand (S₁₉), antisense strand (AS₁₉ 3 NO₃), and duplex (B3+MM_{(3′1S:A, C)}).

(9) Rag1-37 was similar to Rag1-36, except that the mispaired cytosine nucleotide was deleted from the sense strand, resulting in a single-nucleotide overhang at the 5′ terminus of the antisense strand. The structural combination formula is: sense strand (S₁₈), antisense strand (5NO₁+AS₁₈+3NO₃), and duplex (OH_{As5′}).

(10) Rag1-38 was an unconventional derivative of Rag 1-27, wherein the antisense strand possessed a UU overhang at its 5′ terminus to match the 3′ overhang of the sense strand and a single-nucleotide overhang at the 3′ terminus. The structural combination formula is: sense strand (S₁₈+3U₂), antisense strand (5U₂+AS₁₈+3NO₁), and duplex (OH_{AS5′}+MM_{(3′1S:A, C)}).

(11) Rag1-39 was a derivative of Rag1-33, wherein the mispaired cytosine nucleotide of the sense strand was replaced by a guanine nucleotide, and the duplex length was reduced by one base pair. The structural combination formula is: sense strand (S₁₈+3NO₂), antisense strand (AS₁₈ 3 NO₃), and duplex (MM_{(3′1S:A, G)}).

(12) Rag1-40 was a derivative of Rag1-26, wherein an asymmetric structure with a blunt end was introduced by removing the overhang of the sense strand. The structural combination formula is: sense strand (S₁₉), antisense strand (AS₁₉+3NO₂), and duplex (B3+MM_{(3′1S:A, C)}).

(13) Rag1-41 was similar to Rag1-40, except that the mispaired cytosine nucleotide of the sense strand was replaced by a guanine nucleotide. The structural combination formula is: sense strand (S₁₉), antisense strand (AS₁₉+3NO₂), and duplex (B3+MM_{(3′1S:A, G)}).

(14) Rag1-42 was also similar to Rag1-40, except that the mispaired nucleotide was deleted from the sense strand, leading to a single-nucleotide overhang at the 5′ terminus of the antisense strand. The structural combination formula is: sense strand (S₁₈), antisense strand (5NO₁+AS₁₈+3NO₂), and duplex (OH_{AS5′}).

(15) Rag1-43 was also similar to Rag1-40, except that the duplex length was reduced by one base pair. The structural combination formula is: sense strand (S₁₈), antisense strand (AS₁₈+3NO₂), and duplex (B3+MM_{(3′1S:A, C)}).

(16) Rag1-44 was similar to Rag1-37, except that the duplex length was reduced by one base pair. The structural combination formula is: sense strand (S₁₇), antisense strand (5NO₁+AS₁₈+3NO₃), and duplex (OH_{AS5′}).

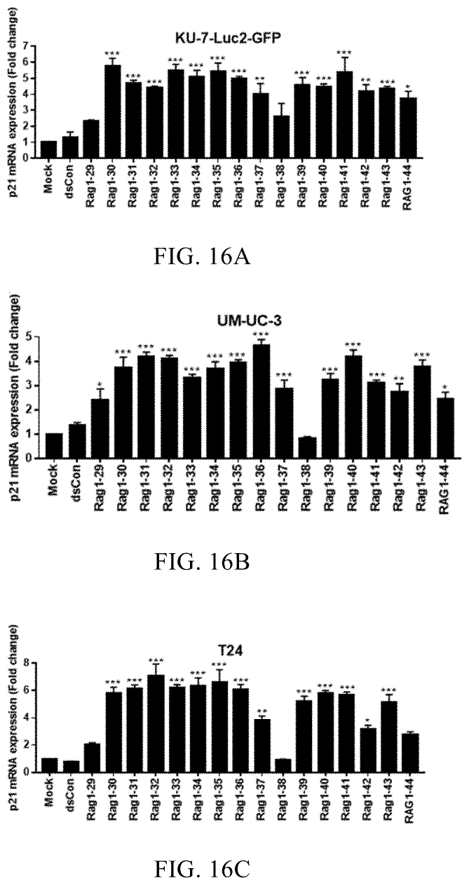

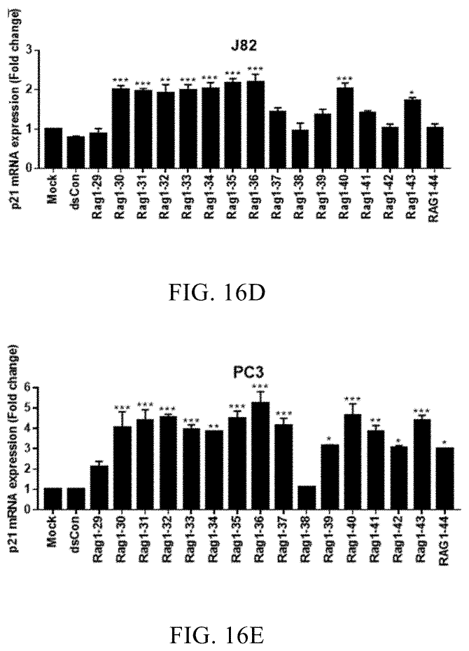

The aforementioned duplex saRNAs (Rag1-29 to Rag1-44) were transfected into Ku-7-luc2-GFP, UM-UC-3, T24, J82, or PC3 cell lines, respectively, and 72 hours after transfection, p21 mRNA expression was evaluated. As shown in FIG. 16A-16E, Rag1-29 and Rag1-38 almost completely lost their activity in inducing p21 expression in all cell lines. These two duplexes did not have an overhang of at least two nucleotides on their antisense strands, indicating this feature is indispensable for p21 expression induction. In contrast, a 3′ overhang on the sense strand was totally dispensable, since Rag1-31, Rag1-35, Rag1-36, Rag1-40, Rag1-41, and Rag1-43 possessed strong activities. In addition, the presence of the nucleotide mispairing at the 3′ terminus of a duplex was critical for maximizing activity, because the removal of the mispaired nucleotide at the 3′ terminus of the sense strand of Rag1-37, Rag1-42, and Rag1-44 led to a decrease in p21 induction activity compared to other strong activators. Replacement of the cytosine mismatch on the sense strand by a guanine to form a G-U mispairing with the uracil on the antisense strand (for example, in Rag1-39 and Rag1-41) had an adverse impact on the activity of the saRNA with the exception for Rag1-33. A plausible explanation is that a positive influence resulted from the longer length of its duplex region (20 nucleotides, compared with 18 and 19 nucleotides of Rag1-39 and Rag1-40, respectively) offset the negative impact of the G-U mispairing. Nonetheless, this further supports a preference for cytosine mispairing over other nucleotides for enhanced gene induction activity of a saRNA.

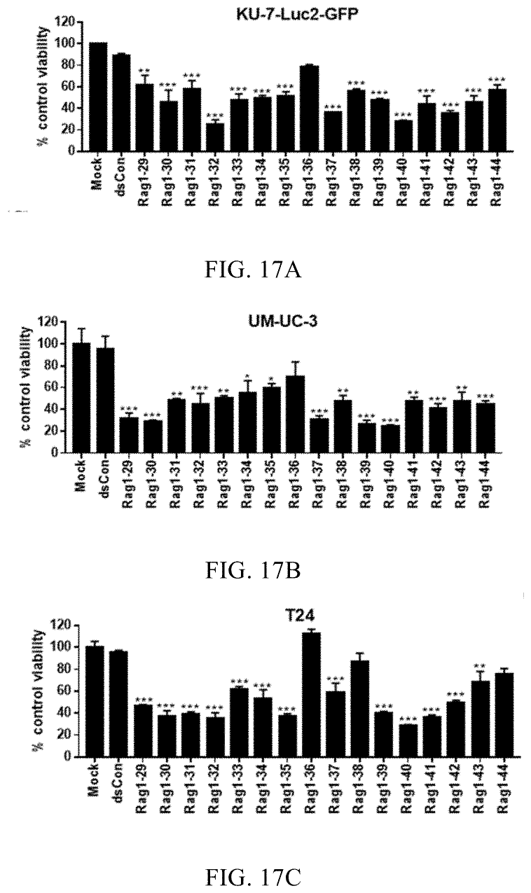

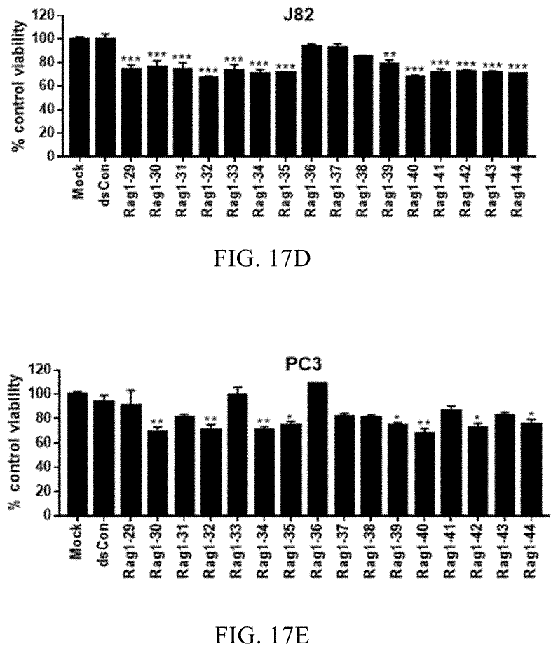

The cell viability of each cell line (FIG. 17A-17E) was assayed by the CCK-8 method. It was discovered by a comprehensive analysis in combination with the gene-inducing activity (FIG. 16A-16E) that duplexes Rag1-30, Rag1-32, Rag1-35, and Rag1-40 all had a maximum gene-inducing activity and a strong suppressing effect against the proliferation of the cancer cells. Although Rag1-35 and Rag1-40 were variants of Rag1-23 and Rag1-26, respectively, and both have an asymmetric structure with a blunt end, a cytosine mispairing, and a natural nucleotide overhang, Rag1-40 showed significantly improved activity in gene induction and tumor cell growth suppression due to 100% complementarity of the full length of the antisense strand of its duplex with its target.

The duplex of Rag1-36 was a potent activator for p21 expression, but lost part or all of the cell proliferation-suppressing activity in the tested cell lines. It was the only duplex whose gene-inducing activity did not correlate to its cell proliferation-suppressing activity among all tested saRNA variants. This unique property could be related to the sequence and/or structural feature of Rag1-36 and have utility in a situation where only the induction of the target gene expression, but not inhibition of cell proliferation, is desired.

### Example 9: Validation of Optimized Combinations of Structural Features

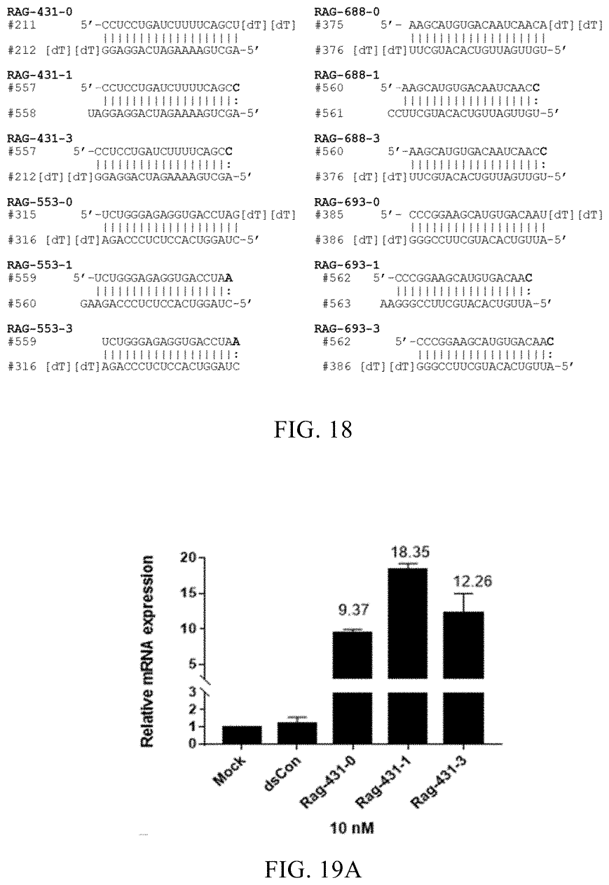

saRNAs of conventional structures (i.e. RAG-431-0, RAG-553-0, RAG-688-0 and RAG-693-0) targeting the p21 gene promoter and three types of variants thereof were designed and synthesized. Their structural combination formulas are as follows (FIG. 18):

RAG-431-1: sense strand (S₁₉), antisense strand (AS₁₉+3NO₂), and duplex (B3+MM_{(3′1S:A, C)})

RAG-431-3: sense strand (S₁₉+3T₂), antisense strand (AS₁₉), and duplex (B5+MM_{(3′1S:A, C)})

RAG-553-1: sense strand (S₁₉), antisense strand (AS₁₉+3NO₂), and duplex (B3+MM_{(3′1S:A, C)})

RAG-553-3: sense strand (S₁₉+3T₂), antisense strand (AS₁₉), and duplex (B5+MM_{(3′1S:A, C)})

RAG-688-1: sense strand (S₁₉), antisense strand (AS₁₉+3NO₂), and duplex (B3+MM_{(3′1S:A, C)})

RAG-688-3: sense strand (S₁₉+3T₂), antisense strand (AS₁₉), and duplex (B5+MM_{(3′1S:A, C)})

RAG-693-1: sense strand (S₁₉), antisense strand (AS₁₉+3NO₂), and duplex (B3+MM_{(3′1S:A, C)}))

RAG-693-3: sense strand (S₁₉+3T₂), antisense strand (AS₁₉), and duplex (B5+MM_{(5′1AS: U,C)})

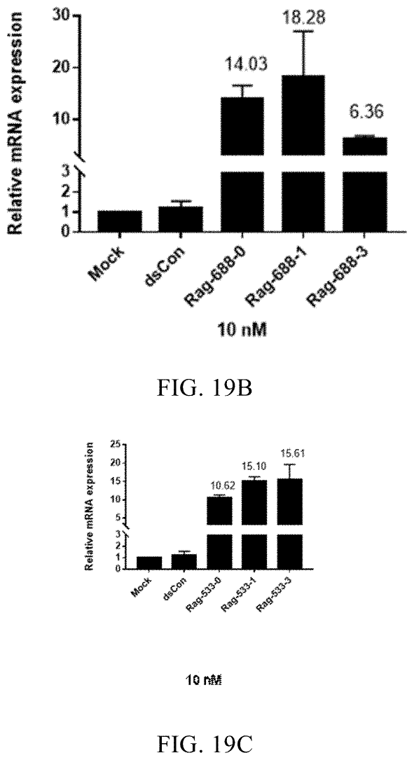

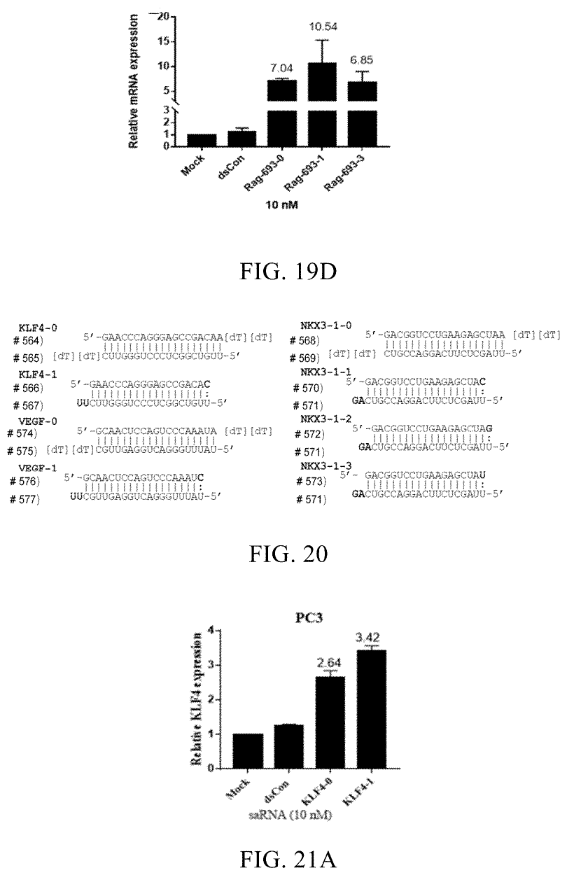

RAG-431-0 and the variants thereof were transfected into Ku-7-luc2-GFP cells, wherein RAG-431-0 induced a 9.37-fold change in p21 expression, and its variants of RAG-431-1 and RAG-431-3 induced an 18.35-fold change and a 12.26-fold change in p21 expression, respectively (FIG. 19).

RAG-553-0 and the variants thereof were transfected into Ku-7-luc2-GFP cells, wherein RAG-553-0 induced a 10.62-fold change in p21 expression, and its variants of RAG-553-1 and RAG-553-3 induced a 15.10-fold change and a 15.61-fold change in p21 expression, respectively (FIG. 19).

RAG-688-0 and the variants thereof were transfected into Ku-7-luc2-GFP cells, wherein RAG-688-0 induced a 14.03-fold change in p21 expression, and its variants of RAG-688-1 and RAG-688-3 induced an 18.28-fold change and a 6.36-fold change in p21 expression, respectively (FIG. 19).

RAG-693-0 and the variants thereof were transfected into Ku-7-luc2-GFP cells, wherein RAG-693-0 induced a 7.04-fold change in p21 expression, and its variants of RAG-693-1 and RAG-693-3 induced a 10.54-fold change and a 6.85-fold change in p21 expression, respectively (FIG. 19).

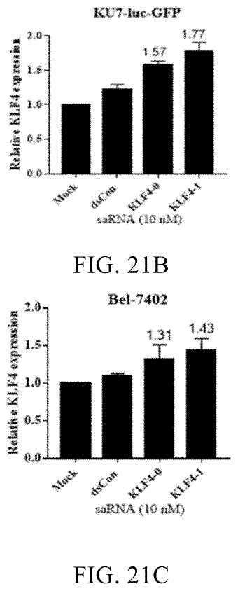

saRNAs (KLF4-0) targeting the KLF4 gene promoter and its variant KLF4-1 with structures similar to that of Rag1-40, were also designed and synthesized, and the structural combination formula for KLF4-1 is: sense strand (S₁₉), antisense strand (AS₁₉+3 NO₂), and duplex (B3+MM_{(3′1S:A, C)}) (FIG. 20). These saRNAs were transfected into PC3, Ku-7-luc2-GFP, and Bel-7402 cells, wherein KLF4-0 induced a 2.64-, 1.57-, and 1.31-fold change in KLF4 expression, respectively in those cells, while the variant KLF4-1 exhibited a higher activity in inducing KLF4 expression than did KLF4-0, and induced a 3.42-, 1.77-, and 1.43-fold change, respectively, in the PC3, Ku-7-luc2-GFP, and Bel-7402 cells (FIG. 21A-21C).

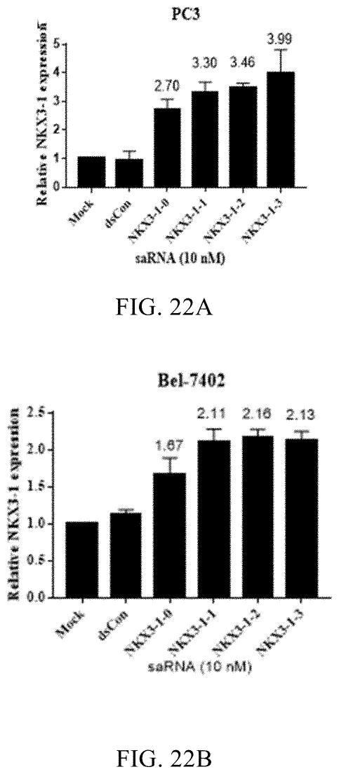

saRNAs (NKX3-1-0) targeting the NKX3-1 gene promoter and its variants NKX3-1-1, NKX3-1-2, and NKX3-1-3 were also designed and synthesized. The structures of these variants were similar to that of Rag1-40. The structural combination formula of NKX3-1-1 is: sense strand (S₁₉), antisense strand (AS₁₉ 3 NO₂) and duplex (B3+MM_{(3′1S:A, C)}); the structural combination formula of NKX3-1-2 is: sense strand (S₁₉), antisense strand (AS₁₉+3NO₂), and duplex (B3+MM_{(3′1S:A, C)}); and the structural combination formula of NKX3-1-3 is: sense strand (S₁₉), antisense strand (AS₁₉ 3 NO₂), and duplex (B3+MM_{(3′1S:A, C)}) (FIG. 20). These saRNAs were transfected into PC3 and Bel-7402 cells, wherein NKX3-1-0 induced a 2.7- and 1.67-fold change in NKX3-1 expression respectively in the two cell types, while the activities of all the variants (NKX3-1-1, NKX3-1-2, and NKX3-1-3) in inducing NKX3-1 expression were higher than that of NKX3-1-0 (FIG. 22A-22B).

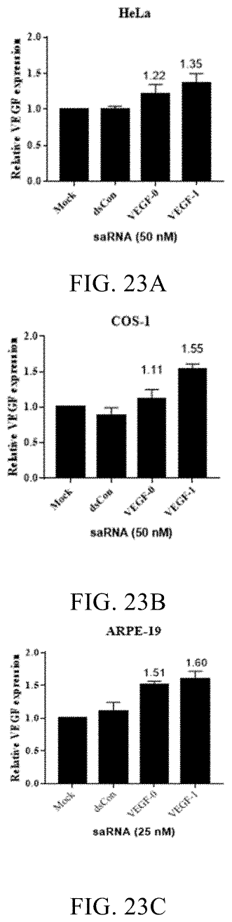

saRNAs (VEGF-0) targeting the VEGFA gene promoter and its variant VEGF-1 with structures similar to that of Rag1-40, were also designed and synthesized, and the structural combination formula of VEGF-1 is: sense strand (S₁₉), antisense strand (AS₁₉+3NO₂), and duplex (B3+MM_{(3′1S:A, C)}) (FIG. 20). These saRNAs were transfected into HeLa, COS-1, or ARPE-19 cells, wherein VEGF-0 induced a 1.22-, 1.11-, and 1.51-fold change in VEGFA expression, respectively in the 3 types of cells, while the activity of the variant VEGF-1 in inducing VEGFA expression was higher than that of VEGF-0 (FIG. 23A-23C).

From the above analysis, it was concluded that saRNAs with a structure similar to Rag1-40 were superior than saRNAs with conventional structure in terms of gene-activating activity and the enhancement was independent of the sequence of the saRNAs. Specifically, the 3′ overhang of the antisense strand could be a natural nucleotide overhang (such as RAG-431-1, RAG-553-1, RAG-688-1, RAG-693-1, NKX3-1-1, NKX3-1-2, and NKX3-1-3) or a UU overhang (such as KLF4-1 and VEGF-1). The mutated nucleotides of the sense strand can be C (such as RAG-431-1, RAG-431-3, RAG-688-1, RAG-688-3, RAG-693-1, NKX3-1-1, KLF4-1, VEGF-1), A (RAG-553-1 and RAG-553-3), G (NKX3-1-2), and U (such as NKX3-1-3).

### Example 10: Inhibition of the Growth of Subcutaneously Human Xenograft Tumors in Mice by p21 saRNA Rag1-40

Formulation of saRNA: In vivo-jetPEI (201-10G, Polyplus-transfection, France) was used to deliver saRNA, and the saRNA was formulated according to the preparation method provided by the manufacturer. Briefly, the saRNA was first diluted in 10% glucose solution to obtain solution A; a required amount of in vivo-jetPEI was diluted in the 10% glucose solution to obtain solution B; then equal volumes of solution A and solution B were combined and mixed well to give a formulation with a nitrogen-to-phosphorus (N:P) ratio of 8 and a final concentration of glucose at 5%. The mixture was incubated at room temperature for 15 minutes before injection into mice.

HepG2 human hepatocellular carcinoma cells at the logarithmic phase were harvested and counted to make a suspension of 3.5×10⁷ cells per milliliter (mL). The cell suspension (0.2 mL/mouse) was then subcutaneously inoculated into the right armpit of BALB/c nude mice. When tumors grew to about 100 mm³, the tumor-bearing mice were randomly divided into two groups: a Vehicle (5% glucose) group and a Rag1-40-treated group, with 7 mice per group, and the saRNA formulation or vehicle control was injected into the tumors at a dose of 1 mg·kg-1 saRNA on day 1, day 4, day 7 and day 10. The long and short diameters of the tumors were measured every two days with a Vernier caliper starting from the first administration (day 1), and the volumes of the tumors were calculated according to a formula: V=(L×W²)/2, wherein L represents the longest diameter of a tumor, and W represents a diameter parallel with the tumor surface and perpendicular to the long diameter. A tumor growth curve during administration, as well as weight and morphology of the tumors after harvest, were recorded.

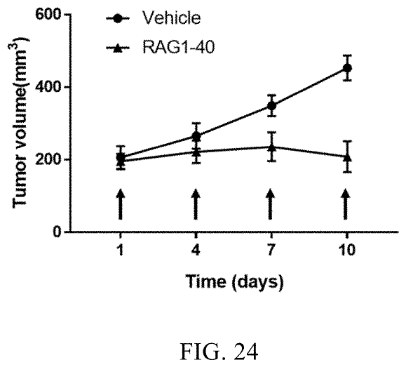

As shown in FIG. 24, Rag1-40 treated tumors exhibited a slower growth from day 4 than vehicle treated tumors and appeared to shrink from day 7 of treatment. On day 10 of treatment, the mean volume of Rag1-40 treated tumors increased by 12.8% compared to the volume at the beginning of treatment, whereas the mean volume of the tumors in the Vehicle group increased by 119.8%. The difference in the final tumor volume between the two groups was statistically significant (P<0.001), indicating that the saRNA capable of activating p21 expression had a significant in vivo antitumor effect.

### INCORPORATION BY REFERENCE

All disclosures of each patent literature and scientific literature cited herein are incorporated herein by reference for all purposes.

### EQUIVALENCE

The present invention can be implemented in other specific forms without departing from its fundamental characteristics. Therefore, the aforementioned examples shall be considered as illustrative rather than restrictive to the present invention described herein. The scope of the present invention is represented by the appended claims rather than the above specification and is intended to cover all changes falling into the meanings and scopes of equivalents of the claims.

1. A small activating RNA, wherein the small activating RNA is composed of:
i. a first oligonucleotide strand containing 17 to 30 nucleotides; and
ii. a second oligonucleotide strand containing 17 to 30 nucleotides, wherein a sequence of at least 15 nucleotides in length in the first oligonucleotide strand is complementary to the second oligonucleotide strand to form a duplex, and wherein the first oligonucleotide strand or the second oligonucleotide strand has more than 75% homology or complementarity with any continuous fragment of 15 to 30 nucleotides in length in the promoter of a target gene;
wherein one end of the duplex is a blunt end, and the other end of the duplex has an overhang with 1 to 4 nucleotides at the terminus of the first oligonucleotide strand or the second oligonucleotide strand.

2. The small activating RNA of claim 1, wherein one end of the duplex is a blunt end, and the other end has an overhang with 2 or 3 nucleotides at the terminus of the first oligonucleotide strand or the second oligonucleotide strand.

3. The small activating RNA of claim 1, wherein the nucleotides of the overhang are selected from thymine, uracil, or natural nucleotides.

4. The small activating RNA of claim 3, wherein the overhang is selected from dTdTdT, dTdT, UUU, UU, or 2 or 3 continuous natural nucleotides.

5. The small activating RNA of claim 1, wherein the blunt end of the duplex is at a 5′ terminus of the first or second oligonucleotide strand, wherein 1 to 3 nucleotides of the first to third nucleotides from the 5′ terminus in the first or second oligonucleotide strand are mispaired with nucleotides at the corresponding positions in the other strand.

6. The small activating RNA of claim 5, wherein the mispaired nucleotide is a cytosine.

7. The small activating RNA of claim 1, wherein the length of the duplex formed by the first oligonucleotide strand and the second oligonucleotide strand is 17 to 24 nucleotides.

8. The small activating RNA of claim 7, wherein the length of the duplex formed by the first oligonucleotide strand and the second oligonucleotide strand is 18 to 20 nucleotides.

9. The use of the small activating RNA of claim 1 in the preparation of a formulation for activating or upregulating the expression of a target gene in a cell.

10. The use of claim 9, wherein the small activating RNA is introduced into the cell directly.

11. The use of claim 10, wherein the cell is a mammalian cell.

12. The use of claim 11, wherein the cell is a human cell and is present in a human body.

13. The use of claim 12, wherein the human body suffers from a disease caused by the defect and/or deficiency of target gene expression, and the small activating RNA is administrated in an effective amount to treat the disease

14. The small activating RNA of claim 1, wherein the target gene is selected from the group consisting of human p21, KLF4, NKX3-1, and VEGFA.

15. The small activating RNA of claim 14, wherein the small activating RNA activates or upregulates the expression of p21 by at least 10%.

16. A composition, comprising the small activating RNA of claim 14 and a pharmaceutically acceptable carrier.

17. The composition of claim 16, wherein the pharmaceutically acceptable carrier is a liposome, a macromolecular polymer, or a polypeptide.

18. The use of the small activating RNA of claim 14 in the preparation of a formulation for activating or upregulating the expression of the target gene.

19. The use of claim 18, wherein the preparation of the formulation is for treating a tumor or a benign proliferative lesion.

20. The use of claim 19, wherein the tumor is selected from a bladder cancer, a prostate cancer, a hepatoma, and a colorectal cancer.

21. A method of treating a bladder cancer, a prostate cancer, a hepatoma, or a colorectal cancer in a subject in need thereof, the method comprising administering to the subject in need thereof an effective amount of the composition of claim 16.


SEQUENCE LISTING (601 sequences)

SEQ ID NO: 1 - DNA, 21 bp, artificial sequence
  Small Activating RNA
acuacugagugacaguagatt

SEQ ID NO: 2 - DNA, 21 bp, artificial sequence
  Small Activating RNA
ucuacugucacucaguagutt

SEQ ID NO: 3 - DNA, 21 bp, artificial sequence
  Small Activating RNA
cuggagagugccaacucautt

SEQ ID NO: 4 - DNA, 21 bp, artificial sequence
  Small Activating RNA
augaguuggcacucuccagtt

SEQ ID NO: 5 - DNA, 21 bp, artificial sequence
  Small Activating RNA
uggagagugccaacucauutt

SEQ ID NO: 6 - DNA, 21 bp, artificial sequence
  Small Activating RNA
aaugaguuggcacucuccatt

SEQ ID NO: 7 - DNA, 21 bp, artificial sequence
  Small Activating RNA
ggagagugccaacucauuctt

SEQ ID NO: 8 - DNA, 21 bp, artificial sequence
  Small Activating RNA
gaaugaguuggcacucucctt

SEQ ID NO: 9 - DNA, 21 bp, artificial sequence
  Small Activating RNA
gagagugccaacucauucutt

SEQ ID NO: 10 - DNA, 21 bp, artificial sequence
  Small Activating RNA
agaaugaguuggcacucuctt

SEQ ID NO: 11 - DNA, 21 bp, artificial sequence
  Small Activating RNA
agagugccaacucauucuctt

SEQ ID NO: 12 - DNA, 21 bp, artificial sequence
  Small Activating RNA
gagaaugaguuggcacucutt

SEQ ID NO: 13 - DNA, 21 bp, artificial sequence
  Small Activating RNA
gagugccaacucauucucctt

SEQ ID NO: 14 - DNA, 21 bp, artificial sequence
  Small Activating RNA
ggagaaugaguuggcacuctt

SEQ ID NO: 15 - DNA, 21 bp, artificial sequence
  Small Activating RNA
agugccaacucauucuccatt

SEQ ID NO: 16 - DNA, 21 bp, artificial sequence
  Small Activating RNA
uggagaaugaguuggcacutt

SEQ ID NO: 17 - DNA, 21 bp, artificial sequence
  Small Activating RNA
gugccaacucauucuccaatt

SEQ ID NO: 18 - DNA, 21 bp, artificial sequence
  Small Activating RNA
uuggagaaugaguuggcactt

SEQ ID NO: 19 - DNA, 21 bp, artificial sequence
  Small Activating RNA
ugccaacucauucuccaagtt

SEQ ID NO: 20 - DNA, 21 bp, artificial sequence
  Small Activating RNA
cuuggagaaugaguuggcatt

SEQ ID NO: 21 - DNA, 21 bp, artificial sequence
  Small Activating RNA
gccaacucauucuccaagutt

SEQ ID NO: 22 - DNA, 21 bp, artificial sequence
  Small Activating RNA
acuuggagaaugaguuggctt

SEQ ID NO: 23 - DNA, 21 bp, artificial sequence
  Small Activating RNA
ccaacucauucuccaaguatt

SEQ ID NO: 24 - DNA, 21 bp, artificial sequence
  Small Activating RNA
uacuuggagaaugaguuggtt

SEQ ID NO: 25 - DNA, 21 bp, artificial sequence
  Small Activating RNA
caacucauucuccaaguaatt

SEQ ID NO: 26 - DNA, 21 bp, artificial sequence
  Small Activating RNA
uuacuuggagaaugaguugtt

SEQ ID NO: 27 - DNA, 21 bp, artificial sequence
  Small Activating RNA
aaaagccagauuuguggcutt

SEQ ID NO: 28 - DNA, 21 bp, artificial sequence
  Small Activating RNA
agccacaaaucuggcuuuutt

SEQ ID NO: 29 - DNA, 21 bp, artificial sequence
  Small Activating RNA
aaagccagauuuguggcuctt

SEQ ID NO: 30 - DNA, 21 bp, artificial sequence
  Small Activating RNA
gagccacaaaucuggcuuutt

SEQ ID NO: 31 - DNA, 21 bp, artificial sequence
  Small Activating RNA
aagccagauuuguggcucatt

SEQ ID NO: 32 - DNA, 21 bp, artificial sequence
  Small Activating RNA
ugagccacaaaucuggcuutt

SEQ ID NO: 33 - DNA, 21 bp, artificial sequence
  Small Activating RNA
agccagauuuguggcucactt

SEQ ID NO: 34 - DNA, 21 bp, artificial sequence
  Small Activating RNA
gugagccacaaaucuggcutt

SEQ ID NO: 35 - DNA, 21 bp, artificial sequence
  Small Activating RNA
gccagauuuguggcucacutt

SEQ ID NO: 36 - DNA, 21 bp, artificial sequence
  Small Activating RNA
agugagccacaaaucuggctt

SEQ ID NO: 37 - DNA, 21 bp, artificial sequence
  Small Activating RNA
ccaacucauucuccaaguaaa

SEQ ID NO: 38 - DNA, 21 bp, artificial sequence
  Small Activating RNA
uacuuggagaaugaguuggca

SEQ ID NO: 39 - DNA, 21 bp, artificial sequence
  Small Activating RNA
ccaacucauucuccaaguauu

SEQ ID NO: 40 - DNA, 21 bp, artificial sequence
  Small Activating RNA
uacuuggagaaugaguugguu

SEQ ID NO: 41 - DNA, 22 bp, artificial sequence
  Small Activating RNA
ccaacucauucuccaaguaatt

SEQ ID NO: 42 - DNA, 22 bp, artificial sequence
  Small Activating RNA
uuacuuggagaaugaguuggtt

SEQ ID NO: 43 - DNA, 23 bp, artificial sequence
  Small Activating RNA
ccaacucauucuccaaguaaatt

SEQ ID NO: 44 - DNA, 23 bp, artificial sequence
  Small Activating RNA
uuuacuuggagaaugaguuggtt

SEQ ID NO: 45 - DNA, 24 bp, artificial sequence
  Small Activating RNA
ccaacucauucuccaaguaaaatt

SEQ ID NO: 46 - DNA, 24 bp, artificial sequence
  Small Activating RNA
uuuuacuuggagaaugaguuggtt

SEQ ID NO: 47 - DNA, 25 bp, artificial sequence
  Small Activating RNA
ccaacucauucuccaaguaaaaatt

SEQ ID NO: 48 - DNA, 25 bp, artificial sequence
  Small Activating RNA
uuuuuacuuggagaaugaguuggtt

SEQ ID NO: 49 - DNA, 26 bp, artificial sequence
  Small Activating RNA
ccaacucauucuccaaguaaaaaatt

SEQ ID NO: 50 - DNA, 26 bp, artificial sequence
  Small Activating RNA
uuuuuuacuuggagaaugaguuggtt

SEQ ID NO: 51 - DNA, 21 bp, artificial sequence
  Small Activating RNA
gccaacucauucuccaaguaa

SEQ ID NO: 52 - DNA, 21 bp, artificial sequence
  Small Activating RNA
acuuggagaaugaguuggcac

SEQ ID NO: 53 - DNA, 22 bp, artificial sequence
  Small Activating RNA
gccaacucauucuccaaguatt

SEQ ID NO: 54 - DNA, 22 bp, artificial sequence
  Small Activating RNA
uacuuggagaaugaguuggctt

SEQ ID NO: 55 - DNA, 23 bp, artificial sequence
  Small Activating RNA
gccaacucauucuccaaguaatt

SEQ ID NO: 56 - DNA, 23 bp, artificial sequence
  Small Activating RNA
uuacuuggagaaugaguuggctt

SEQ ID NO: 57 - DNA, 24 bp, artificial sequence
  Small Activating RNA
gccaacucauucuccaaguaaatt

SEQ ID NO: 58 - DNA, 24 bp, artificial sequence
  Small Activating RNA
uuuacuuggagaaugaguuggctt

SEQ ID NO: 59 - DNA, 25 bp, artificial sequence
  Small Activating RNA
gccaacucauucuccaaguaaaatt

SEQ ID NO: 60 - DNA, 25 bp, artificial sequence
  Small Activating RNA
uuuuacuuggagaaugaguuggctt

SEQ ID NO: 61 - DNA, 26 bp, artificial sequence
  Small Activating RNA
gccaacucauucuccaaguaaaaatt

SEQ ID NO: 62 - DNA, 26 bp, artificial sequence
  Small Activating RNA
uuuuuacuuggagaaugaguuggctt

SEQ ID NO: 63 - DNA, 21 bp, artificial sequence
  Small Activating RNA
ccaacucauucuccaaguutt

SEQ ID NO: 64 - DNA, 21 bp, artificial sequence
  Small Activating RNA
ccaacucauucuccaaguctt

SEQ ID NO: 65 - DNA, 21 bp, artificial sequence
  Small Activating RNA
ccaacucauucuccaagautt

SEQ ID NO: 66 - DNA, 19 bp, artificial sequence
  Small Activating RNA
ccaacucauucuccaagua

SEQ ID NO: 67 - DNA, 18 bp, artificial sequence
  Small Activating RNA
caacucauucuccaagua

SEQ ID NO: 68 - DNA, 20 bp, artificial sequence
  Small Activating RNA
caacucauucuccaaguatt

SEQ ID NO: 69 - DNA, 22 bp, artificial sequence
  Small Activating RNA
gccaacucauucuccaaguauu

SEQ ID NO: 70 - DNA, 22 bp, artificial sequence
  Small Activating RNA
uacuuggagaaugaguuggcuu

SEQ ID NO: 71 - DNA, 22 bp, artificial sequence
  Small Activating RNA
gccaacucauucuccaaguaaa

SEQ ID NO: 72 - DNA, 22 bp, artificial sequence
  Small Activating RNA
uacuuggagaaugaguuggcac

SEQ ID NO: 73 - DNA, 22 bp, artificial sequence
  Small Activating RNA
gccaacucauucuccaagucuu

SEQ ID NO: 74 - DNA, 20 bp, artificial sequence
  Small Activating RNA
caacucauucuccaagucuu

SEQ ID NO: 75 - DNA, 21 bp, artificial sequence
  Small Activating RNA
ccaacucauucuccaagucuu

SEQ ID NO: 76 - DNA, 21 bp, artificial sequence
  Small Activating RNA
ccaacucauucuccaagucaa

SEQ ID NO: 77 - DNA, 20 bp, artificial sequence
  Small Activating RNA
caacucauucuccaagucaa

SEQ ID NO: 78 - DNA, 22 bp, artificial sequence
  Small Activating RNA
gccaacucauucuccaagucaa

SEQ ID NO: 79 - DNA, 20 bp, artificial sequence
  Small Activating RNA
uacuuggagaaugaguuggc

SEQ ID NO: 80 - DNA, 18 bp, artificial sequence
  Small Activating RNA
caacucauucuccaaguc

SEQ ID NO: 81 - DNA, 22 bp, artificial sequence
  Small Activating RNA
gccaacucauucuccaagugaa

SEQ ID NO: 82 - DNA, 20 bp, artificial sequence
  Small Activating RNA
gccaacucauucuccaaguc

SEQ ID NO: 83 - DNA, 19 bp, artificial sequence
  Small Activating RNA
ccaacucauucuccaaguc

SEQ ID NO: 84 - DNA, 18 bp, artificial sequence
  Small Activating RNA
ccaacucauucuccaagu

SEQ ID NO: 85 - DNA, 21 bp, artificial sequence
  Small Activating RNA
uuuacuuggagaaugaguugg

SEQ ID NO: 86 - DNA, 20 bp, artificial sequence
  Small Activating RNA
caacucauucuccaagugaa

SEQ ID NO: 87 - DNA, 19 bp, artificial sequence
  Small Activating RNA
ccaacucauucuccaagug

SEQ ID NO: 88 - DNA, 17 bp, artificial sequence
  Small Activating RNA
caacucauucuccaagu

SEQ ID NO: 89 - DNA, 21 bp, artificial sequence
  Synthesized primer for PCR
atcaccatcttccaggagcga

SEQ ID NO: 90 - DNA, 21 bp, artificial sequence
  Synthesized primer for PCR
ttctccatggtggtgaagacg

SEQ ID NO: 91 - DNA, 20 bp, artificial sequence
  Synthesized primer for PCR
ggaagaccatgtggacctgt

SEQ ID NO: 92 - DNA, 20 bp, artificial sequence
  Synthesized primer for PCR
ggattagggcttcctcttgg

SEQ ID NO: 93 - DNA, 92 bp, Homo sapiens
ggctatgtggggagtattcaggagacagacaactcactcgtcaaatcctccccttcctggccaacaaagctgctgcaaccacagggatttct

SEQ ID NO: 94 - DNA, 86 bp, Homo sapiens
ggtagtctctccaattccctccttcccggaagcatgtgacaatcaacaactttgtatacttaagttcagtggacctcaatttcctc

SEQ ID NO: 95 - DNA, 35 bp, Homo sapiens
ctttgttggggtgtctaggtgctccaggtgcttct

SEQ ID NO: 96 - DNA, 51 bp, Homo sapiens
ttctgggagaggtgacctagtgagggatcagtgggaatagaggtgatattg

SEQ ID NO: 97 - DNA, 30 bp, Homo sapiens
aggtgatattgtggggcttttctggaaatt

SEQ ID NO: 98 - DNA, 38 bp, Homo sapiens
attaatgtcatcctcctgatcttttcagctgcattggg

SEQ ID NO: 99 - DNA, 40 bp, Homo sapiens
tctaacagtgctgtgtcctcctggagagtgccaactcatt

SEQ ID NO: 100 - DNA, 66 bp, Homo sapiens
gtgccaactcattctccaagtaaaaaaagccagatttgtggctcacttcgtggggaaatgtgtcca

SEQ ID NO: 101 - DNA, 21 bp, artificial sequence
  Small Activating RNA
ucguggggaaauguguccatt

SEQ ID NO: 102 - DNA, 21 bp, artificial sequence
  Small Activating RNA
uggacacauuuccccacgatt

SEQ ID NO: 103 - DNA, 21 bp, artificial sequence
  Small Activating RNA
uucguggggaaaugugucctt

SEQ ID NO: 104 - DNA, 21 bp, artificial sequence
  Small Activating RNA
ggacacauuuccccacgaatt

SEQ ID NO: 105 - DNA, 21 bp, artificial sequence
  Small Activating RNA
cuucguggggaaauguguctt

SEQ ID NO: 106 - DNA, 21 bp, artificial sequence
  Small Activating RNA
gacacauuuccccacgaagtt

SEQ ID NO: 107 - DNA, 21 bp, artificial sequence
  Small Activating RNA
acuucguggggaaaugugutt

SEQ ID NO: 108 - DNA, 21 bp, artificial sequence
  Small Activating RNA
acacauuuccccacgaagutt

SEQ ID NO: 109 - DNA, 21 bp, artificial sequence
  Small Activating RNA
cacuucguggggaaaugugtt

SEQ ID NO: 110 - DNA, 21 bp, artificial sequence
  Small Activating RNA
cacauuuccccacgaagugtt

SEQ ID NO: 111 - DNA, 21 bp, artificial sequence
  Small Activating RNA
ucacuucguggggaaaugutt

SEQ ID NO: 112 - DNA, 21 bp, artificial sequence
  Small Activating RNA
acauuuccccacgaagugatt

SEQ ID NO: 113 - DNA, 21 bp, artificial sequence
  Small Activating RNA
cucacuucguggggaaaugtt

SEQ ID NO: 114 - DNA, 21 bp, artificial sequence
  Small Activating RNA
cauuuccccacgaagugagtt

SEQ ID NO: 115 - DNA, 21 bp, artificial sequence
  Small Activating RNA
gcucacuucguggggaaautt

SEQ ID NO: 116 - DNA, 21 bp, artificial sequence
  Small Activating RNA
auuuccccacgaagugagctt

SEQ ID NO: 117 - DNA, 21 bp, artificial sequence
  Small Activating RNA
ggcucacuucguggggaaatt

SEQ ID NO: 118 - DNA, 21 bp, artificial sequence
  Small Activating RNA
uuuccccacgaagugagcctt

SEQ ID NO: 119 - DNA, 21 bp, artificial sequence
  Small Activating RNA
uggcucacuucguggggaatt

SEQ ID NO: 120 - DNA, 21 bp, artificial sequence
  Small Activating RNA
uuccccacgaagugagccatt

SEQ ID NO: 121 - DNA, 21 bp, artificial sequence
  Small Activating RNA
guggcucacuucguggggatt

SEQ ID NO: 122 - DNA, 21 bp, artificial sequence
  Small Activating RNA
uccccacgaagugagccactt

SEQ ID NO: 123 - DNA, 21 bp, artificial sequence
  Small Activating RNA
uguggcucacuucguggggtt

SEQ ID NO: 124 - DNA, 21 bp, artificial sequence
  Small Activating RNA
ccccacgaagugagccacatt

SEQ ID NO: 125 - DNA, 21 bp, artificial sequence
  Small Activating RNA
uuuguggcucacuucguggtt

SEQ ID NO: 126 - DNA, 21 bp, artificial sequence
  Small Activating RNA
ccacgaagugagccacaaatt

SEQ ID NO: 127 - DNA, 21 bp, artificial sequence
  Small Activating RNA
auuuguggcucacuucgugtt

SEQ ID NO: 128 - DNA, 21 bp, artificial sequence
  Small Activating RNA
cacgaagugagccacaaautt

SEQ ID NO: 129 - DNA, 21 bp, artificial sequence
  Small Activating RNA
gauuuguggcucacuucgutt

SEQ ID NO: 130 - DNA, 21 bp, artificial sequence
  Small Activating RNA
acgaagugagccacaaauctt

SEQ ID NO: 131 - DNA, 21 bp, artificial sequence
  Small Activating RNA
agauuuguggcucacuucgtt

SEQ ID NO: 132 - DNA, 21 bp, artificial sequence
  Small Activating RNA
cgaagugagccacaaaucutt

SEQ ID NO: 133 - DNA, 21 bp, artificial sequence
  Small Activating RNA
cagauuuguggcucacuuctt

SEQ ID NO: 134 - DNA, 21 bp, artificial sequence
  Small Activating RNA
gaagugagccacaaaucugtt

SEQ ID NO: 135 - DNA, 21 bp, artificial sequence
  Small Activating RNA
ccagauuuguggcucacuutt

SEQ ID NO: 136 - DNA, 21 bp, artificial sequence
  Small Activating RNA
aagugagccacaaaucuggtt

SEQ ID NO: 137 - DNA, 21 bp, artificial sequence
  Small Activating RNA
gccagauuuguggcucacutt

SEQ ID NO: 138 - DNA, 21 bp, artificial sequence
  Small Activating RNA
agugagccacaaaucuggctt

SEQ ID NO: 139 - DNA, 21 bp, artificial sequence
  Small Activating RNA
agccagauuuguggcucactt

SEQ ID NO: 140 - DNA, 21 bp, artificial sequence
  Small Activating RNA
gugagccacaaaucuggcutt

SEQ ID NO: 141 - DNA, 21 bp, artificial sequence
  Small Activating RNA
aagccagauuuguggcucatt

SEQ ID NO: 142 - DNA, 21 bp, artificial sequence
  Small Activating RNA
ugagccacaaaucuggcuutt

SEQ ID NO: 143 - DNA, 21 bp, artificial sequence
  Small Activating RNA
aaagccagauuuguggcuctt

SEQ ID NO: 144 - DNA, 21 bp, artificial sequence
  Small Activating RNA
gagccacaaaucuggcuuutt

SEQ ID NO: 145 - DNA, 21 bp, artificial sequence
  Small Activating RNA
aaaagccagauuuguggcutt

SEQ ID NO: 146 - DNA, 21 bp, artificial sequence
  Small Activating RNA
agccacaaaucuggcuuuutt

SEQ ID NO: 147 - DNA, 21 bp, artificial sequence
  Small Activating RNA
caacucauucuccaaguaatt

SEQ ID NO: 148 - DNA, 21 bp, artificial sequence
  Small Activating RNA
uuacuuggagaaugaguugtt

SEQ ID NO: 149 - DNA, 21 bp, artificial sequence
  Small Activating RNA
ccaacucauucuccaaguatt

SEQ ID NO: 150 - DNA, 21 bp, artificial sequence
  Small Activating RNA
uacuuggagaaugaguuggtt

SEQ ID NO: 151 - DNA, 21 bp, artificial sequence
  Small Activating RNA
gccaacucauucuccaagutt

SEQ ID NO: 152 - DNA, 21 bp, artificial sequence
  Small Activating RNA
acuuggagaaugaguuggctt

SEQ ID NO: 153 - DNA, 21 bp, artificial sequence
  Small Activating RNA
ugccaacucauucuccaagtt

SEQ ID NO: 154 - DNA, 21 bp, artificial sequence
  Small Activating RNA
cuuggagaaugaguuggcatt

SEQ ID NO: 155 - DNA, 21 bp, artificial sequence
  Small Activating RNA
gugccaacucauucuccaatt

SEQ ID NO: 156 - DNA, 21 bp, artificial sequence
  Small Activating RNA
uuggagaaugaguuggcactt

SEQ ID NO: 157 - DNA, 21 bp, artificial sequence
  Small Activating RNA
uggagagugccaacucauutt

SEQ ID NO: 158 - DNA, 21 bp, artificial sequence
  Small Activating RNA
aaugaguuggcacucuccatt

SEQ ID NO: 159 - DNA, 21 bp, artificial sequence
  Small Activating RNA
cuggagagugccaacucautt

SEQ ID NO: 160 - DNA, 21 bp, artificial sequence
  Small Activating RNA
augaguuggcacucuccagtt

SEQ ID NO: 161 - DNA, 21 bp, artificial sequence
  Small Activating RNA
ccuggagagugccaacucatt

SEQ ID NO: 162 - DNA, 21 bp, artificial sequence
  Small Activating RNA
ugaguuggcacucuccaggtt

SEQ ID NO: 163 - DNA, 21 bp, artificial sequence
  Small Activating RNA
uccuggagagugccaacuctt

SEQ ID NO: 164 - DNA, 21 bp, artificial sequence
  Small Activating RNA
gaguuggcacucuccaggatt

SEQ ID NO: 165 - DNA, 21 bp, artificial sequence
  Small Activating RNA
cuccuggagagugccaacutt

SEQ ID NO: 166 - DNA, 21 bp, artificial sequence
  Small Activating RNA
aguuggcacucuccaggagtt

SEQ ID NO: 167 - DNA, 21 bp, artificial sequence
  Small Activating RNA
ccuccuggagagugccaactt

SEQ ID NO: 168 - DNA, 21 bp, artificial sequence
  Small Activating RNA
guuggcacucuccaggaggtt

SEQ ID NO: 169 - DNA, 21 bp, artificial sequence
  Small Activating RNA
uccuccuggagagugccaatt

SEQ ID NO: 170 - DNA, 21 bp, artificial sequence
  Small Activating RNA
uuggcacucuccaggaggatt

SEQ ID NO: 171 - DNA, 21 bp, artificial sequence
  Small Activating RNA
guccuccuggagagugccatt

SEQ ID NO: 172 - DNA, 21 bp, artificial sequence
  Small Activating RNA
uggcacucuccaggaggactt

SEQ ID NO: 173 - DNA, 21 bp, artificial sequence
  Small Activating RNA
uguguccuccuggagagugtt

SEQ ID NO: 174 - DNA, 21 bp, artificial sequence
  Small Activating RNA
cacucuccaggaggacacatt

SEQ ID NO: 175 - DNA, 21 bp, artificial sequence
  Small Activating RNA
cuguguccuccuggagagutt

SEQ ID NO: 176 - DNA, 21 bp, artificial sequence
  Small Activating RNA
acucuccaggaggacacagtt

SEQ ID NO: 177 - DNA, 21 bp, artificial sequence
  Small Activating RNA
gcuguguccuccuggagagtt

SEQ ID NO: 178 - DNA, 21 bp, artificial sequence
  Small Activating RNA
cucuccaggaggacacagctt

SEQ ID NO: 179 - DNA, 21 bp, artificial sequence
  Small Activating RNA
ugcuguguccuccuggagatt

SEQ ID NO: 180 - DNA, 21 bp, artificial sequence
  Small Activating RNA
ucuccaggaggacacagcatt

SEQ ID NO: 181 - DNA, 21 bp, artificial sequence
  Small Activating RNA
gugcuguguccuccuggagtt

SEQ ID NO: 182 - DNA, 21 bp, artificial sequence
  Small Activating RNA
cuccaggaggacacagcactt

SEQ ID NO: 183 - DNA, 21 bp, artificial sequence
  Small Activating RNA
agugcuguguccuccuggatt

SEQ ID NO: 184 - DNA, 21 bp, artificial sequence
  Small Activating RNA
uccaggaggacacagcacutt

SEQ ID NO: 185 - DNA, 21 bp, artificial sequence
  Small Activating RNA
acagugcuguguccuccugtt

SEQ ID NO: 186 - DNA, 21 bp, artificial sequence
  Small Activating RNA
caggaggacacagcacugutt

SEQ ID NO: 187 - DNA, 21 bp, artificial sequence
  Small Activating RNA
aacagugcuguguccuccutt

SEQ ID NO: 188 - DNA, 21 bp, artificial sequence
  Small Activating RNA
aggaggacacagcacuguutt

SEQ ID NO: 189 - DNA, 21 bp, artificial sequence
  Small Activating RNA
uaacagugcuguguccucctt

SEQ ID NO: 190 - DNA, 21 bp, artificial sequence
  Small Activating RNA
ggaggacacagcacuguuatt

SEQ ID NO: 191 - DNA, 21 bp, artificial sequence
  Small Activating RNA
cuaacagugcuguguccuctt

SEQ ID NO: 192 - DNA, 21 bp, artificial sequence
  Small Activating RNA
gaggacacagcacuguuagtt

SEQ ID NO: 193 - DNA, 21 bp, artificial sequence
  Small Activating RNA
ucuaacagugcuguguccutt

SEQ ID NO: 194 - DNA, 21 bp, artificial sequence
  Small Activating RNA
aggacacagcacuguuagatt

SEQ ID NO: 195 - DNA, 21 bp, artificial sequence
  Small Activating RNA
ucuuuucagcugcauugggtt

SEQ ID NO: 196 - DNA, 21 bp, artificial sequence
  Small Activating RNA
cccaaugcagcugaaaagatt

SEQ ID NO: 197 - DNA, 21 bp, artificial sequence
  Small Activating RNA
aucuuuucagcugcauuggtt

SEQ ID NO: 198 - DNA, 21 bp, artificial sequence
  Small Activating RNA
ccaaugcagcugaaaagautt

SEQ ID NO: 199 - DNA, 21 bp, artificial sequence
  Small Activating RNA
gaucuuuucagcugcauugtt

SEQ ID NO: 200 - DNA, 21 bp, artificial sequence
  Small Activating RNA
caaugcagcugaaaagauctt

SEQ ID NO: 201 - DNA, 21 bp, artificial sequence
  Small Activating RNA
ugaucuuuucagcugcauutt

SEQ ID NO: 202 - DNA, 21 bp, artificial sequence
  Small Activating RNA
aaugcagcugaaaagaucatt

SEQ ID NO: 203 - DNA, 21 bp, artificial sequence
  Small Activating RNA
cugaucuuuucagcugcautt

SEQ ID NO: 204 - DNA, 21 bp, artificial sequence
  Small Activating RNA
augcagcugaaaagaucagtt

SEQ ID NO: 205 - DNA, 21 bp, artificial sequence
  Small Activating RNA
ccugaucuuuucagcugcatt

SEQ ID NO: 206 - DNA, 21 bp, artificial sequence
  Small Activating RNA
ugcagcugaaaagaucaggtt

SEQ ID NO: 207 - DNA, 21 bp, artificial sequence
  Small Activating RNA
uccugaucuuuucagcugctt

SEQ ID NO: 208 - DNA, 21 bp, artificial sequence
  Small Activating RNA
gcagcugaaaagaucaggatt

SEQ ID NO: 209 - DNA, 21 bp, artificial sequence
  Small Activating RNA
cuccugaucuuuucagcugtt

SEQ ID NO: 210 - DNA, 21 bp, artificial sequence
  Small Activating RNA
cagcugaaaagaucaggagtt

SEQ ID NO: 211 - DNA, 21 bp, artificial sequence
  Small Activating RNA
ccuccugaucuuuucagcutt

SEQ ID NO: 212 - DNA, 21 bp, artificial sequence
  Small Activating RNA
agcugaaaagaucaggaggtt

SEQ ID NO: 213 - DNA, 21 bp, artificial sequence
  Small Activating RNA
uccuccugaucuuuucagctt

SEQ ID NO: 214 - DNA, 21 bp, artificial sequence
  Small Activating RNA
gcugaaaagaucaggaggatt

SEQ ID NO: 215 - DNA, 21 bp, artificial sequence
  Small Activating RNA
auccuccugaucuuuucagtt

SEQ ID NO: 216 - DNA, 21 bp, artificial sequence
  Small Activating RNA
cugaaaagaucaggaggautt

SEQ ID NO: 217 - DNA, 21 bp, artificial sequence
  Small Activating RNA
cauccuccugaucuuuucatt

SEQ ID NO: 218 - DNA, 21 bp, artificial sequence
  Small Activating RNA
ugaaaagaucaggaggaugtt

SEQ ID NO: 219 - DNA, 21 bp, artificial sequence
  Small Activating RNA
ucauccuccugaucuuuuctt

SEQ ID NO: 220 - DNA, 21 bp, artificial sequence
  Small Activating RNA
gaaaagaucaggaggaugatt

SEQ ID NO: 221 - DNA, 21 bp, artificial sequence
  Small Activating RNA
gucauccuccugaucuuuutt

SEQ ID NO: 222 - DNA, 21 bp, artificial sequence
  Small Activating RNA
aaaagaucaggaggaugactt

SEQ ID NO: 223 - DNA, 21 bp, artificial sequence
  Small Activating RNA
ugucauccuccugaucuuutt

SEQ ID NO: 224 - DNA, 21 bp, artificial sequence
  Small Activating RNA
aaagaucaggaggaugacatt

SEQ ID NO: 225 - DNA, 21 bp, artificial sequence
  Small Activating RNA
augucauccuccugaucuutt

SEQ ID NO: 226 - DNA, 21 bp, artificial sequence
  Small Activating RNA
aagaucaggaggaugacautt

SEQ ID NO: 227 - DNA, 21 bp, artificial sequence
  Small Activating RNA
aaugucauccuccugaucutt

SEQ ID NO: 228 - DNA, 21 bp, artificial sequence
  Small Activating RNA
agaucaggaggaugacauutt

SEQ ID NO: 229 - DNA, 21 bp, artificial sequence
  Small Activating RNA
uaaugucauccuccugauctt

SEQ ID NO: 230 - DNA, 21 bp, artificial sequence
  Small Activating RNA
gaucaggaggaugacauuatt

SEQ ID NO: 231 - DNA, 21 bp, artificial sequence
  Small Activating RNA
uuaaugucauccuccugautt

SEQ ID NO: 232 - DNA, 21 bp, artificial sequence
  Small Activating RNA
aucaggaggaugacauuaatt

SEQ ID NO: 233 - DNA, 21 bp, artificial sequence
  Small Activating RNA
auuaaugucauccuccugatt

SEQ ID NO: 234 - DNA, 21 bp, artificial sequence
  Small Activating RNA
ucaggaggaugacauuaautt

SEQ ID NO: 235 - DNA, 21 bp, artificial sequence
  Small Activating RNA
uggggcuuuucuggaaauutt

SEQ ID NO: 236 - DNA, 21 bp, artificial sequence
  Small Activating RNA
aauuuccagaaaagccccatt

SEQ ID NO: 237 - DNA, 21 bp, artificial sequence
  Small Activating RNA
guggggcuuuucuggaaautt

SEQ ID NO: 238 - DNA, 21 bp, artificial sequence
  Small Activating RNA
auuuccagaaaagccccactt

SEQ ID NO: 239 - DNA, 21 bp, artificial sequence
  Small Activating RNA
uguggggcuuuucuggaaatt

SEQ ID NO: 240 - DNA, 21 bp, artificial sequence
  Small Activating RNA
uuuccagaaaagccccacatt

SEQ ID NO: 241 - DNA, 21 bp, artificial sequence
  Small Activating RNA
uuguggggcuuuucuggaatt

SEQ ID NO: 242 - DNA, 21 bp, artificial sequence
  Small Activating RNA
uuccagaaaagccccacaatt

SEQ ID NO: 243 - DNA, 21 bp, artificial sequence
  Small Activating RNA
auuguggggcuuuucuggatt

SEQ ID NO: 244 - DNA, 21 bp, artificial sequence
  Small Activating RNA
uccagaaaagccccacaautt

SEQ ID NO: 245 - DNA, 21 bp, artificial sequence
  Small Activating RNA
uauuguggggcuuuucuggtt

SEQ ID NO: 246 - DNA, 21 bp, artificial sequence
  Small Activating RNA
ccagaaaagccccacaauatt

SEQ ID NO: 247 - DNA, 21 bp, artificial sequence
  Small Activating RNA
auauuguggggcuuuucugtt

SEQ ID NO: 248 - DNA, 21 bp, artificial sequence
  Small Activating RNA
cagaaaagccccacaauautt

SEQ ID NO: 249 - DNA, 21 bp, artificial sequence
  Small Activating RNA
gauauuguggggcuuuucutt

SEQ ID NO: 250 - DNA, 21 bp, artificial sequence
  Small Activating RNA
agaaaagccccacaauauctt

SEQ ID NO: 251 - DNA, 21 bp, artificial sequence
  Small Activating RNA
ugauauuguggggcuuuuctt

SEQ ID NO: 252 - DNA, 21 bp, artificial sequence
  Small Activating RNA
gaaaagccccacaauaucatt

SEQ ID NO: 253 - DNA, 21 bp, artificial sequence
  Small Activating RNA
gugauauuguggggcuuuutt

SEQ ID NO: 254 - DNA, 21 bp, artificial sequence
  Small Activating RNA
aaaagccccacaauaucactt

SEQ ID NO: 255 - DNA, 21 bp, artificial sequence
  Small Activating RNA
ggugauauuguggggcuuutt

SEQ ID NO: 256 - DNA, 21 bp, artificial sequence
  Small Activating RNA
aaagccccacaauaucacctt

SEQ ID NO: 257 - DNA, 21 bp, artificial sequence
  Small Activating RNA
aggugauauuguggggcuutt

SEQ ID NO: 258 - DNA, 21 bp, artificial sequence
  Small Activating RNA
aagccccacaauaucaccutt

SEQ ID NO: 259 - DNA, 21 bp, artificial sequence
  Small Activating RNA
gggaauagaggugauauugtt

SEQ ID NO: 260 - DNA, 21 bp, artificial sequence
  Small Activating RNA
caauaucaccucuauuccctt

SEQ ID NO: 261 - DNA, 21 bp, artificial sequence
  Small Activating RNA
ugggaauagaggugauauutt

SEQ ID NO: 262 - DNA, 21 bp, artificial sequence
  Small Activating RNA
aauaucaccucuauucccatt

SEQ ID NO: 263 - DNA, 21 bp, artificial sequence
  Small Activating RNA
gugggaauagaggugauautt

SEQ ID NO: 264 - DNA, 21 bp, artificial sequence
  Small Activating RNA
auaucaccucuauucccactt

SEQ ID NO: 265 - DNA, 21 bp, artificial sequence
  Small Activating RNA
agugggaauagaggugauatt

SEQ ID NO: 266 - DNA, 21 bp, artificial sequence
  Small Activating RNA
uaucaccucuauucccacutt

SEQ ID NO: 267 - DNA, 21 bp, artificial sequence
  Small Activating RNA
cagugggaauagaggugautt

SEQ ID NO: 268 - DNA, 21 bp, artificial sequence
  Small Activating RNA
aucaccucuauucccacugtt

SEQ ID NO: 269 - DNA, 21 bp, artificial sequence
  Small Activating RNA
ucagugggaauagaggugatt

SEQ ID NO: 270 - DNA, 21 bp, artificial sequence
  Small Activating RNA
ucaccucuauucccacugatt

SEQ ID NO: 271 - DNA, 21 bp, artificial sequence
  Small Activating RNA
aucagugggaauagaggugtt

SEQ ID NO: 272 - DNA, 21 bp, artificial sequence
  Small Activating RNA
caccucuauucccacugautt

SEQ ID NO: 273 - DNA, 21 bp, artificial sequence
  Small Activating RNA
gaucagugggaauagaggutt

SEQ ID NO: 274 - DNA, 21 bp, artificial sequence
  Small Activating RNA
accucuauucccacugauctt

SEQ ID NO: 275 - DNA, 21 bp, artificial sequence
  Small Activating RNA
ggaucagugggaauagaggtt

SEQ ID NO: 276 - DNA, 21 bp, artificial sequence
  Small Activating RNA
ccucuauucccacugaucctt

SEQ ID NO: 277 - DNA, 21 bp, artificial sequence
  Small Activating RNA
gggaucagugggaauagagtt

SEQ ID NO: 278 - DNA, 21 bp, artificial sequence
  Small Activating RNA
cucuauucccacugauccctt

SEQ ID NO: 279 - DNA, 21 bp, artificial sequence
  Small Activating RNA
agggaucagugggaauagatt

SEQ ID NO: 280 - DNA, 21 bp, artificial sequence
  Small Activating RNA
ucuauucccacugaucccutt

SEQ ID NO: 281 - DNA, 21 bp, artificial sequence
  Small Activating RNA
gagggaucagugggaauagtt

SEQ ID NO: 282 - DNA, 21 bp, artificial sequence
  Small Activating RNA
cuauucccacugaucccuctt

SEQ ID NO: 283 - DNA, 21 bp, artificial sequence
  Small Activating RNA
ugagggaucagugggaauatt

SEQ ID NO: 284 - DNA, 21 bp, artificial sequence
  Small Activating RNA
uauucccacugaucccucatt

SEQ ID NO: 285 - DNA, 21 bp, artificial sequence
  Small Activating RNA
gugagggaucagugggaautt

SEQ ID NO: 286 - DNA, 21 bp, artificial sequence
  Small Activating RNA
auucccacugaucccucactt

SEQ ID NO: 287 - DNA, 21 bp, artificial sequence
  Small Activating RNA
agugagggaucagugggaatt

SEQ ID NO: 288 - DNA, 21 bp, artificial sequence
  Small Activating RNA
uucccacugaucccucacutt

SEQ ID NO: 289 - DNA, 21 bp, artificial sequence
  Small Activating RNA
uagugagggaucagugggatt

SEQ ID NO: 290 - DNA, 21 bp, artificial sequence
  Small Activating RNA
ucccacugaucccucacuatt

SEQ ID NO: 291 - DNA, 21 bp, artificial sequence
  Small Activating RNA
cuagugagggaucagugggtt

SEQ ID NO: 292 - DNA, 21 bp, artificial sequence
  Small Activating RNA
cccacugaucccucacuagtt

SEQ ID NO: 293 - DNA, 21 bp, artificial sequence
  Small Activating RNA
accuagugagggaucagugtt

SEQ ID NO: 294 - DNA, 21 bp, artificial sequence
  Small Activating RNA
cacugaucccucacuaggutt

SEQ ID NO: 295 - DNA, 21 bp, artificial sequence
  Small Activating RNA
gaccuagugagggaucagutt

SEQ ID NO: 296 - DNA, 21 bp, artificial sequence
  Small Activating RNA
acugaucccucacuagguctt

SEQ ID NO: 297 - DNA, 21 bp, artificial sequence
  Small Activating RNA
ugaccuagugagggaucagtt

SEQ ID NO: 298 - DNA, 21 bp, artificial sequence
  Small Activating RNA
cugaucccucacuaggucatt

SEQ ID NO: 299 - DNA, 21 bp, artificial sequence
  Small Activating RNA
gugaccuagugagggaucatt

SEQ ID NO: 300 - DNA, 21 bp, artificial sequence
  Small Activating RNA
ugaucccucacuaggucactt

SEQ ID NO: 301 - DNA, 21 bp, artificial sequence
  Small Activating RNA
ggugaccuagugagggauctt

SEQ ID NO: 302 - DNA, 21 bp, artificial sequence
  Small Activating RNA
gaucccucacuaggucacctt

SEQ ID NO: 303 - DNA, 21 bp, artificial sequence
  Small Activating RNA
aggugaccuagugagggautt

SEQ ID NO: 304 - DNA, 21 bp, artificial sequence
  Small Activating RNA
aucccucacuaggucaccutt

SEQ ID NO: 305 - DNA, 21 bp, artificial sequence
  Small Activating RNA
gaggugaccuagugagggatt

SEQ ID NO: 306 - DNA, 21 bp, artificial sequence
  Small Activating RNA
ucccucacuaggucaccuctt

SEQ ID NO: 307 - DNA, 21 bp, artificial sequence
  Small Activating RNA
ggagaggugaccuagugagtt

SEQ ID NO: 308 - DNA, 21 bp, artificial sequence
  Small Activating RNA
cucacuaggucaccucucctt

SEQ ID NO: 309 - DNA, 21 bp, artificial sequence
  Small Activating RNA
gggagaggugaccuagugatt

SEQ ID NO: 310 - DNA, 21 bp, artificial sequence
  Small Activating RNA
ucacuaggucaccucuccctt

SEQ ID NO: 311 - DNA, 21 bp, artificial sequence
  Small Activating RNA
ugggagaggugaccuagugtt

SEQ ID NO: 312 - DNA, 21 bp, artificial sequence
  Small Activating RNA
cacuaggucaccucucccatt

SEQ ID NO: 313 - DNA, 21 bp, artificial sequence
  Small Activating RNA
cugggagaggugaccuagutt

SEQ ID NO: 314 - DNA, 21 bp, artificial sequence
  Small Activating RNA
acuaggucaccucucccagtt

SEQ ID NO: 315 - DNA, 21 bp, artificial sequence
  Small Activating RNA
ucugggagaggugaccuagtt

SEQ ID NO: 316 - DNA, 21 bp, artificial sequence
  Small Activating RNA
cuaggucaccucucccagatt

SEQ ID NO: 317 - DNA, 21 bp, artificial sequence
  Small Activating RNA
uucugggagaggugaccuatt

SEQ ID NO: 318 - DNA, 21 bp, artificial sequence
  Small Activating RNA
uaggucaccucucccagaatt

SEQ ID NO: 319 - DNA, 21 bp, artificial sequence
  Small Activating RNA
aggugcuccaggugcuucutt

SEQ ID NO: 320 - DNA, 21 bp, artificial sequence
  Small Activating RNA
agaagcaccuggagcaccutt

SEQ ID NO: 321 - DNA, 21 bp, artificial sequence
  Small Activating RNA
uaggugcuccaggugcuuctt

SEQ ID NO: 322 - DNA, 21 bp, artificial sequence
  Small Activating RNA
gaagcaccuggagcaccuatt

SEQ ID NO: 323 - DNA, 21 bp, artificial sequence
  Small Activating RNA
ugucuaggugcuccaggugtt

SEQ ID NO: 324 - DNA, 21 bp, artificial sequence
  Small Activating RNA
caccuggagcaccuagacatt

SEQ ID NO: 325 - DNA, 21 bp, artificial sequence
  Small Activating RNA
ggugucuaggugcuccaggtt

SEQ ID NO: 326 - DNA, 21 bp, artificial sequence
  Small Activating RNA
ccuggagcaccuagacacctt

SEQ ID NO: 327 - DNA, 21 bp, artificial sequence
  Small Activating RNA
gggugucuaggugcuccagtt

SEQ ID NO: 328 - DNA, 21 bp, artificial sequence
  Small Activating RNA
cuggagcaccuagacaccctt

SEQ ID NO: 329 - DNA, 21 bp, artificial sequence
  Small Activating RNA
ggggugucuaggugcuccatt

SEQ ID NO: 330 - DNA, 21 bp, artificial sequence
  Small Activating RNA
uggagcaccuagacacccctt

SEQ ID NO: 331 - DNA, 21 bp, artificial sequence
  Small Activating RNA
uggggugucuaggugcucctt

SEQ ID NO: 332 - DNA, 21 bp, artificial sequence
  Small Activating RNA
ggagcaccuagacaccccatt

SEQ ID NO: 333 - DNA, 21 bp, artificial sequence
  Small Activating RNA
uuggggugucuaggugcuctt

SEQ ID NO: 334 - DNA, 21 bp, artificial sequence
  Small Activating RNA
gagcaccuagacaccccaatt

SEQ ID NO: 335 - DNA, 21 bp, artificial sequence
  Small Activating RNA
uuguuggggugucuaggugtt

SEQ ID NO: 336 - DNA, 21 bp, artificial sequence
  Small Activating RNA
caccuagacaccccaacaatt

SEQ ID NO: 337 - DNA, 21 bp, artificial sequence
  Small Activating RNA
uuuguuggggugucuaggutt

SEQ ID NO: 338 - DNA, 21 bp, artificial sequence
  Small Activating RNA
accuagacaccccaacaaatt

SEQ ID NO: 339 - DNA, 21 bp, artificial sequence
  Small Activating RNA
cuuuguuggggugucuaggtt

SEQ ID NO: 340 - DNA, 21 bp, artificial sequence
  Small Activating RNA
ccuagacaccccaacaaagtt

SEQ ID NO: 341 - DNA, 21 bp, artificial sequence
  Small Activating RNA
aguggaccucaauuuccuctt

SEQ ID NO: 342 - DNA, 21 bp, artificial sequence
  Small Activating RNA
gaggaaauugagguccacutt

SEQ ID NO: 343 - DNA, 21 bp, artificial sequence
  Small Activating RNA
caguggaccucaauuuccutt

SEQ ID NO: 344 - DNA, 21 bp, artificial sequence
  Small Activating RNA
aggaaauugagguccacugtt

SEQ ID NO: 345 - DNA, 21 bp, artificial sequence
  Small Activating RNA
ucaguggaccucaauuucctt

SEQ ID NO: 346 - DNA, 21 bp, artificial sequence
  Small Activating RNA
ggaaauugagguccacugatt

SEQ ID NO: 347 - DNA, 21 bp, artificial sequence
  Small Activating RNA
uucaguggaccucaauuuctt

SEQ ID NO: 348 - DNA, 21 bp, artificial sequence
  Small Activating RNA
gaaauugagguccacugaatt

SEQ ID NO: 349 - DNA, 21 bp, artificial sequence
  Small Activating RNA
guucaguggaccucaauuutt

SEQ ID NO: 350 - DNA, 21 bp, artificial sequence
  Small Activating RNA
aaauugagguccacugaactt

SEQ ID NO: 351 - DNA, 21 bp, artificial sequence
  Small Activating RNA
aguucaguggaccucaauutt

SEQ ID NO: 352 - DNA, 21 bp, artificial sequence
  Small Activating RNA
aauugagguccacugaacutt

SEQ ID NO: 353 - DNA, 21 bp, artificial sequence
  Small Activating RNA
aaguucaguggaccucaautt

SEQ ID NO: 354 - DNA, 21 bp, artificial sequence
  Small Activating RNA
auugagguccacugaacuutt

SEQ ID NO: 355 - DNA, 21 bp, artificial sequence
  Small Activating RNA
uaaguucaguggaccucaatt

SEQ ID NO: 356 - DNA, 21 bp, artificial sequence
  Small Activating RNA
uugagguccacugaacuuatt

SEQ ID NO: 357 - DNA, 21 bp, artificial sequence
  Small Activating RNA
uuaaguucaguggaccucatt

SEQ ID NO: 358 - DNA, 21 bp, artificial sequence
  Small Activating RNA
ugagguccacugaacuuaatt

SEQ ID NO: 359 - DNA, 21 bp, artificial sequence
  Small Activating RNA
cuuaaguucaguggaccuctt

SEQ ID NO: 360 - DNA, 21 bp, artificial sequence
  Small Activating RNA
gagguccacugaacuuaagtt

SEQ ID NO: 361 - DNA, 21 bp, artificial sequence
  Small Activating RNA
acuuaaguucaguggaccutt

SEQ ID NO: 362 - DNA, 21 bp, artificial sequence
  Small Activating RNA
agguccacugaacuuaagutt

SEQ ID NO: 363 - DNA, 21 bp, artificial sequence
  Small Activating RNA
uacuuaaguucaguggacctt

SEQ ID NO: 364 - DNA, 21 bp, artificial sequence
  Small Activating RNA
gguccacugaacuuaaguatt

SEQ ID NO: 365 - DNA, 21 bp, artificial sequence
  Small Activating RNA
auacuuaaguucaguggactt

SEQ ID NO: 366 - DNA, 21 bp, artificial sequence
  Small Activating RNA
guccacugaacuuaaguautt

SEQ ID NO: 367 - DNA, 21 bp, artificial sequence
  Small Activating RNA
gugacaaucaacaacuuugtt

SEQ ID NO: 368 - DNA, 21 bp, artificial sequence
  Small Activating RNA
caaaguuguugauugucactt

SEQ ID NO: 369 - DNA, 21 bp, artificial sequence
  Small Activating RNA
caugugacaaucaacaacutt

SEQ ID NO: 370 - DNA, 21 bp, artificial sequence
  Small Activating RNA
aguuguugauugucacaugtt

SEQ ID NO: 371 - DNA, 21 bp, artificial sequence
  Small Activating RNA
gcaugugacaaucaacaactt

SEQ ID NO: 372 - DNA, 21 bp, artificial sequence
  Small Activating RNA
guuguugauugucacaugctt

SEQ ID NO: 373 - DNA, 21 bp, artificial sequence
  Small Activating RNA
agcaugugacaaucaacaatt

SEQ ID NO: 374 - DNA, 21 bp, artificial sequence
  Small Activating RNA
uuguugauugucacaugcutt

SEQ ID NO: 375 - DNA, 21 bp, artificial sequence
  Small Activating RNA
aagcaugugacaaucaacatt

SEQ ID NO: 376 - DNA, 21 bp, artificial sequence
  Small Activating RNA
uguugauugucacaugcuutt

SEQ ID NO: 377 - DNA, 21 bp, artificial sequence
  Small Activating RNA
gaagcaugugacaaucaactt

SEQ ID NO: 378 - DNA, 21 bp, artificial sequence
  Small Activating RNA
guugauugucacaugcuuctt

SEQ ID NO: 379 - DNA, 21 bp, artificial sequence
  Small Activating RNA
ggaagcaugugacaaucaatt

SEQ ID NO: 380 - DNA, 21 bp, artificial sequence
  Small Activating RNA
uugauugucacaugcuucctt

SEQ ID NO: 381 - DNA, 21 bp, artificial sequence
  Small Activating RNA
cggaagcaugugacaaucatt

SEQ ID NO: 382 - DNA, 21 bp, artificial sequence
  Small Activating RNA
ugauugucacaugcuuccgtt

SEQ ID NO: 383 - DNA, 21 bp, artificial sequence
  Small Activating RNA
ccggaagcaugugacaauctt

SEQ ID NO: 384 - DNA, 21 bp, artificial sequence
  Small Activating RNA
gauugucacaugcuuccggtt

SEQ ID NO: 385 - DNA, 21 bp, artificial sequence
  Small Activating RNA
cccggaagcaugugacaautt

SEQ ID NO: 386 - DNA, 21 bp, artificial sequence
  Small Activating RNA
auugucacaugcuuccgggtt

SEQ ID NO: 387 - DNA, 21 bp, artificial sequence
  Small Activating RNA
ucccggaagcaugugacaatt

SEQ ID NO: 388 - DNA, 21 bp, artificial sequence
  Small Activating RNA
uugucacaugcuuccgggatt

SEQ ID NO: 389 - DNA, 21 bp, artificial sequence
  Small Activating RNA
uucccggaagcaugugacatt

SEQ ID NO: 390 - DNA, 21 bp, artificial sequence
  Small Activating RNA
ugucacaugcuuccgggaatt

SEQ ID NO: 391 - DNA, 21 bp, artificial sequence
  Small Activating RNA
cuucccggaagcaugugactt

SEQ ID NO: 392 - DNA, 21 bp, artificial sequence
  Small Activating RNA
gucacaugcuuccgggaagtt

SEQ ID NO: 393 - DNA, 21 bp, artificial sequence
  Small Activating RNA
ccuucccggaagcaugugatt

SEQ ID NO: 394 - DNA, 21 bp, artificial sequence
  Small Activating RNA
ucacaugcuuccgggaaggtt

SEQ ID NO: 395 - DNA, 21 bp, artificial sequence
  Small Activating RNA
uccuucccggaagcaugugtt

SEQ ID NO: 396 - DNA, 21 bp, artificial sequence
  Small Activating RNA
cacaugcuuccgggaaggatt

SEQ ID NO: 397 - DNA, 21 bp, artificial sequence
  Small Activating RNA
cuccuucccggaagcaugutt

SEQ ID NO: 398 - DNA, 21 bp, artificial sequence
  Small Activating RNA
acaugcuuccgggaaggagtt

SEQ ID NO: 399 - DNA, 21 bp, artificial sequence
  Small Activating RNA
ccuccuucccggaagcaugtt

SEQ ID NO: 400 - DNA, 21 bp, artificial sequence
  Small Activating RNA
caugcuuccgggaaggaggtt

SEQ ID NO: 401 - DNA, 21 bp, artificial sequence
  Small Activating RNA
cccuccuucccggaagcautt

SEQ ID NO: 402 - DNA, 21 bp, artificial sequence
  Small Activating RNA
augcuuccgggaaggagggtt

SEQ ID NO: 403 - DNA, 21 bp, artificial sequence
  Small Activating RNA
ucccuccuucccggaagcatt

SEQ ID NO: 404 - DNA, 21 bp, artificial sequence
  Small Activating RNA
ugcuuccgggaaggagggatt

SEQ ID NO: 405 - DNA, 21 bp, artificial sequence
  Small Activating RNA
auucccuccuucccggaagtt

SEQ ID NO: 406 - DNA, 21 bp, artificial sequence
  Small Activating RNA
cuuccgggaaggagggaautt

SEQ ID NO: 407 - DNA, 21 bp, artificial sequence
  Small Activating RNA
aauucccuccuucccggaatt

SEQ ID NO: 408 - DNA, 21 bp, artificial sequence
  Small Activating RNA
uuccgggaaggagggaauutt

SEQ ID NO: 409 - DNA, 21 bp, artificial sequence
  Small Activating RNA
ucuccaauucccuccuucctt

SEQ ID NO: 410 - DNA, 21 bp, artificial sequence
  Small Activating RNA
ggaaggagggaauuggagatt

SEQ ID NO: 411 - DNA, 21 bp, artificial sequence
  Small Activating RNA
cucuccaauucccuccuuctt

SEQ ID NO: 412 - DNA, 21 bp, artificial sequence
  Small Activating RNA
gaaggagggaauuggagagtt

SEQ ID NO: 413 - DNA, 21 bp, artificial sequence
  Small Activating RNA
ucucuccaauucccuccuutt

SEQ ID NO: 414 - DNA, 21 bp, artificial sequence
  Small Activating RNA
aaggagggaauuggagagatt

SEQ ID NO: 415 - DNA, 21 bp, artificial sequence
  Small Activating RNA
gucucuccaauucccuccutt

SEQ ID NO: 416 - DNA, 21 bp, artificial sequence
  Small Activating RNA
aggagggaauuggagagactt

SEQ ID NO: 417 - DNA, 21 bp, artificial sequence
  Small Activating RNA
agucucuccaauucccucctt

SEQ ID NO: 418 - DNA, 21 bp, artificial sequence
  Small Activating RNA
ggagggaauuggagagacutt

SEQ ID NO: 419 - DNA, 21 bp, artificial sequence
  Small Activating RNA
uagucucuccaauucccuctt

SEQ ID NO: 420 - DNA, 21 bp, artificial sequence
  Small Activating RNA
gagggaauuggagagacuatt

SEQ ID NO: 421 - DNA, 21 bp, artificial sequence
  Small Activating RNA
guagucucuccaauucccutt

SEQ ID NO: 422 - DNA, 21 bp, artificial sequence
  Small Activating RNA
agggaauuggagagacuactt

SEQ ID NO: 423 - DNA, 21 bp, artificial sequence
  Small Activating RNA
gguagucucuccaauuccctt

SEQ ID NO: 424 - DNA, 21 bp, artificial sequence
  Small Activating RNA
gggaauuggagagacuacctt

SEQ ID NO: 425 - DNA, 21 bp, artificial sequence
  Small Activating RNA
ugcaaccacagggauuucutt

SEQ ID NO: 426 - DNA, 21 bp, artificial sequence
  Small Activating RNA
agaaaucccugugguugcatt

SEQ ID NO: 427 - DNA, 21 bp, artificial sequence
  Small Activating RNA
cugcaaccacagggauuuctt

SEQ ID NO: 428 - DNA, 21 bp, artificial sequence
  Small Activating RNA
gaaaucccugugguugcagtt

SEQ ID NO: 429 - DNA, 21 bp, artificial sequence
  Small Activating RNA
gcugcaaccacagggauuutt

SEQ ID NO: 430 - DNA, 21 bp, artificial sequence
  Small Activating RNA
aaaucccugugguugcagctt

SEQ ID NO: 431 - DNA, 21 bp, artificial sequence
  Small Activating RNA
ugcugcaaccacagggauutt

SEQ ID NO: 432 - DNA, 21 bp, artificial sequence
  Small Activating RNA
aaucccugugguugcagcatt

SEQ ID NO: 433 - DNA, 21 bp, artificial sequence
  Small Activating RNA
cugcugcaaccacagggautt

SEQ ID NO: 434 - DNA, 21 bp, artificial sequence
  Small Activating RNA
aucccugugguugcagcagtt

SEQ ID NO: 435 - DNA, 21 bp, artificial sequence
  Small Activating RNA
gcugcugcaaccacagggatt

SEQ ID NO: 436 - DNA, 21 bp, artificial sequence
  Small Activating RNA
ucccugugguugcagcagctt

SEQ ID NO: 437 - DNA, 21 bp, artificial sequence
  Small Activating RNA
agcugcugcaaccacagggtt

SEQ ID NO: 438 - DNA, 21 bp, artificial sequence
  Small Activating RNA
cccugugguugcagcagcutt

SEQ ID NO: 439 - DNA, 21 bp, artificial sequence
  Small Activating RNA
aaagcugcugcaaccacagtt

SEQ ID NO: 440 - DNA, 21 bp, artificial sequence
  Small Activating RNA
cugugguugcagcagcuuutt

SEQ ID NO: 441 - DNA, 21 bp, artificial sequence
  Small Activating RNA
caaagcugcugcaaccacatt

SEQ ID NO: 442 - DNA, 21 bp, artificial sequence
  Small Activating RNA
ugugguugcagcagcuuugtt

SEQ ID NO: 443 - DNA, 21 bp, artificial sequence
  Small Activating RNA
acaaagcugcugcaaccactt

SEQ ID NO: 444 - DNA, 21 bp, artificial sequence
  Small Activating RNA
gugguugcagcagcuuugutt

SEQ ID NO: 445 - DNA, 21 bp, artificial sequence
  Small Activating RNA
aacaaagcugcugcaaccatt

SEQ ID NO: 446 - DNA, 21 bp, artificial sequence
  Small Activating RNA
ugguugcagcagcuuuguutt

SEQ ID NO: 447 - DNA, 21 bp, artificial sequence
  Small Activating RNA
caacaaagcugcugcaacctt

SEQ ID NO: 448 - DNA, 21 bp, artificial sequence
  Small Activating RNA
gguugcagcagcuuuguugtt

SEQ ID NO: 449 - DNA, 21 bp, artificial sequence
  Small Activating RNA
ccaacaaagcugcugcaactt

SEQ ID NO: 450 - DNA, 21 bp, artificial sequence
  Small Activating RNA
guugcagcagcuuuguuggtt

SEQ ID NO: 451 - DNA, 21 bp, artificial sequence
  Small Activating RNA
gccaacaaagcugcugcaatt

SEQ ID NO: 452 - DNA, 21 bp, artificial sequence
  Small Activating RNA
uugcagcagcuuuguuggctt

SEQ ID NO: 453 - DNA, 21 bp, artificial sequence
  Small Activating RNA
ggccaacaaagcugcugcatt

SEQ ID NO: 454 - DNA, 21 bp, artificial sequence
  Small Activating RNA
ugcagcagcuuuguuggcctt

SEQ ID NO: 455 - DNA, 21 bp, artificial sequence
  Small Activating RNA
uggccaacaaagcugcugctt

SEQ ID NO: 456 - DNA, 21 bp, artificial sequence
  Small Activating RNA
gcagcagcuuuguuggccatt

SEQ ID NO: 457 - DNA, 21 bp, artificial sequence
  Small Activating RNA
cuggccaacaaagcugcugtt

SEQ ID NO: 458 - DNA, 21 bp, artificial sequence
  Small Activating RNA
cagcagcuuuguuggccagtt

SEQ ID NO: 459 - DNA, 21 bp, artificial sequence
  Small Activating RNA
ccuggccaacaaagcugcutt

SEQ ID NO: 460 - DNA, 21 bp, artificial sequence
  Small Activating RNA
agcagcuuuguuggccaggtt

SEQ ID NO: 461 - DNA, 21 bp, artificial sequence
  Small Activating RNA
uuccuggccaacaaagcugtt

SEQ ID NO: 462 - DNA, 21 bp, artificial sequence
  Small Activating RNA
cagcuuuguuggccaggaatt

SEQ ID NO: 463 - DNA, 21 bp, artificial sequence
  Small Activating RNA
cuuccuggccaacaaagcutt

SEQ ID NO: 464 - DNA, 21 bp, artificial sequence
  Small Activating RNA
agcuuuguuggccaggaagtt

SEQ ID NO: 465 - DNA, 21 bp, artificial sequence
  Small Activating RNA
cccuuccuggccaacaaagtt

SEQ ID NO: 466 - DNA, 21 bp, artificial sequence
  Small Activating RNA
cuuuguuggccaggaagggtt

SEQ ID NO: 467 - DNA, 21 bp, artificial sequence
  Small Activating RNA
ccccuuccuggccaacaaatt

SEQ ID NO: 468 - DNA, 21 bp, artificial sequence
  Small Activating RNA
uuuguuggccaggaaggggtt

SEQ ID NO: 469 - DNA, 21 bp, artificial sequence
  Small Activating RNA
uccccuuccuggccaacaatt

SEQ ID NO: 470 - DNA, 21 bp, artificial sequence
  Small Activating RNA
uuguuggccaggaaggggatt

SEQ ID NO: 471 - DNA, 21 bp, artificial sequence
  Small Activating RNA
cuccccuuccuggccaacatt

SEQ ID NO: 472 - DNA, 21 bp, artificial sequence
  Small Activating RNA
uguuggccaggaaggggagtt

SEQ ID NO: 473 - DNA, 21 bp, artificial sequence
  Small Activating RNA
uccuccccuuccuggccaatt

SEQ ID NO: 474 - DNA, 21 bp, artificial sequence
  Small Activating RNA
uuggccaggaaggggaggatt

SEQ ID NO: 475 - DNA, 21 bp, artificial sequence
  Small Activating RNA
auccuccccuuccuggccatt

SEQ ID NO: 476 - DNA, 21 bp, artificial sequence
  Small Activating RNA
uggccaggaaggggaggautt

SEQ ID NO: 477 - DNA, 21 bp, artificial sequence
  Small Activating RNA
ucaaauccuccccuuccugtt

SEQ ID NO: 478 - DNA, 21 bp, artificial sequence
  Small Activating RNA
caggaaggggaggauuugatt

SEQ ID NO: 479 - DNA, 21 bp, artificial sequence
  Small Activating RNA
gucaaauccuccccuuccutt

SEQ ID NO: 480 - DNA, 21 bp, artificial sequence
  Small Activating RNA
aggaaggggaggauuugactt

SEQ ID NO: 481 - DNA, 21 bp, artificial sequence
  Small Activating RNA
cgucaaauccuccccuucctt

SEQ ID NO: 482 - DNA, 21 bp, artificial sequence
  Small Activating RNA
ggaaggggaggauuugacgtt

SEQ ID NO: 483 - DNA, 21 bp, artificial sequence
  Small Activating RNA
ucgucaaauccuccccuuctt

SEQ ID NO: 484 - DNA, 21 bp, artificial sequence
  Small Activating RNA
gaaggggaggauuugacgatt

SEQ ID NO: 485 - DNA, 21 bp, artificial sequence
  Small Activating RNA
cucgucaaauccuccccuutt

SEQ ID NO: 486 - DNA, 21 bp, artificial sequence
  Small Activating RNA
aaggggaggauuugacgagtt

SEQ ID NO: 487 - DNA, 21 bp, artificial sequence
  Small Activating RNA
acucgucaaauccuccccutt

SEQ ID NO: 488 - DNA, 21 bp, artificial sequence
  Small Activating RNA
aggggaggauuugacgagutt

SEQ ID NO: 489 - DNA, 21 bp, artificial sequence
  Small Activating RNA
ucacucgucaaauccuccctt

SEQ ID NO: 490 - DNA, 21 bp, artificial sequence
  Small Activating RNA
gggaggauuugacgagugatt

SEQ ID NO: 491 - DNA, 21 bp, artificial sequence
  Small Activating RNA
cucacucgucaaauccucctt

SEQ ID NO: 492 - DNA, 21 bp, artificial sequence
  Small Activating RNA
ggaggauuugacgagugagtt

SEQ ID NO: 493 - DNA, 21 bp, artificial sequence
  Small Activating RNA
acucacucgucaaauccuctt

SEQ ID NO: 494 - DNA, 21 bp, artificial sequence
  Small Activating RNA
gaggauuugacgagugagutt

SEQ ID NO: 495 - DNA, 21 bp, artificial sequence
  Small Activating RNA
caacucacucgucaaaucctt

SEQ ID NO: 496 - DNA, 21 bp, artificial sequence
  Small Activating RNA
ggauuugacgagugaguugtt

SEQ ID NO: 497 - DNA, 21 bp, artificial sequence
  Small Activating RNA
acaacucacucgucaaauctt

SEQ ID NO: 498 - DNA, 21 bp, artificial sequence
  Small Activating RNA
gauuugacgagugaguugutt

SEQ ID NO: 499 - DNA, 21 bp, artificial sequence
  Small Activating RNA
gacaacucacucgucaaautt

SEQ ID NO: 500 - DNA, 21 bp, artificial sequence
  Small Activating RNA
auuugacgagugaguuguctt

SEQ ID NO: 501 - DNA, 21 bp, artificial sequence
  Small Activating RNA
agacaacucacucgucaaatt

SEQ ID NO: 502 - DNA, 21 bp, artificial sequence
  Small Activating RNA
uuugacgagugaguugucutt

SEQ ID NO: 503 - DNA, 21 bp, artificial sequence
  Small Activating RNA
cagacaacucacucgucaatt

SEQ ID NO: 504 - DNA, 21 bp, artificial sequence
  Small Activating RNA
uugacgagugaguugucugtt

SEQ ID NO: 505 - DNA, 21 bp, artificial sequence
  Small Activating RNA
acagacaacucacucgucatt

SEQ ID NO: 506 - DNA, 21 bp, artificial sequence
  Small Activating RNA
ugacgagugaguugucugutt

SEQ ID NO: 507 - DNA, 21 bp, artificial sequence
  Small Activating RNA
gacagacaacucacucguctt

SEQ ID NO: 508 - DNA, 21 bp, artificial sequence
  Small Activating RNA
gacgagugaguugucuguctt

SEQ ID NO: 509 - DNA, 21 bp, artificial sequence
  Small Activating RNA
agacagacaacucacucgutt

SEQ ID NO: 510 - DNA, 21 bp, artificial sequence
  Small Activating RNA
acgagugaguugucugucutt

SEQ ID NO: 511 - DNA, 21 bp, artificial sequence
  Small Activating RNA
gagacagacaacucacucgtt

SEQ ID NO: 512 - DNA, 21 bp, artificial sequence
  Small Activating RNA
cgagugaguugucugucuctt

SEQ ID NO: 513 - DNA, 21 bp, artificial sequence
  Small Activating RNA
ggagacagacaacucacuctt

SEQ ID NO: 514 - DNA, 21 bp, artificial sequence
  Small Activating RNA
gagugaguugucugucucctt

SEQ ID NO: 515 - DNA, 21 bp, artificial sequence
  Small Activating RNA
aggagacagacaacucacutt

SEQ ID NO: 516 - DNA, 21 bp, artificial sequence
  Small Activating RNA
agugaguugucugucuccutt

SEQ ID NO: 517 - DNA, 21 bp, artificial sequence
  Small Activating RNA
caggagacagacaacucactt

SEQ ID NO: 518 - DNA, 21 bp, artificial sequence
  Small Activating RNA
gugaguugucugucuccugtt

SEQ ID NO: 519 - DNA, 21 bp, artificial sequence
  Small Activating RNA
ucaggagacagacaacucatt

SEQ ID NO: 520 - DNA, 21 bp, artificial sequence
  Small Activating RNA
ugaguugucugucuccugatt

SEQ ID NO: 521 - DNA, 21 bp, artificial sequence
  Small Activating RNA
uucaggagacagacaacuctt

SEQ ID NO: 522 - DNA, 21 bp, artificial sequence
  Small Activating RNA
gaguugucugucuccugaatt

SEQ ID NO: 523 - DNA, 21 bp, artificial sequence
  Small Activating RNA
auucaggagacagacaacutt

SEQ ID NO: 524 - DNA, 21 bp, artificial sequence
  Small Activating RNA
aguugucugucuccugaautt

SEQ ID NO: 525 - DNA, 21 bp, artificial sequence
  Small Activating RNA
uauucaggagacagacaactt

SEQ ID NO: 526 - DNA, 21 bp, artificial sequence
  Small Activating RNA
guugucugucuccugaauatt

SEQ ID NO: 527 - DNA, 21 bp, artificial sequence
  Small Activating RNA
guauucaggagacagacaatt

SEQ ID NO: 528 - DNA, 21 bp, artificial sequence
  Small Activating RNA
uugucugucuccugaauactt

SEQ ID NO: 529 - DNA, 21 bp, artificial sequence
  Small Activating RNA
aguauucaggagacagacatt

SEQ ID NO: 530 - DNA, 21 bp, artificial sequence
  Small Activating RNA
ugucugucuccugaauacutt

SEQ ID NO: 531 - DNA, 21 bp, artificial sequence
  Small Activating RNA
gaguauucaggagacagactt

SEQ ID NO: 532 - DNA, 21 bp, artificial sequence
  Small Activating RNA
gucugucuccugaauacuctt

SEQ ID NO: 533 - DNA, 21 bp, artificial sequence
  Small Activating RNA
ggaguauucaggagacagatt

SEQ ID NO: 534 - DNA, 21 bp, artificial sequence
  Small Activating RNA
ucugucuccugaauacucctt

SEQ ID NO: 535 - DNA, 21 bp, artificial sequence
  Small Activating RNA
gggaguauucaggagacagtt

SEQ ID NO: 536 - DNA, 21 bp, artificial sequence
  Small Activating RNA
cugucuccugaauacuccctt

SEQ ID NO: 537 - DNA, 21 bp, artificial sequence
  Small Activating RNA
ggggaguauucaggagacatt

SEQ ID NO: 538 - DNA, 21 bp, artificial sequence
  Small Activating RNA
ugucuccugaauacucccctt

SEQ ID NO: 539 - DNA, 21 bp, artificial sequence
  Small Activating RNA
uggggaguauucaggagactt

SEQ ID NO: 540 - DNA, 21 bp, artificial sequence
  Small Activating RNA
gucuccugaauacuccccatt

SEQ ID NO: 541 - DNA, 21 bp, artificial sequence
  Small Activating RNA
guggggaguauucaggagatt

SEQ ID NO: 542 - DNA, 21 bp, artificial sequence
  Small Activating RNA
ucuccugaauacuccccactt

SEQ ID NO: 543 - DNA, 21 bp, artificial sequence
  Small Activating RNA
uguggggaguauucaggagtt

SEQ ID NO: 544 - DNA, 21 bp, artificial sequence
  Small Activating RNA
cuccugaauacuccccacatt

SEQ ID NO: 545 - DNA, 21 bp, artificial sequence
  Small Activating RNA
auguggggaguauucaggatt

SEQ ID NO: 546 - DNA, 21 bp, artificial sequence
  Small Activating RNA
uccugaauacuccccacautt

SEQ ID NO: 547 - DNA, 21 bp, artificial sequence
  Small Activating RNA
uauguggggaguauucaggtt

SEQ ID NO: 548 - DNA, 21 bp, artificial sequence
  Small Activating RNA
ccugaauacuccccacauatt

SEQ ID NO: 549 - DNA, 21 bp, artificial sequence
  Small Activating RNA
cuauguggggaguauucagtt

SEQ ID NO: 550 - DNA, 21 bp, artificial sequence
  Small Activating RNA
cugaauacuccccacauagtt

SEQ ID NO: 551 - DNA, 21 bp, artificial sequence
  Small Activating RNA
gcuauguggggaguauucatt

SEQ ID NO: 552 - DNA, 21 bp, artificial sequence
  Small Activating RNA
ugaauacuccccacauagctt

SEQ ID NO: 553 - DNA, 21 bp, artificial sequence
  Small Activating RNA
ggcuauguggggaguauuctt

SEQ ID NO: 554 - DNA, 21 bp, artificial sequence
  Small Activating RNA
gaauacuccccacauagcctt

SEQ ID NO: 555 - DNA, 21 bp, artificial sequence
  Small Activating RNA
gggcuauguggggaguauutt

SEQ ID NO: 556 - DNA, 21 bp, artificial sequence
  Small Activating RNA
aauacuccccacauagccctt

SEQ ID NO: 557 - DNA, 19 bp, artificial sequence
  Small Activating RNA
ccuccugaucuuuucagcc

SEQ ID NO: 558 - DNA, 21 bp, artificial sequence
  Small Activating RNA
agcugaaaagaucaggaggau

SEQ ID NO: 559 - DNA, 19 bp, artificial sequence
  Small Activating RNA
ucugggagaggugaccuaa

SEQ ID NO: 560 - DNA, 21 bp, artificial sequence
  Small Activating RNA
cuaggucaccucucccagaag

SEQ ID NO: 561 - DNA, 19 bp, artificial sequence
  Small Activating RNA
cuaggucaccucucccagc

SEQ ID NO: 562 - DNA, 19 bp, artificial sequence
  Small Activating RNA
cccggaagcaugugacaac

SEQ ID NO: 563 - DNA, 21 bp, artificial sequence
  Small Activating RNA
auugucacaugcuuccgggaa

SEQ ID NO: 564 - DNA, 21 bp, artificial sequence
  Small Activating RNA
gaacccagggagccgacaatt

SEQ ID NO: 565 - DNA, 21 bp, artificial sequence
  Small Activating RNA
uugucggcucccuggguuctt

SEQ ID NO: 566 - DNA, 19 bp, artificial sequence
  Small Activating RNA
gaacccagggagccgacac

SEQ ID NO: 567 - DNA, 21 bp, artificial sequence
  Small Activating RNA
uugucggcucccuggguucuu

SEQ ID NO: 568 - DNA, 21 bp, artificial sequence
  Small Activating RNA
gacgguccugaagagcuaatt

SEQ ID NO: 569 - DNA, 21 bp, artificial sequence
  Small Activating RNA
uuagcucuucaggaccguctt

SEQ ID NO: 570 - DNA, 19 bp, artificial sequence
  Small Activating RNA
gacgguccugaagagcuac

SEQ ID NO: 571 - DNA, 21 bp, artificial sequence
  Small Activating RNA
uuagcucuucaggaccgucag

SEQ ID NO: 572 - DNA, 19 bp, artificial sequence
  Small Activating RNA
gacgguccugaagagcuag

SEQ ID NO: 573 - DNA, 19 bp, artificial sequence
  Small Activating RNA
gacgguccugaagagcuau

SEQ ID NO: 574 - DNA, 21 bp, artificial sequence
  Small Activating RNA
gcaacuccagucccaaauatt

SEQ ID NO: 575 - DNA, 21 bp, artificial sequence
  Small Activating RNA
uauuugggacuggaguugctt

SEQ ID NO: 576 - DNA, 19 bp, artificial sequence
  Small Activating RNA
gcaacuccagucccaaauc

SEQ ID NO: 577 - DNA, 21 bp, artificial sequence
  Small Activating RNA
uauuugggacuggaguugcuu

SEQ ID NO: 578 - DNA, 20 bp, artificial sequence
  Synthesized primer for PCR
ttcccatctcaaggcacacc

SEQ ID NO: 579 - DNA, 20 bp, artificial sequence
  Synthesized primer for PCR
gcgaatttccatccacagcc

SEQ ID NO: 580 - DNA, 20 bp, artificial sequence
  Synthesized primer for PCR
aggcttccccaaacccctaa

SEQ ID NO: 581 - DNA, 20 bp, artificial sequence
  Synthesized primer for PCR
tccgtgagcttgaggttctt

SEQ ID NO: 582 - DNA, 20 bp, artificial sequence
  Synthesized primer for PCR
ctttctgctgtcttgggtgc

SEQ ID NO: 583 - DNA, 21 bp, artificial sequence
  Synthesized primer for PCR
ttcgtgatgattctgccctcc

SEQ ID NO: 584 - RNA, 19 nt, artificial sequence
  Small Activating RNA
cuggagagugccaacucau

SEQ ID NO: 585 - RNA, 19 nt, artificial sequence
  Small Activating RNA
uggagagugccaacucauu

SEQ ID NO: 586 - RNA, 19 nt, artificial sequence
  Small Activating RNA
ggagagugccaacucauuc

SEQ ID NO: 587 - RNA, 19 nt, artificial sequence
  Small Activating RNA
gagagugccaacucauucu

SEQ ID NO: 588 - RNA, 19 nt, artificial sequence
  Small Activating RNA
agagugccaacucauucuc

SEQ ID NO: 589 - RNA, 19 nt, artificial sequence
  Small Activating RNA
gagugccaacucauucucc

SEQ ID NO: 590 - RNA, 19 nt, artificial sequence
  Small Activating RNA
agugccaacucauucucca

SEQ ID NO: 591 - RNA, 19 nt, artificial sequence
  Small Activating RNA
gugccaacucauucuccaa

SEQ ID NO: 592 - RNA, 19 nt, artificial sequence
  Small Activating RNA
ugccaacucauucuccaag

SEQ ID NO: 593 - RNA, 19 nt, artificial sequence
  Small Activating RNA
gccaacucauucuccaagu

SEQ ID NO: 594 - RNA, 19 nt, artificial sequence
  Small Activating RNA
ccaacucauucuccaagua

SEQ ID NO: 595 - RNA, 19 nt, artificial sequence
  Small Activating RNA
caacucauucuccaaguaa

SEQ ID NO: 596 - RNA, 19 nt, artificial sequence
  Small Activating RNA
aaaagccagauuuguggcu

SEQ ID NO: 597 - RNA, 19 nt, artificial sequence
  Small Activating RNA
aaagccagauuuguggcuc

SEQ ID NO: 598 - RNA, 19 nt, artificial sequence
  Small Activating RNA
aagccagauuuguggcuca

SEQ ID NO: 599 - RNA, 19 nt, artificial sequence
  Small Activating RNA
agccagauuuguggcucac

SEQ ID NO: 600 - RNA, 19 nt, artificial sequence
  Small Activating RNA
gccagauuuguggcucacu

SEQ ID NO: 601 - DNA, 1003 bp, Homo sapiens
gcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagct